(19) United States
(12) Patent Application Publication
(10) Pub. No.: US 2025/0009898 A1
(43) Pub. Date: Jan. 9, 2025

(54) PYRROLOBENZODIAZEPINE CONJUGATES FOR CANCER TREATMENT

(71) Applicant: SONY GROUP CORPORATION, Tokyo (JP)

(72) Inventor: Tracy MATRAY, Snohomish, WA (US)

(21) Appl. No.: 18/695,618

(22) PCT Filed: Sep. 27, 2022
(86) PCT No.: PCT/IB2022/059148

§ 371 Date: Mar. 26, 2024

Related U.S. Application Data
(60) Provisional application No. 63/250,931, filed on Sep. 30, 2021.

(51) Int. Cl. A61K 47/68

(52) U.S. Cl. CPC A61K 47/68035; A61K 47/6849; A61K 47/6889

(57) ABSTRACT

Compounds useful as biologically active compounds are disclosed. The compounds have the following structure (I): or a stereoisomer, tautomer, or salt thereof, wherein R¹, R², R³, R⁴, R⁵, L^{1a}, L^{1b}, L², L³, L⁴, L⁵, L⁶, L⁷, M¹, M², q, w, m and n are as defined herein. Compounds of structure (I) find utility in a number of applications, including use as therapeutic agents for various treatment methods.

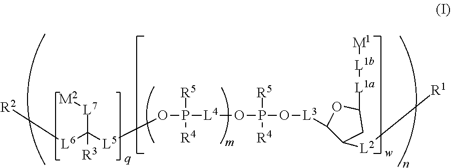

### BACKGROUND

### Field

Embodiments of the present disclosure are generally directed to polymeric biologically active compounds having alkylating agents, and methods for their preparation.

### Description of the Related Art

Targeted drug conjugates, unlike, e.g., chemotherapy, deliver drugs to target cells, with little or no off-target activity. Typically, targeted drug conjugates comprise a targeting molecule that is linked to a biologically active payload or drug. By combining the unique targeting capability with the therapeutic effectiveness of a biologically active drug, conjugates can deliver the drug only to the intended target and minimize potential side effects.

Antibody-drug conjugates (ADCs) are one class of targeted drug conjugates that are of particular interest, for example for cancer treatment. ADCs for cancer treatment combine the targeting features of monoclonal antibodies with cancer-killing ability of cytotoxic agents to provide a therapeutic with several advantages over other chemotherapeutics. However, challenges related to the complexity of ADC constructs, specifically the chemical linker between antibody and drug, has caused significant difficulties for development of new and effective therapeutics. Although the first ADC was approved in 2001, it took almost a decade before the next ADC was approved. As of today, only Adcetris®, Besponsa®, Enhertu®, Mylotarg®, Padcev®, Polivy®, and Kadcyla® are commercially available globally (Zevalin® has been approved in China only).

Thus, there exists a need in the art for developing potent, targeting drug conjugates having a high therapeutic index and methods of preparing the same. The present disclosure fulfills this need and provides further related advantages.

### BRIEF SUMMARY

In one embodiment, compounds having the following structure (I) are provided:

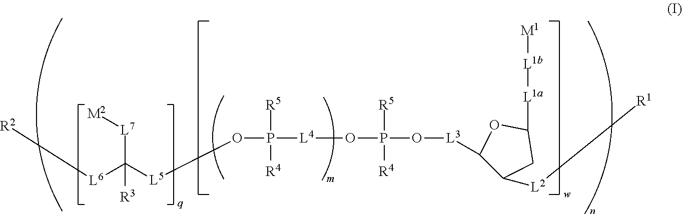

or a stereoisomer, tautomer, or salt thereof, wherein R¹, R², R³, R⁴, R⁵, L^{1a}, L^{1b}, L², L³, L⁴, L⁵, L⁶, L⁷, M¹, M², q, w, m and n are as defined herein. Compounds of structure (I) find utility in a number of applications, including use as therapeutic agents for various treatment methods.

In another embodiment, compositions are provided which comprise a compound of structure (I) and a pharmaceutically acceptable carrier.

In yet another embodiment, a method of treating a disease is provided, the method comprising administering to a subject in need thereof a therapeutically effective amount of a compound of structure (I) or a composition comprising a compound of structure (I), wherein each M1 or M2 is independently a biologically active moiety effective for treating the disease.

In yet another embodiment, compounds having the following Structure (II) are provided.

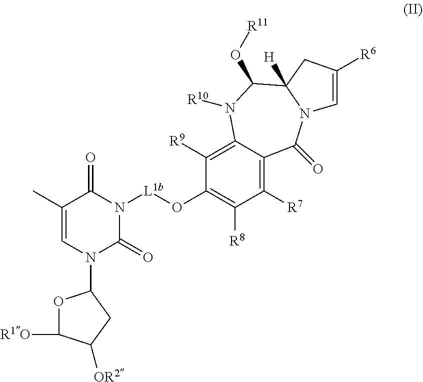

or salt, tautomer, or stereoisomer thereof, wherein R¹″, R²″, L^{1b}, R⁶, R⁷, R⁸, R⁹, R¹⁰, and R¹¹ are as defined herein.

In yet another embodiment, compounds having the following Structure (III) are provided.

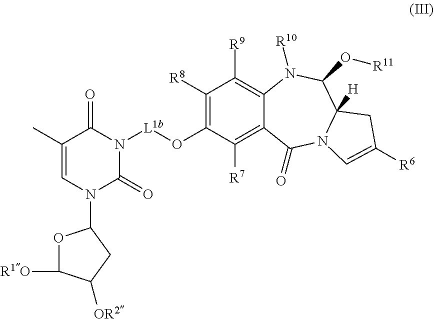

or salt, tautomer, or stereoisomer thereof, wherein R¹″, R²″, L^{1b}, R⁶, R⁷, R⁸, R⁹, R¹⁰, and R¹¹ are as defined herein.

These and other aspects of the disclosure will be apparent upon reference to the following detailed description.

### DETAILED DESCRIPTION

In the following description, certain specific details are set forth in order to provide a thorough understanding of various embodiments of the disclosure. However, one skilled in the art will understand that the disclosure may be practiced without these details.

Unless the context requires otherwise, throughout the present specification and claims, the word “comprise” and variations thereof, such as, “comprises” and “comprising” are to be construed in an open, inclusive sense, that is, as “including, but not limited to”.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

“Amino” refers to the —NH₂ group.

“Carboxy” refers to the —CO₂H group.

“Cyano” refers to the —CN group.

“Formyl” refers to the —C(═O)H group.

“Hydroxy” or “hydroxyl” refers to the —OH group.

“Imino” refers to the =NH group.

“Nitro” refers to the —NO₂ group.

“Oxo” refers to the =O substituent group.

“Sulfhydryl” refers to the —SH group.

“Thioxo” refers to the =S group.

“Alkyl” refers to a straight or branched hydrocarbon chain group consisting solely of carbon and hydrogen atoms, containing no unsaturation, having from one to twelve carbon atoms (C₁-C₁₂ alkyl), one to eight carbon atoms (C₁-C₈ alkyl) or one to six carbon atoms (C₁-C₆ alkyl), and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, alkyl groups are optionally substituted.

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, alkylene is optionally substituted.

“Alkenylene” or “alkenylene chain” refers to a straight or branched divalent hydrocarbon chain linking the rest of the molecule to a radical group, consisting solely of carbon and hydrogen, containing at least one carbon-carbon double bond and having from two to twelve carbon atoms, e.g., ethenylene, propenylene, n-butenylene, and the like. The alkenylene chain is attached to the rest of the molecule through a single bond and to the radical group through a double bond or a single bond. The points of attachment of the alkenylene chain to the rest of the molecule and to the radical group can be through one carbon or any two carbons within the chain. Unless stated otherwise specifically in the specification, alkenylene is optionally substituted.

“Alkynylene” or “alkynylene chain” refers to a straight or branched divalent hydrocarbon chain linking the rest of the molecule to a radical group, consisting solely of carbon and hydrogen, containing at least one carbon-carbon triple bond and having from two to twelve carbon atoms, e.g., ethenylene, propenylene, n-butenylene, and the like. The alkynylene chain is attached to the rest of the molecule through a single bond and to the radical group through a double bond or a single bond. The points of attachment of the alkynylene chain to the rest of the molecule and to the radical group can be through one carbon or any two carbons within the chain. Unless stated otherwise specifically in the specification, alkynylene is optionally substituted.

“Alkylether” refers to any alkyl group as defined above, wherein at least one carbon-carbon bond is replaced with a carbon-oxygen bond. The carbon-oxygen bond may be on the terminal end (as in an alkoxy group) or the carbon oxygen bond may be internal (i.e., C—O—C). Alkylethers include at least one carbon oxygen bond, but may include more than one. For example, polyethylene glycol (PEG) is included within the meaning of alkylether. Unless stated otherwise specifically in the specification, an alkylether group is optionally substituted. For example, in some embodiments an alkylether is substituted with an alcohol or —OP(═Rₐ)(R_{b})R_{c}, wherein each of Rₐ, R_{b} and R_{c} is as defined for compounds of structure (I).

“Alkoxy” refers to a group of the formula —ORₐ where Rₐ is an alkyl group as defined above containing one to twelve carbon atoms. Unless stated otherwise specifically in the specification, an alkoxy group is optionally substituted.

“Alkoxyalkylether” refers to a group of the formula —ORₐR_{b} where Rₐ is an alkylene group as defined above containing one to twelve carbon atoms, and R_{b} is an alkylether group as defined herein. Unless stated otherwise specifically in the specification, an alkoxyalkylether group is optionally substituted, for example substituted with an alcohol or —OP(═Rₐ)(R_{b})R_{c}, wherein each of Rₐ, R_{b} and R_{c} is as defined for compounds of structure (I).

“Heteroalkyl” refers to an alkyl group, as defined above, comprising at least one heteroatom (e.g., N, O, P or S) within the alkyl group or at a terminus of the alkyl group. In some embodiments, the heteroatom is within the alkyl group (i.e., the heteroalkyl comprises at least one carbon-[heteroatom]ₓ-carbon bond, where x is 1, 2 or 3). In other embodiments, the heteroatom is at a terminus of the alkyl group and thus serves to join the alkyl group to the remainder of the molecule (e.g., M1-H-A), where M1 is a portion of the molecule, H is a heteroatom and A is an alkyl group). Unless stated otherwise specifically in the specification, a heteroalkyl group is optionally substituted. Exemplary heteroalkyl groups include ethylene oxide (e.g., polyethylene oxide), optionally including phosphorous-oxygen bonds, such as phosphodiester bonds.

“Heteroalkoxy” refers to a group of the formula —ORₐ where Rₐ is a heteroalkyl group as defined above containing one to twelve carbon atoms. Unless stated otherwise specifically in the specification, a heteroalkoxy group is optionally substituted.

“Heteroalkylene” refers to an alkylene group, as defined above, comprising at least one heteroatom (e.g., Si, N, O, P or S) within the alkylene chain or at a terminus of the alkylene chain. In some embodiments, the heteroatom is within the alkylene chain (i.e., the heteroalkylene comprises at least one carbon-[heteroatom]-carbon bond, where x is 1, 2 or 3). In other embodiments, the heteroatom is at a terminus of the alkylene and thus serves to join the alkylene to the remainder of the molecule (e.g., M1-H-A-M2, where M1 and M2 are portions of the molecule, H is a heteroatom and A is an alkylene). Unless stated otherwise specifically in the specification, a heteroalkylene group is optionally substituted. Exemplary heteroalkylene groups include ethylene oxide (e.g., polyethylene oxide) and the “C,” “HEG,” and “PEG 1K” linking groups illustrated below:

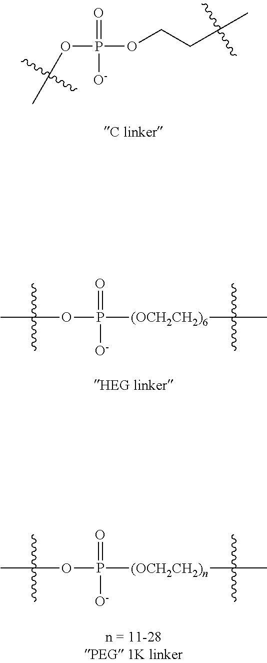

Multimers of the above C-linker, HEG linker and/or PEG 1K linker are included in various embodiments of heteroalkylene linkers. In some embodiments of the PEG 1K linker, n ranges from 19-25, for example n is 19, 20, 21, 22, 23, 24, or 25. Multimers may comprise, for example, the following structure:

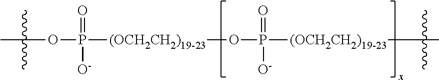

wherein x is 0 or an integer greater than 0, for example, x ranges from 0-100 (e.g., 1, 2, 3, 4, 5, 6, 7, 8, 9 or 10).

A “linker” refers to a contiguous chain of at least one atom, such as carbon, oxygen, nitrogen, sulfur, phosphorous, and combinations thereof, which connects a portion of a molecule to another portion of the same molecule or to a different molecule, moiety or solid support (e.g., microparticle). Linkers may connect the molecule via a covalent bond or other means, such as ionic or hydrogen bond interactions. In some embodiments, the linker is a heteroatomic linker (e.g., comprising 1-10 Si, N, O, P, or S atoms), a heteroalkylene (e.g., comprising 1-10 Si, N, O, P, or S atoms and an alkylene chain) or an alkylene linker (e.g., comprising 1-12 carbon atoms). In some embodiments, a heteroalkylene linker comprises the following structure:

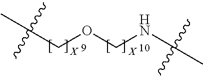

wherein:
x⁹ and x¹⁰ are each independently a integer greater than 0. In some embodiments, the heteroatomic linker is —O—, —S—, or —OP(═O)O—O—. In some embodiments, the heteroalkylene linker comprises —OP(═O)O—O—. In some embodiments, the heteroalkylene linker comprises at least one S—S bond.

“Physiologically cleavable linker” refers to a molecular linkage that can be split or separated a prescribed manner, resulting in two or more separate molecules while in the presence of an in vivo or in vitro environment of an organism or cell system. Generally, physiological conditions that induce such a cleavage or scission event may include a temperature ranging from about 20 to 40° C., an atmospheric pressure of about 1 atm (101 kPa or 14.7 psi), a pH of about 6 to 8, a glucose concentration of about 1 to 20 mM, atmospheric oxygen concentration, and earth gravity. In some embodiments, physiological conditions include enzymatic conditions (i.e., enzymatic cleavage). Bond cleavage or scission can be homolytic or heterolytic.

“Heteroalkenylene” is a heteroalkylene, as defined above, comprising at least one carbon-carbon double bond. Unless stated otherwise specifically in the specification, a heteroalkenylene group is optionally substituted.

“Heteroalkynylene” is a heteroalkylene comprising at least one carbon-carbon triple bond. Unless stated otherwise specifically in the specification, a heteroalkynylene group is optionally substituted.

“Heteroatomic” in reference to a “heteroatomic linker” refers to a linker group consisting of one or more heteroatoms. Exemplary heteroatomic linkers include single atoms selected from the group consisting of O, N, P and S, and multiple heteroatoms for example a linker having the formula —P(O—)(=O)O— or —OP(O—)(=O)O— and multimers and combinations thereof.

“Phosphate” refers to the —OP(═O)(Rₐ)R_{b} group, wherein Rₐ is OH, O— or OR_{c}; and R_{b} is OH, O—, OR_{c}, a thiophosphate group or a further phosphate group, wherein R_{c} is a counter ion (e.g., Na⁺ and the like).

“Phosphoalkyl” refers to the —OP(═O)(Rₐ)R_{b} group, wherein Rₐ is OH, O— or OR_{c}; and R_{b} is —Oalkyl, wherein R_{c} is a counter ion (e.g., Na⁺ and the like). Unless stated otherwise specifically in the specification, a phosphoalkyl group is optionally substituted. For example, in certain embodiments, the —Oalkyl moiety in a phosphoalkyl group is optionally substituted with one or more of hydroxyl, amino, sulfhydryl, phosphate, thiophosphate, phosphoalkyl, thiophosphoalkyl, phosphoalkylether, thiophosphoalkylether or —OP(═Rₐ)(R_{b})R_{c}, wherein each of Rₐ, R_{b} and R_{c} is as defined for compounds of structure (I).

“Phosphoalkylether” refers to the —OP(═O)(Rₐ)R_{b} group, wherein Rₐ is OH, O— or OR_{c}; and R_{b} is —Oalkylether, wherein R_{c} is a counter ion (e.g., Na⁺ and the like). Unless stated otherwise specifically in the specification, a phosphoalkylether group is optionally substituted. For example, in certain embodiments, the —Oalkylether moiety in a phosphoalkylether group is optionally substituted with one or more of hydroxyl, amino, sulfhydryl, phosphate, thiophosphate, phosphoalkyl, thiophosphoalkyl, phosphoalkylether, thiophosphoalkylether or —OP(═Rₐ)(R_{b})R_{c}, wherein each of Rₐ, R_{b} and R_{c} is as defined for compounds of structure (I).

“Thiophosphate” refers to the —OP(═Rₐ)(R_{b})R_{c} group, wherein Rₐ is O or S, R_{b} is OH, O—, S—, OR_{d} or SR_{d}; and R_{c} is OH, SH, O—, S—, OR_{d}, SR_{d}, a phosphate group or a further thiophosphate group, wherein R_{d} is a counter ion (e.g., Na⁺ and the like) and provided that: i) Rₐ is S; ii) R_{b} is S— or SR_{d}; iii) R_{c} is SH, S— or SR_{d}; or iv) a combination of i), ii) and/or iii).

“Thiophosphoalkyl” refers to the —OP(═Rₐ)(R_{b})R_{c} group, wherein Rₐ is O or S, R_{b} is OH, O—, S—, OR_{d} or SR_{d}; and R_{c} is —Oalkyl, wherein R_{d} is a counter ion (e.g., Na⁺ and the like) and provided that: i) Rₐ is S; ii) R_{b} is S— or SR_{d}; or iii) Rₐ is S and R_{b} is S— or SR_{d}. Unless stated otherwise specifically in the specification, a thiophosphoalkyl group is optionally substituted. For example, in certain embodiments, the —Oalkyl moiety in a thiophosphoalkyl group is optionally substituted with one or more of hydroxyl, amino, sulfhydryl, phosphate, thiophosphate, phosphoalkyl, thiophosphoalkyl, phosphoalkylether, thiophosphoalkylether or —OP(═Rₐ)(R_{b})R_{c}, wherein each of Rₐ, R_{b} and R_{c} is as defined for compounds of structure (I).

“Thiophosphoalkylether” refers to the —OP(═Rₐ)(R_{b})R_{c} group, wherein Rₐ is O or S, R_{b} is OH, O—, S—, OR_{d} or SR_{d}; and R_{c} is —Oalkylether, wherein R_{d} is a counter ion (e.g., Na⁺ and the like) and provided that: i) Rₐ is S; ii) R_{b} is S— or SR_{d}; or iii) Rₐ is S and R_{b} is S— or SR_{d}. Unless stated otherwise specifically in the specification, a thiophosphoalkylether group is optionally substituted. For example, in certain embodiments, the —Oalkylether moiety in a thiophosphoalkyl group is optionally substituted with one or more of hydroxyl, amino, sulfhydryl, phosphate, thiophosphate, phosphoalkyl, thiophosphoalkyl, phosphoalkylether, thiophosphoalkylether or —OP(═Rₐ)(R_{b})R_{c}, wherein each of Rₐ, R_{b} and R_{c} is as defined for compounds of structure (I).

“Carbocyclic” refers to a stable 3- to 18-membered aromatic or non-aromatic ring comprising 3 to 18 carbon atoms. Unless stated otherwise specifically in the specification, a carbocyclic ring may be a monocyclic, bicyclic, tricyclic or tetracyclic ring system, which may include fused or bridged ring systems, and may be partially or fully saturated. Non-aromatic carbocyclyl radicals include cycloalkyl, while aromatic carbocyclyl radicals include aryl. Unless stated otherwise specifically in the specification, a carbocyclic group is optionally substituted.

“Cycloalkyl” refers to a stable non-aromatic monocyclic or polycyclic carbocyclic ring, which may include fused or bridged ring systems, having from three to fifteen carbon atoms, preferably having from three to ten carbon atoms, and which is saturated or unsaturated and attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo-[2.2.1]heptanyl, and the like. Unless stated otherwise specifically in the specification, a cycloalkyl group is optionally substituted.

“Aryl” refers to a ring system comprising at least one carbocyclic aromatic ring. In some embodiments, an aryl comprises from 6 to 18 carbon atoms. The aryl ring may be a monocyclic, bicyclic, tricyclic or tetracyclic ring system, which may include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group is optionally substituted.

“Heterocyclic” refers to a stable 3- to 18-membered aromatic or non-aromatic ring comprising one to twelve carbon atoms and from one to six heteroatoms selected from the group consisting of nitrogen, oxygen and sulfur. Unless stated otherwise specifically in the specification, the heterocyclic ring may be a monocyclic, bicyclic, tricyclic or tetracyclic ring system, which may include fused or bridged ring systems; and the nitrogen, carbon or sulfur atoms in the heterocyclic ring may be optionally oxidized; the nitrogen atom may be optionally quaternized; and the heterocyclic ring may be partially or fully saturated. Examples of aromatic heterocyclic rings are listed below in the definition of heteroaryls (i.e., heteroaryl being a subset of heterocyclic). Examples of non-aromatic heterocyclic rings include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, pyrazolopyrimidinyl, quinuclidinyl, thiazolidinyl, tetrahydrofuryl, trioxanyl, trithianyl, triazinanyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclic group is optionally substituted.

“Heteroaryl” refers to a 5- to 14-membered ring system comprising one to thirteen carbon atoms, one to six heteroatoms selected from the group consisting of nitrogen, oxygen and sulfur, and at least one aromatic ring. For purposes of certain embodiments of this disclosure, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, benzoxazolinonyl, benzimidazolthionyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, pteridinonyl, purinyl, pyrrolyl, pyrazolyl, pyridinyl, pyridinonyl, pyrazinyl, pyrimidinyl, pryrimidinonyl, pyridazinyl, pyrrolyl, pyrido[2,3-d]pyrimidinonyl, quinazolinyl, quinazolinonyl, quinoxalinyl, quinoxalinonyl, quinolinyl, isoquinolinyl, tetrahydroquinolinyl, thiazolyl, thiadiazolyl, thieno[3,2-d]pyrimidin-4-onyl, thieno[2,3-d]pyrimidin-4-onyl, triazolyl, tetrazolyl, triazinyl, and thiophenyl (i.e. thienyl). Unless stated otherwise specifically in the specification, a heteroaryl group is optionally substituted.

The suffix “-ene” refers to a particular structural feature (e.g., alkyl, aryl, heteroalkyl, heteroaryl) attached to the rest of the molecule through a single bond and attached to a radical group through a single bond. In other words, the suffix “-ene” refers to a linker having the structural features of the moiety to which it is attached. The points of attachment of the “-ene” chain to the rest of the molecule and to the radical group can be through one atom of or any two atoms within the chain. For example, a heteroarylene refers to a linker comprising a heteroaryl moiety as defined herein.

“Fused” refers to a ring system comprising at least two rings, wherein the two rings share at least one common ring atom, for example two common ring atoms. When the fused ring is a heterocyclyl ring or a heteroaryl ring, the common ring atom(s) may be carbon or nitrogen. Fused rings include bicyclic, tricyclic, tertracyclic, and the like.

The term “substituted” used herein means any of the above groups (e.g., alkyl, alkylene, alkenylene, alkynylene, heteroalkylene, heteroalkenylene, heteroalkynylene, alkoxy, alkylether, phosphoalkyl, phosphoalkylether, thiophosphoalkyl, thiophosphoalkylether, carbocyclic, cycloalkyl, aryl, heterocyclic and/or heteroaryl) wherein at least one hydrogen atom (e.g., 1, 2, 3 or all hydrogen atoms) is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, =NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl group. In addition, each of the foregoing substituents may also be optionally substituted with one or more of the above substituents.

“Conjugation” refers to the overlap of one p-orbital with another p-orbital across an intervening sigma bond. Conjugation may occur in cyclic or acyclic compounds. A “degree of conjugation” refers to the overlap of at least one p-orbital with another p-orbital across an intervening sigma bond. For example, 1, 3-butadine has one degree of conjugation, while benzene and other aromatic compounds typically have multiple degrees of conjugation. Fluorescent and colored compounds typically comprise at least one degree of conjugation.

“Fluorescent” refers to a molecule which is capable of absorbing light of a particular frequency and emitting light of a different frequency. Fluorescence is well-known to those of ordinary skill in the art.

“Colored” refers to a molecule which absorbs light within the colored spectrum (i.e., red, yellow, blue and the like).

The term “biomolecule” refers to any of a variety of biological materials, including nucleic acids, carbohydrates, amino acids, polypeptides, glycoproteins, hormones, aptamers and mixtures thereof. [symbol] More specifically, the term is intended to include, without limitation, RNA, [symbol] DNA, oligonucleotides, modified or derivatized nucleotides, enzymes, receptors, prions, receptor ligands (including hormones), antibodies, antigens, and toxins, as well as bacteria, viruses, blood cells, and tissue cells. The visually detectable biomolecules of the disclosure (e.g., compounds of structure (I) having a biomolecule linked thereto) are prepared, as further described herein, by contacting a biomolecule with a compound having a reactive group that enables attachment of the biomolecule to the compound via any available atom or functional group, such as an amino, hydroxy, carboxyl, or sulfhydryl group on the biomolecule.

A “reactive group” is a moiety capable of reacting with a second reactive groups (e.g., a “complementary reactive group”) to form one or more covalent bonds, for example by a displacement, oxidation, reduction, addition or cycloaddition reaction. Exemplary reactive groups are provided in Table 1, and include for example, nucleophiles, electrophiles, dienes, dienophiles, aldehyde, oxime, hydrazone, alkyne, amine, azide, acylazide, acylhalide, nitrile, nitrone, sulfhydryl, disulfide, sulfonyl halide, isothiocyanate, imidoester, activated ester, ketone, α,β-unsaturated carbonyl, alkene, maleimide, α-haloimide, epoxide, aziridine, tetrazine, tetrazole, phosphine, biotin, thiirane and the like

[symbol]

“Bio-conjugation” or “bio-conjugate” and related variations refer to a chemical reaction strategy for forming a stable covalent bond between two molecules. The term “bio-conjugation” is generally used when one of the molecules is a biomolecule (e.g., an antibody), but can be used to describe forming a covalent bond with a non-biomolecule (e.g., a polymeric resin). The product or compound resulting from such a reaction strategy is a “conjugate,” “bio-conjugate” or a grammatical equivalent.

The terms “visible” and “visually detectable” are used herein to refer to substances that are observable by visual inspection, without prior illumination, or chemical or enzymatic activation. Such visually detectable substances absorb and emit light in a region of the spectrum ranging from about 300 to about 900 nm. Preferably, such substances are intensely colored, preferably having a molar extinction coefficient of at least about 40,000, more preferably at least about 50,000, still more preferably at least about 60,000, yet still more preferably at least about 70,000, and most preferably at least about 80,000 M⁻¹cm⁻¹. The compounds of the disclosure may be detected by observation with the naked eye, or with the aid of an optically based detection device, including, without limitation, absorption spectrophotometers, transmission light microscopes, digital cameras and scanners. Visually detectable substances are not limited to those which emit and/or absorb light in the visible spectrum. Substances which emit and/or absorb light in the ultraviolet (UV) region (about 10 nm to about 400 nm), infrared (IR) region (about 700 nm to about 1 mm), and substances emitting and/or absorbing in other regions of the electromagnetic spectrum are also included with the scope of “visually detectable” substances.

For purposes of embodiments of the disclosure, the term “photostable visible dye” refers to a chemical moiety that is visually detectable, as defined hereinabove, and is not significantly altered or decomposed upon exposure to light. Preferably, the photostable visible dye does not exhibit significant bleaching or decomposition after being exposed to light for at least one hour. More preferably, the visible dye is stable after exposure to light for at least 12 hours, still more preferably at least 24 hours, still yet more preferably at least one week, and most preferably at least one month. Non-limiting examples of photostable visible dyes suitable for use in the compounds and methods of the disclosure include azo dyes, thioindigo dyes, quinacridone pigments, dioxazine, phthalocyanine, perinone, diketopyrrolopyrrole, quinophthalone, and truarycarbonium.

As used herein, the term “perylene derivative” is intended to include any substituted perylene that is visually detectable. However, the term is not intended to include perylene itself. The terms “anthracene derivative”, “naphthalene derivative”, and “pyrene derivative” are used analogously. In some preferred embodiments, a derivative (e.g., perylene, pyrene, anthracene or naphthalene derivative) is an imide, bisimide or hydrazamimide derivative of perylene, anthracene, naphthalene, or pyrene.

[symbol]

The visually detectable molecules of various embodiments of the disclosure are useful for a wide variety of analytical applications, such as biochemical and biomedical applications, in which there is a need to determine the presence, location, or quantity of a particular analyte (e.g., biomolecule). In another aspect, therefore, the disclosure provides a method for visually detecting a biomolecule, comprising: (a) providing a biological system with a visually detectable biomolecule comprising the compound of structure (I) linked to a biomolecule; and (b) detecting the biomolecule by its visible properties. For purposes of the disclosure, the phrase “detecting the biomolecule by its visible properties” means that the biomolecule, without illumination or chemical or enzymatic activation, is observed with the naked eye, or with the aid of a optically based detection device, including, without limitation, absorption spectrophotometers, transmission light microscopes, digital cameras and scanners. A densitometer may be used to quantify the amount of visually detectable biomolecule present. For example, the relative quantity of the biomolecule in two samples can be determined by measuring relative optical density. If the stoichiometry of dye molecules per biomolecule is known, and the extinction coefficient of the dye molecule is known, then the absolute concentration of the biomolecule can also be determined from a measurement of optical density. As used herein, the term “biological system” is used to refer to any solution or mixture comprising one or more biomolecules in addition to the visually detectable biomolecule. Nonlimiting examples of such biological systems include cells, cell extracts, tissue samples, electrophoretic gels, assay mixtures, and hybridization reaction mixtures.

“Solid support” or “solid resin” refers to any solid substrate known in the art for solid-phase support of molecules, for example a “microparticle” refers to any of a number of small particles useful for attachment to compounds of the disclosure, including, but not limited to, glass beads, magnetic beads, polymeric beads, nonpolymeric beads, and the like. In certain embodiments, a microparticle comprises polystyrene beads. In some embodiments, the solid support or solid resin is controlled pore glass or macroporous polystyrene.

A “solid support residue” refers to the functional group remaining attached to a molecule when the molecule is cleaved from the solid support. Solid support residues are known in the art and can be easily derived based on the structure of the solid support and the group linking the molecule thereto.

A “targeting moiety” is a moiety that selectively binds or associates with a particular target, such as an analyte molecule. “Selectively” binding or associating means a targeting moiety preferentially associates or binds with the desired target relative to other targets. In some embodiments the compounds disclosed herein include linkages to targeting moieties for the purpose of selectively binding or associating the compound with an analyte of interest (i.e., the target of the targeting moiety), thus allowing detection of the analyte. Exemplary targeting moieties include, but are not limited to, antibodies, antigens, nucleic acid sequences, enzymes, proteins, cell surface receptor antagonists, and the like. In some embodiments, the targeting moiety is a moiety, such as an antibody, that selectively binds or associates with a target feature on or in a cell, for example a target feature on a cell membrane or other cellular structure, thus allowing for detection of cells of interest. Small molecules that selectively bind or associate with a desired analyte are also contemplated as targeting moieties in certain embodiments. One of skill in the art will understand other analytes, and the corresponding targeting moiety, that will be useful in various embodiments.

“Base pairing moiety” refers to a heterocyclic moiety capable of hybridizing with a complementary heterocyclic moiety via hydrogen bonds (e.g., Watson-Crick base pairing). Base pairing moieties include natural and unnatural bases. Non-limiting examples of base pairing moieties are RNA and DNA bases such adenosine, guanosine, thymidine, cytosine and uridine and analogues thereof.

Embodiments of the disclosure disclosed herein are also meant to encompass all compounds being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²p, ³⁵S, ¹⁸F, ³⁶Cl, ¹²³I and ¹²⁵I respectively.

Isotopically-labeled compounds of structure (I) can generally be prepared by conventional techniques known to those skilled in the art or by processes analogous to those described below and in the following Examples using an appropriate isotopically-labeled reagent in place of the non-labeled reagent previously employed.

“Stable compound” and “stable structure” are meant to indicate a compound that is sufficiently robust to survive isolation to a useful degree of purity from a reaction mixture, and formulation into an efficacious therapeutic agent.

“Optional” or “optionally” means that the subsequently described event or circumstances may or may not occur, and that the description includes instances where said event or circumstance occurs and instances in which it does not. For example, “optionally substituted alkyl” means that the alkyl group may or may not be substituted and that the description includes both substituted alkyl groups and alkyl groups having no substitution.

“Salt” includes both acid and base addition salts.

“Acid addition salt” refers to those salts which are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Base addition salt” refers to those salts which are prepared from addition of an inorganic base or an organic base to the free acid. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Crystallizations may produce a solvate of the compounds described herein. Embodiments of the present disclosure include all solvates of the described compounds. As used herein, the term “solvate” refers to an aggregate that comprises one or more molecules of a compound of the disclosure with one or more molecules of solvent. The solvent may be water, in which case the solvate may be a hydrate. Alternatively, the solvent may be an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. The compounds of the disclosure may be true solvates, while in other cases the compounds of the disclosure may merely retain adventitious water or another solvent or be a mixture of water plus some adventitious solvent.

Embodiments of the compounds of the disclosure (e.g., compounds of structure I), or their salts, tautomers or solvates may contain one or more stereocenters and may thus give rise to enantiomers, diastereomers, and other stereoisomeric forms that may be defined, in terms of absolute stereochemistry, as (R)- or (S)- or, as (D)- or (L)- for amino acids. Embodiments of the present disclosure are meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

A “tautomer” refers to a proton shift from one atom of a molecule to another atom of the same molecule. The present disclosure includes tautomers of any said compounds. Various tautomeric forms of the compounds are easily derivable by those of ordinary skill in the art.

The chemical naming protocol and structure diagrams used herein are a modified form of the I.U.P.A.C. nomenclature system, using the ACD/Name Version 9.07 software program and/or ChemDraw Ultra Version 11.0 software naming program (CambridgeSoft). Common names familiar to one of ordinary skill in the art are also used.

### Compounds

As noted above, in one embodiment of the present disclosure, compounds useful as covalent linkers between biologically active moieties such as alkylating agents and targeting moieties are provided. In other embodiments, compounds useful as synthetic intermediates for preparation of compounds comprising one or more biologically active moieties are provided. Accordingly, in some embodiments, M¹ and M² are, at each occurrence, independently a moiety comprising an alkylating agent or a fluorescent dye, provided that at least one occurrence of M¹ or M² is not a fluorescent dye. In some embodiments, M¹ or M² is an alkylating agent (e.g., pyrrolo benzo diazepine (PBD) and the like).

Numerous advantages are afforded by embodiments disclosed herein, including the ability to control the number of biologically active moieties M¹ and M² that are attached to the polymer backbone and any subsequent targeting moiety, spacing between the neighboring biologically active moieties on the polymer backbone (e.g., how far or close each of the biologically active moieties M¹ and M² are), and spacing between the polymer backbone and the biological active moiety (e.g., the length of linkers off of the polymer backbone). This allows for constructing compounds with the biologically active moieties to facilitate an alkylation of guanine (G) of DNA such that the biological active moieties attached to the polymer backbone are located in a low energy position within the minor groove of DNA. Compounds disclosed in the present disclosure have multiple alkylating agents as the biological active moieties which are allowed to form interstrand and/or intrastrand DNA crosslinks resulting in greater DNA stabilization.

The biological active moieties can be attached to the polymer backbone via physiologically cleavable or non-cleavable linkers. The procedures described in the present disclosure provide the ability to selectively install the physiologically cleavable and/or non-cleavable linkers. This allows for synthesizing a compound which has both physiologically cleavable and non-cleavable linkers with one or more biological active moieties. In this regard, the biological active moieties can be cleaved sequentially depending on physiological conditions. Additionally, a compound with both a biological active moiety and a fluorescent moiety attached by physiologically cleavable and/or non-cleavable linkers can be synthesized.

Some embodiments of the present disclosure provide combinations of therapeutic agents, targeting moieties, and dye moieties (e.g., chromophores or fluorophores) that can be used for simultaneous targeting, treatment, and detection. The ease of coupling polymer-drug constructs to targeting agents such as antibodies, antibody fragments, proteins or other clinically interesting agents provides utility to a wide variety of interesting applications (e.g., surface chemistries, assay development, etc.). Accordingly, in some embodiments, M is a chromophore or fluorophore (e.g., FITC, 5-FAM, 6-FAM, and the like).

The compounds of certain embodiments also provide other desirable properties, including enhanced permeability and retention effects. In addition to providing necessary solubility characteristics, the chemical features of embodiments of the present compounds can be adjusted to modulate the compound's ability to permeate diseased cells/tissue and be retained within the same. These features allow effective delivery of biologically active agents by increasing permeation and increasing efficacy by enhancing retention.

Accordingly, it is understood that any embodiment of the compounds of Structures (I), (II), or (III), as set forth above, may be independently combined with other embodiments to form embodiments of the disclosure not specifically set forth above. It is understood that in the present description, combinations of substituents and/or variables of the depicted formulae are permissible only if such contributions result in stable compounds.

Accordingly, one embodiment provide a compound having the following structure (I):

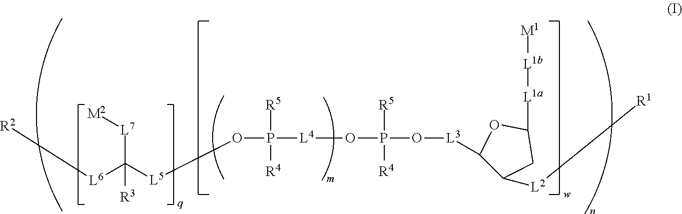

or a stereoisomer, pharmaceutically salt or tautomer thereof, wherein:
M¹ is, at each occurrence, independently absent, a moiety comprising a pyrrolobenzodiazepine, a minor groove binding agent or a fluorescent dye, provided that at least one occurrence of M¹ is a pyrrolobenzodiazepine;
M² is, at each occurrence, independently a moiety comprising a pyrrolobenzodiazepine, a minor groove binding agent or a fluorescent dye;
L^{1a} is, at each occurrence, independently a heteroarylene linker;
L^{1b} is, at each occurrence, independently H when M1 is absent or an alkylene, alkenylene, alkynylene, heteroalkylene, heteroalkenylene or heteroalkynylene linker when M¹ is a pyrrolobenzodiazepine or a fluorescent dye.
L², L³, L⁵, L⁶ and L⁷ are, at each occurrence, independently optional alkylene, alkenylene, alkynylene, heteroalkylene, heteroalkenylene or heteroalkynylene linkers;
L⁴ is, at each occurrence, independently an alkylene, alkenylene, alkynylene, heteroalkylene, heteroalkenylene or heteroalkynylene linker;
R¹ and R² are each independently H, OH, SH, alkyl, alkoxy, alkylether, heteroalkyl, —OP(═Rₐ)(R_{b})R_{c}, Q, or a protected form thereof, L′ or a minor groove binding agent;
R³ is, at each occurrence, independently H, alkyl or alkoxy;
R⁴ is, at each occurrence, independently O—, S—, OR_{d} or SR_{d};
R⁵ is, at each occurrence, independently oxo, thioxo or absent;
Rₐ is O or S;
R_{b} is OH, SH, O—, S—, ORₐ or SR_{d};
R_{c} is OH, SH, O—, S—, ORₐ, OL′, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether or thiophosphoalkylether;
R_{d} is a counter ion;
Q is, at each occurrence, independently a moiety comprising a reactive group, or protected form thereof, capable of forming a covalent bond with an analyte molecule, a targeting moiety, a solid support or a complementary reactive group Q′;
L′ is, at each occurrence, independently a linker comprising a covalent bond to Q, a linker comprising a covalent bond to a targeting moiety, a linker comprising a covalent bond to an analyte molecule, a linker comprising a covalent bond to a solid support, a linker comprising a covalent bond to a solid support residue, a linker comprising a covalent bond to a nucleoside or a linker comprising a covalent bond to a further compound of structure (I);
m is, at each occurrence, an integer of zero or greater;
n is an integer of one or greater; and
q and w are, at each occurrence, independently 0 or 1, provided that at least one of q or w is 1 at one occurrence.

The various linkers and substituents (e.g., M¹, M², Q, R¹, R², R³, R_{c}, L^{1a}, L^{1b}, L², L³, L⁴, L⁵, L⁶ and L⁷) in the compound of structure (I) are optionally substituted with one more substituent. For example, in some embodiments the optional substituent is selected to optimize the water solubility or other property of the compound of structure (I). In certain embodiments, each chromophore, alkyl, alkoxy, alkylether, heteroarylene, heteroalkyl, alkylene, alkenylene, alkynylene, heteroalkylene, heteroalkenylene, heteroalkynylene, alkoxyalkylether, phosphoalkyl, thiophosphoalkyl, phosphoalkylether and thiophosphoalkylether in the compound of structure (I) is optionally substituted with one more substituent selected from the group consisting of hydroxyl, alkoxy, alkylether, alkoxyalkylether, sulfhydryl, amino, alkylamino, carboxyl, phosphate, thiophosphate, phosphoalkyl, thiophosphoalkyl, phosphoalkylether and thiophosphoalkylether. In certain embodiments the optional substituent is —OP(═Rₐ)(R_{b})R_{c}, where Rₐ, R_{b} and R_{c} are as defined for the compound of structure (I).

In some embodiments, at least one occurrence of L^{1a} is an optionally substituted 5-9 membered heteroarylene linker. In certain embodiments, L^{1a} is a substituted 5-membered heteroarylene linker. In certain embodiments, L^{1a} is a substituted 6-membered heteroarylene linker. In certain embodiments, L^{1a} is a substituted 7-membered heteroarylene linker. In certain embodiments, L^{1a} is a substituted 8-membered heteroarylene linker. In certain embodiments, L^{1a} is a substituted 9-membered heteroarylene linker. In some related embodiments, L^{1a} is substituted with oxo, alkyl (e.g., methyl, ethyl, etc.) or combinations thereof. In certain embodiments, L^{1a} is, at each occurrence, unsubstituted. In some more specific embodiments, L^{1a} is, at each occurrence independently a pyrimidine. In some more specific embodiments, L^{1a} is, at each occurrence, independently cytosine or thymine. In some embodiments, L^{1a} is, at each occurrence, independently selected from cytosine and thymine such that the compound comprises a sequence of cytosine and thymine bases capable of triplex formation with a target DNA sequence. In some more specific embodiments, L^{1a} has the following structure:

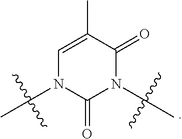 [SMILES: *n1cc(C)c(=O)n(*)c1=O]

In some embodiments, L^{1b} is L^{1b} is an alkylene linker. In certain embodiments, the alkylene linker of L^{1b} has odd number of carbon atoms. In some embodiments, the alkylene linker of L^{1b} is C₃ alkyl linker. In some embodiments, the alkylene linker of L^{1b} is C₅ alkyl linker. In some embodiments, the alkylene linker of L^{1b} has even number of carbon atoms. In some more specific embodiments, the alkylene linker of L^{1b} is C₂ alkyl linker. In some embodiments, the alkylene linker of L^{1b} is C₄ alkyl linker. In some embodiments, L^{1b} is, at each occurrence, independently an optional alkylene, alkenylene, alkynylene, heteroalkylene, heteroalkenylene, heteroalkynylene, alkyleneheteroarylenealkylene, alkyleneheterocyclylenealkylene, alkylenecarbocyclylenealkylene, heteroalkyleneheteroarylenealkylene, heteroalkyleneheterocyclylenealkylene, heteroalkylenecarbocyclylenealkylene, heteroalkyleneheteroaryleneheteroalkylene, heteroalkyleneheterocyclyleneheteroalkylene, heteroalkylenecarbocyclyleneheteroalkylene, alkyleneheteroaryleneheteroalkylene, alkyleneheterocyclyleneheteroalkylene, alkylenecarbocyclyleneheteroalkylene, heteroarylene, heterocyclylene, carbocyclylene, alkyleneheteroarylene, alkyleneheterocyclylene, heteroarylenealkylene, alkylenecarbocyclylene, carbocyclylenealkylene, heteroalkyleneheteroarylene, heteroalkyleneheterocyclylene, heteroaryleneheteroalkylene, heteroalkylenecarbocyclylene, carbocyclyleneheteroalkylene or heteroatomic linker. In some embodiments, L^{1b} is an optionally substituted heteroalkenylene linker.

In some embodiments, at least one occurrence of L^{1b} is substituted. In certain embodiments, L^{1b} is substituted at each occurrence. In some more specific embodiments, L^{1b} is substituted with oxo.

The linkers L^{1a}, L^{1b}, and L⁷ can be used as a point of attachment of the M¹ and M² moieties to the remainder of the compound. For example, in some embodiments a synthetic precursor to the compound of structure (I) is prepared, and the M¹ and M² moieties are attached to the synthetic precursor using any number of coupling methods known in the art. In more embodiments, for at least one occurrence of L^{1b} or L⁷, the functional group comprises an alkene, ester, amide, thioester, disulfide, carbocyclic, heterocyclic or heteroaryl group. In more embodiments, for at least one occurrence of L^{1b} or L⁷, the functional group comprises an alkene, ester, amide, thioester, thiourea, disulfide, carbocyclic, heterocyclic or heteroaryl group. In other embodiments, the functional group comprises an amide or thiourea.

In more specific embodiments, at least one occurrence of L⁷ comprises one of the following structures:

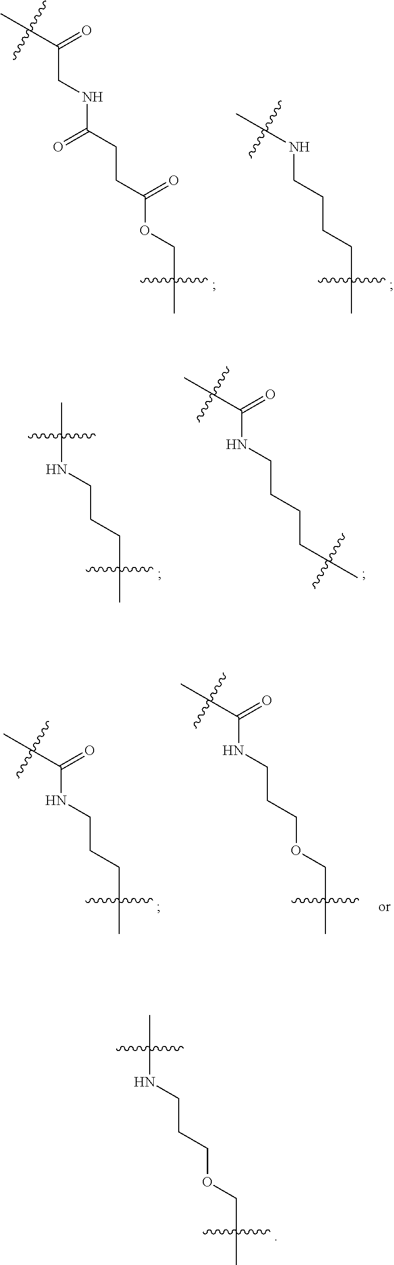

In some embodiments, L^{1b} has one of the following structures:

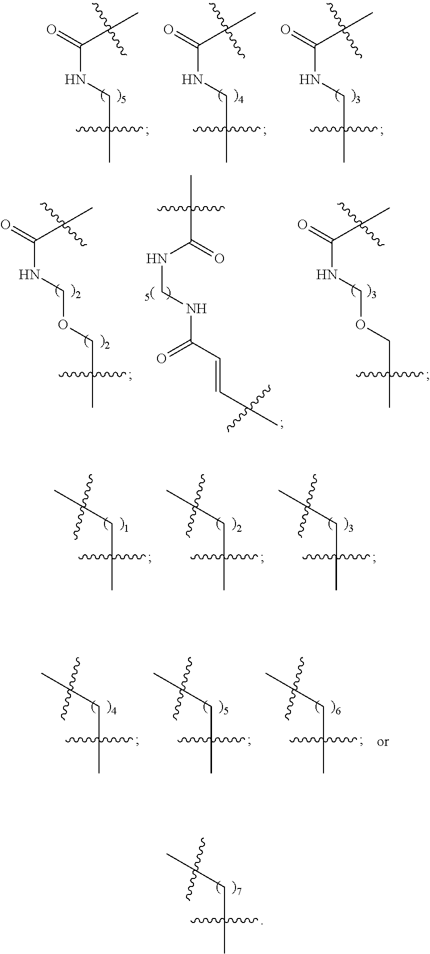

In some embodiments, at least one occurrence of L² is absent. In some more specific embodiments, L² is absent at each occurrence. In some specific embodiments, at least one occurrence of L² is heteroalkylene. In certain embodiments, at least one occurrence of L² comprises oxygen. In some embodiments, at least one occurrence of L² has the following structure:

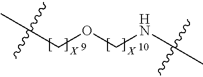

wherein:
x⁹ and x¹⁰ are each independently a integer greater than 0.

In some embodiments, x⁹ is 1, 2, 3, or 4. In certain embodiments, x¹⁰ is 2, 3, 4, or 5. In some specific embodiments, x⁹ is 1 or 2 and x¹⁰ is 2, 3, or 4. In certain specific embodiments, each occurrence of L² is heteroalkylene. In some more specific embodiments, each occurrence of L² comprises oxygen. In certain more specific embodiments, each occurrence of L² has the following structure:

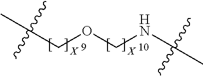

wherein:
x⁹ and x¹⁰ are each independently a integer greater than 0.

In some embodiments, x⁹ is 1, 2, 3, or 4. In certain embodiments, x¹⁰ is 2, 3, 4, or 5. In more specific embodiments, x⁹ is 1 or 2 and x¹⁰ is 2, 3, or 4. In certain other embodiments, at least one occurrence of L² comprises the following structure:

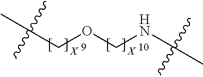

wherein:
x⁹ and x¹⁰ are each independently a integer greater than 0.

In certain embodiments, L² further comprises a physiologically cleavable linker. In more specific embodiments, at least one occurrence of L² comprises an amide bond, an ester bond, a phosphodiester bond, a disulfide bond, a double bond, a triple bond, an ether bond, a hydrazone, an amino acid sequence comprising one or more amino acid residues, a ketone, a diol, a cyano, a nitro, or combinations thereof. In more specific embodiments, at least one occurrence of L² comprises an amino acid sequence recognized by a sortase enzyme or cysteine protease. In certain embodiments, the amino acid sequence is Leu-Pro-X-Thr-Gly, wherein X is any amino acid residue. In more specific embodiments, at least one occurrence of L² comprises one of the following structures:

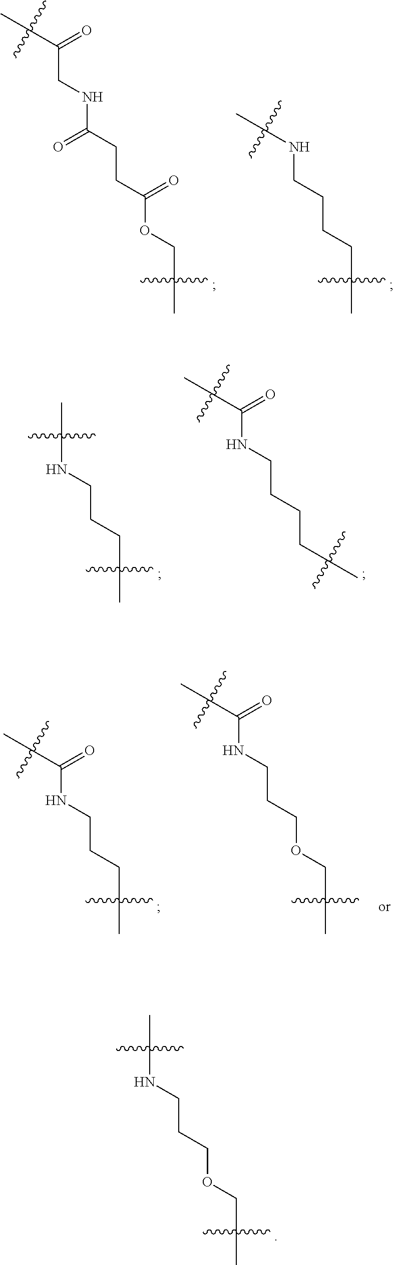

In certain embodiments, each occurrence of L² comprises an amide bond, an ester bond, a phosphodiester bond, a disulfide bond, a double bond, a triple bond, an ether bond, a hydrazone, an amino acid sequence, a ketone, a diol, a cyano, a nitro or combinations thereof. In some more specific embodiments, each occurrence of L² comprises one of the following structures:

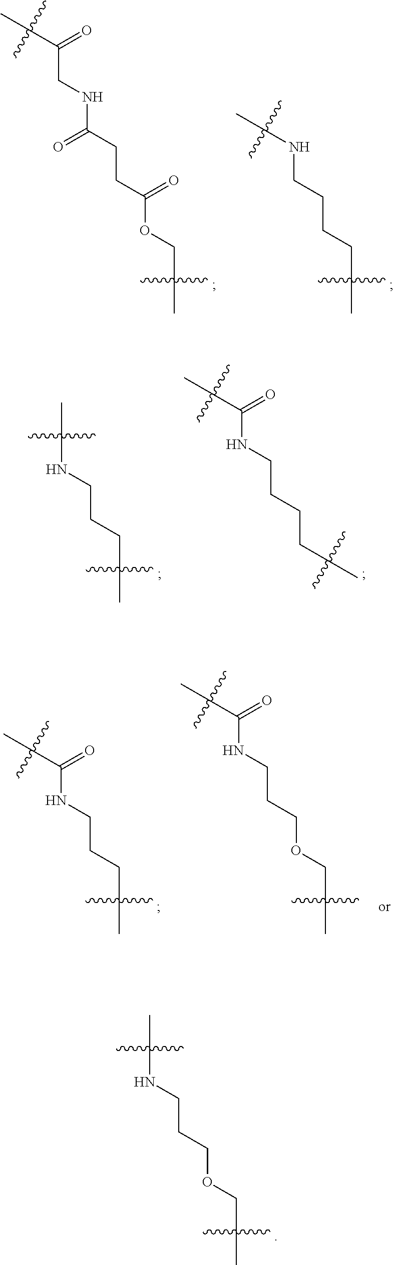

In some more specific embodiments, at least one occurrence of L² comprises one or more amino acid residues. In certain specific embodiments, at least one occurrence of L² comprises one or more amino acid residues selected from the group consisting of alanine, valine, and combinations thereof. In certain embodiments, at least one occurrence of L² comprises one of the following structures:

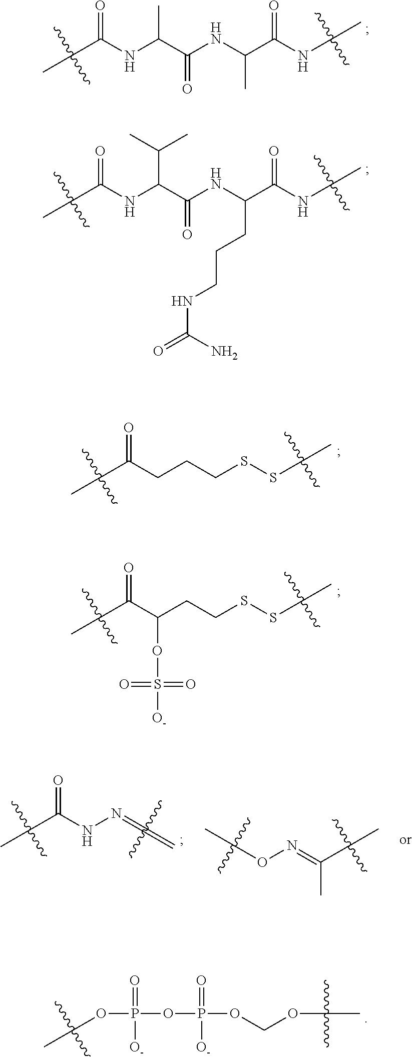

In some embodiments, each occurrence of L² comprises one or more amino acid residues. In certain embodiments, each occurrence of L² comprises one or more amino acid residues selected from the group consisting of alanine, valine, and combinations thereof. In some more specific embodiments, each occurrence of L² comprises one of the following structures:

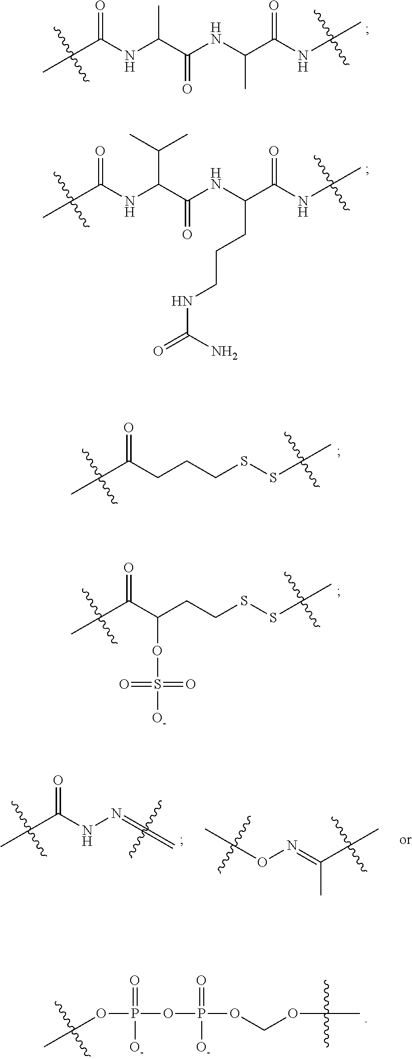

In more specific embodiments, at least one occurrence of L² has one of the following structures:

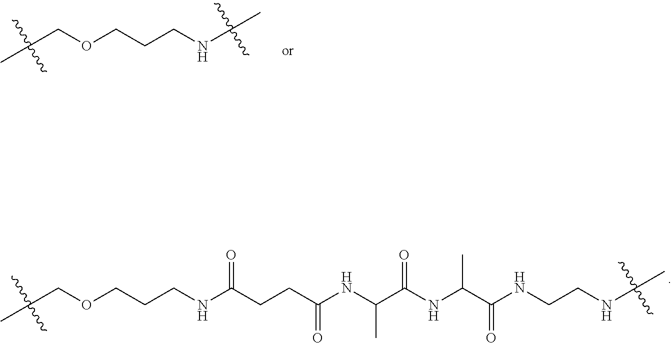 [SMILES: *COCCCN* ; *COCCCNC(=O)CCC(=O)NC(C)C(=O)NC(C)C(=O)NCCN*]

In some specific embodiments, each occurrence of L² has one of the following structures:

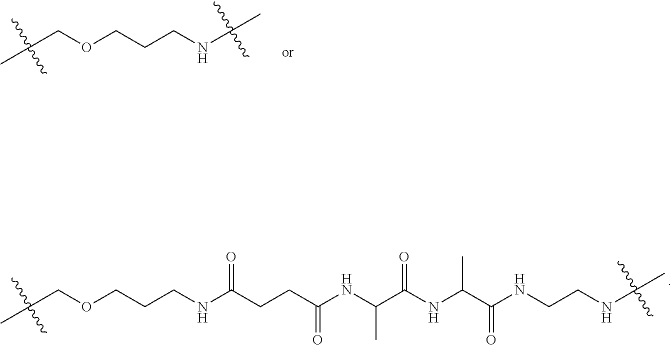 [SMILES: *COCCCN* ; *COCCCNC(=O)CCC(=O)NC(C)C(=O)NC(C)C(=O)NCCN*]

In some embodiments, at least one occurrence of L³ is an alkylene linker. In some embodiments, L³ is an alkylene linker at each occurrence. In certain embodiments, the alkylene linker is a methylene linker.

In some embodiments, at least one occurrence of L⁴ comprises alkylene oxide. In some embodiments, at least one occurrence of L⁵ or L⁶ is heteroalkylene. In some embodiments, at least one occurrence of L⁵ or L⁶ comprises alkylene oxide. In some more specific embodiments, the alkylene oxide of L⁵ or L⁶ is ethylene oxide. In some more specific embodiments, the ethylene oxide is polyethylene oxide. In certain embodiments, at least one occurrence of L⁵ or L⁶ is an alkylene linker (e.g., methylene). In some more specific embodiments, L⁵ or L⁶ is an alkylene linker at each occurrence (e.g., methylene). In certain embodiments, at least one occurrence of L⁵ is a heteroalkylene linker. In some more specific embodiments, L⁵ is a heteroalkylene linker at each occurrence. In some embodiments, at least one occurrence of L⁵ comprises alkylene oxide, for example, ethylene oxide (e.g., polyethylene oxide). In certain embodiments, at least one occurrence of L⁵ is an alkylene linker (e.g., methylene). In some more specific embodiments, L⁵ is an alkylene linker at each occurrence (e.g., methylene). In certain embodiments, at least one occurrence of L⁵ is absent. In some more specific embodiments, L⁵ is absent at each occurrence.

In certain embodiments, at least one occurrence of L⁶ is a heteroalkylene linker. In some more specific embodiments, L⁶ is a heteroalkylene linker at each occurrence. In some embodiments, at least one occurrence of L⁶ comprises alkylene oxide. In some of the foregoing embodiments, the alkylene oxide is ethylene oxide, for example, polyethylene oxide. In certain embodiments, at least one occurrence of L⁶ is an alkylene linker (e.g., methylene). In some more specific embodiments, L⁶ is an alkylene linker at each occurrence (e.g., methylene). In certain embodiments, at least one occurrence of L⁶ is absent. In some more specific embodiments, L⁶ is absent at each occurrence.

In certain embodiments, at least one occurrence of L⁵ or L⁶ comprises a phosphodiester moiety. In more specific embodiments, each occurrence of L⁵ or L⁶ comprises a phosphodiester moiety. In more embodiments, L², L³, L⁴ or L⁶ are, at each occurrence, independently C₁-C₆ alkylene, C₂-C₆ alkenylene or C₂-C₆ alkynylene.

In some embodiments, at least one occurrence of L⁵ is heteroalkylene. In some embodiments, L⁵ is heteroalkylene at each occurrence, for example, a heteroalkylene comprising one of the following structures:

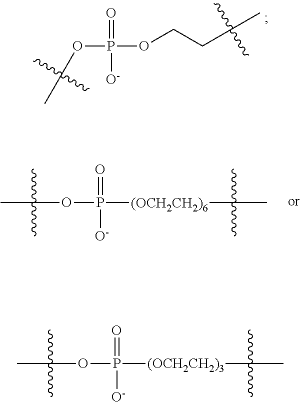

In some embodiments, at least one occurrence of L⁶ is heteroalkylene. In some embodiments, L⁶ is heteroalkylene at each occurrence, for example, a heteroalkylene comprising one of the following structures:

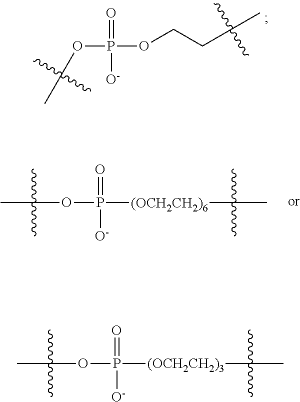

In some of the foregoing embodiments, a heteroalkylene (e.g., L³, L⁴, L⁵ or L⁶) comprises the following structure:

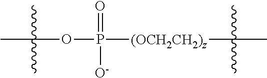

wherein
z is an integer ranging from 19 to 30. In some embodiments, z ranges from 19-28. In certain embodiments, the average z is 23. In some embodiments, the average z is 19, 20, 21, 22, 23, 24, 25, 26, 27, or 28.

In certain of the foregoing embodiments, the targeting moiety is an antibody or cell surface receptor antagonist. In other more specific embodiments of any of the foregoing compounds of structure (I), R¹ or R² has one of the following structures:

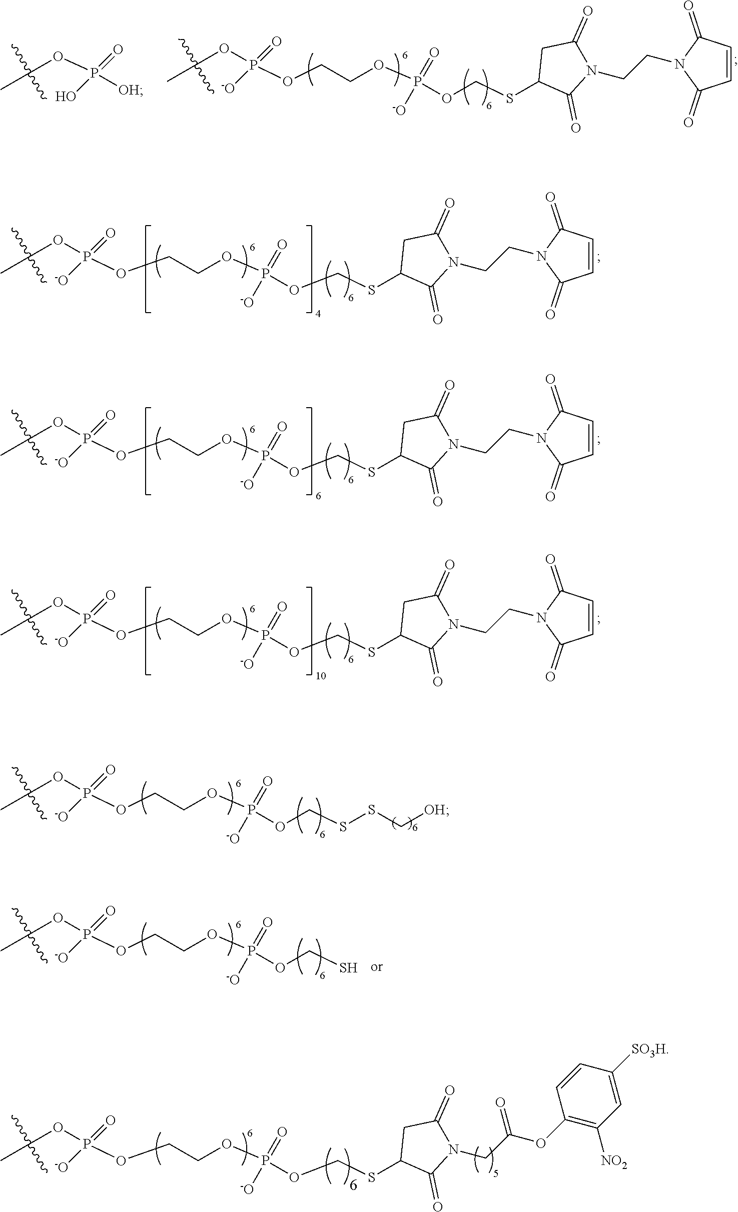

In other more specific embodiments of any of the foregoing compounds of structure (I), R¹ or R² has one of the following structures:

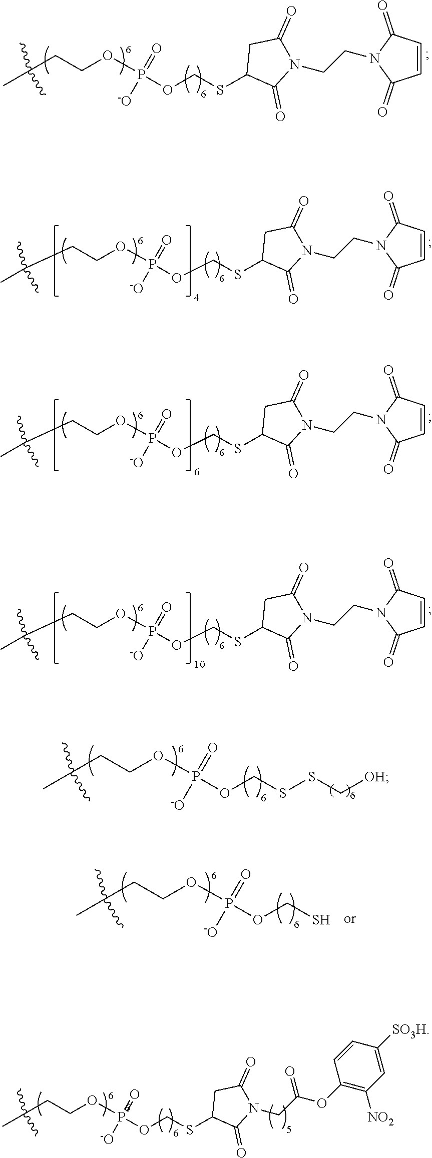

In other various embodiments, R¹ and R² are each independently OH or —OP(═Rₐ)(R_{b})R_{c}. In some different embodiments, R¹ or R² is OH or —OP(═Rₐ)(R_{b})R_{c}, and the other of R¹ or R² is Q or a linker comprising a covalent bond to Q.

In still more different embodiments of any of the foregoing compounds of structure (I), R¹ and R² are each independently —OP(═Rₐ)(R_{b})R_{C}. In some of these embodiments, R_{c} is OL′.

In other embodiments, R¹ and R² are each independently —OP(═Rₐ)(R_{b})OL′, and L′ is an alkylene or heteroalkylene linker to: Q, a targeting moiety, an analyte (e.g., analyte molecule), a solid support, a solid support residue, a nucleoside or a further compound of structure (I).

The linker L′ can be any linker suitable for attaching Q, a targeting moiety, an analyte (e.g., analyte molecule), a solid support, a solid support residue, a nucleoside or a further compound of structure (1) to the compound of structure (1). Advantageously certain embodiments include use of L′ moieties selected to increase or optimize water solubility of the compound. In certain embodiments, L′ is a heteroalkylene moiety. In some other certain embodiments, L′ comprises an alkylene oxide or phosphodiester moiety, or combinations thereof.

In some embodiments, L′ is a heteroalkylene linker to: Q, a targeting moiety, an analyte molecule, a solid support, a solid support residue, a nucleoside or a further compound of structure (I). In some more specific embodiments, L′ comprises an alkylene oxide or phosphodiester moiety, or combinations thereof. In certain embodiments, L′ has the following structure:

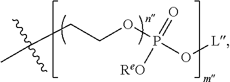

wherein:
m″ and n″ are independently an integer from 1 to 10;
R^{e} is H, an electron pair or a counter ion;
L″ is R^{e} or a direct bond or linkage to: Q, a targeting moiety, an analyte molecule, a solid support, a solid support residue, a nucleoside or a further compound of structure (I).

Certain embodiments of compounds of structure (I) can be prepared according to solid-phase synthetic methods analogous to those known in the art for preparation of oligonucleotides. Accordingly, in some embodiments, L′ is a linkage to a solid support, a solid support residue or a nucleoside. Solid supports comprising an activated deoxythymidine (dT) group are readily available, and in some embodiments can be employed as starting material for preparation of compounds of structure (I). Accordingly, in some embodiments R¹ or R² has the following structure:

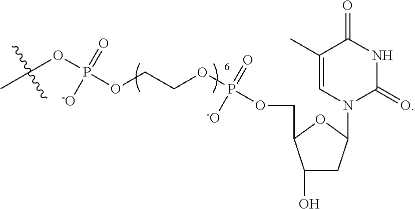

One of skill in the art will understand that the dT group depicted above is included for ease of synthesis and economic efficiencies only, and is not required. Other solid supports can be used and would result in a different nucleoside or solid support residue being present on L′, or the nucleoside or solid support residue can be removed or modified post synthesis.

In some embodiments, the targeting moiety is an antibody, cell surface receptor antagonist, or cell surface receptor antagonist. In some embodiments, the targeting moiety is a monoclonal antibody. In some more specific embodiments, the monoclonal antibody is Abciximab, Adalimumab, Alemtuzumab, Alirocumab, Avibactam, Basiliximab, Benralizumab, Bezlotoxumab, Blinatumomab, Brodalumab, Burosumab, Canakinumab, Caplacizumab, Certolizumab pegol, Daclizumab, Denosumab, Dupilumab, Eculizumab, Emicizumab, Erenumab, Evolocumab, Fremanezumab, Galcanezumab, Golimumab, Guselkumab, Ibalizumab, Idarucizumab, Infliximab, Itolizumab, Ixekizumab, Lanadelumab, Lokivetmab, Mepolizumab, Natalizumab, Obiltoxaximab, Ocrelizumab, Omalizumab, Palivizumab, Ranibizumab, Raxibacumab, Reslizumab, Rmab, Rovelizumab, Ruplizumab, Sarilumab, Secukinumab, Tildrakizumab, Thiomab, Tocilizumab, Ustekinumab, Vedolizumab, Abrilumab, Actoxumab, Aducanumab, Afasevikumab, Afelimomab, Anifrolumab, Anrukinzumab (IMA-638), Aselizumab, Atorolimumab, Bapineuzumab, BCD-100, Bertilimumab, Besilesomab, Biciromab, Bimagrumab, Bimekizumab, Birtamimab, Bleselumab, Blosozumab, Bococizumab, Brazikumab, Briakinumab, Brolucizumab, Carlumab, Carotuximab, Cedelizumab, Clazakizumab, Clenoliximab, Concizumab, Cosfroviximab, CR6261, Crenezumab, Crizanlizumab, Crotedumab, Depatuxizumab, mafodotin, Derlotuximab biotin, Dezamizumab, Diridavumab, Domagrozumab, Dusigitumab, Ecromeximab, Edobacomab, Efalizumab, Efungumab, Eldelumab, Elezanumab, Enokizumab, Eptinezumab, Erlizumab, Etrolizumab, Evinacumab, Exbivirumab, Fanolesomab, Faralimomab, Faricimab, Fasinumab, Felvizumab, Fezakinumab, Flanvotumab, Fletikumab, Flotetuzumab, Fontolizumab, Foravirumab, Frovocimab, Fulranumab, Gantenerumab, Gavilimomab, Gevokizumab, Gimsilumab, Gomiliximab, Gosuranemab, Ianalumab, Inclacumab, Inolimomab, Iomab-B, Keliximab, Lampalizumab, Landogrozumab, Larcaviximab, Lebrikizumab, Lenvervimab, Lerdelimumab, Letolizumab, Libivirumab, Ligelizumab, Lodelcizumab, Lulizumab pegol, Marstacimab, Mavrilimumab, Metelimumab, Mirikizumab, Motavizumab, Muromonab CD3, Nebacumab, Nemolizumab, NEOD001, Nirsevimab, Odulimomab, Olendalizumab, Olokizumab, OMS721, Opicinumab, Orticumab, Otelixizumab, Otilimab, Oxelumab, Ozanezumab, Ozoralizumab, Pagibaximab, Panobacumab, Pascolizumab, Pateclizumab, PDR001, Perakizumab, Pexelizumab, Placulumab, Plozalizumab, Ponezumab, Porgaviximab, Prasinezumab, Priliximab, PRO 140, Quilizumab, Rafivirumab, Ralpancizumab, Ranevetmab, Ravagalimab, Ravulizumab, Refanezumab, Regavirumab, Relatlimab, Rinucumab, Risankizumab, Roledumab, Romosozumab, Rontalizumab, SA237, Satralizumab, Sevirumab, SHP647, Sifalimumab, Simtuzumab, Siplizumab, Sirukumab, Solanezumab, Sonepcizumab, Spartalizumab, Stamulumab, Sulesomab, Suptavumab, Sutimlimab, Suvizumab, Suvratoxumab, Tadocizumab, Talizumab, Tamtuvetmab, Tanezumab, Tefibazumab, Telimomab aritox, Teneliximab, Teplizumab, Teprotumumab, Tezepelumab, Tibulizumab, Toralizumab, Tralokinumab, Trevogrumab, Tuvirumab, Ulocuplumab, Urtoxazumab, Varisacumab, Vepalimomab, Vesencumab, Visilizumab, Vobarilizumab, Zolimomab aritox, trastuzumab, gemtuzumab, brentuximab, vorsetuzumab, lorvotuzumab, cantuzumab, bivatuzumabor inotuzumab, or vadastuximab.

In some embodiments, the analyte molecule is a nucleic acid, amino acid or a polymer thereof. In some embodiments, the analyte molecule is an enzyme, receptor, receptor ligand, antibody, glycoprotein, aptamer or prion. In some embodiments, the targeting moiety is an antibody or cell surface receptor antagonist. In further some embodiments, the solid support is a polymeric bead or non-polymeric bead.

In some embodiments, n is an integer from 1 to 100. In some more specific embodiments, n is an integer from 1 to 10. In some embodiments, m is an integer from 7 to 12. In certain more specific embodiments, m is an integer from 3 to 6.

In some embodiments, at least one occurrence of R³ is H. In some embodiments, R⁴ is, at each occurrence, oxo. In some embodiments, R⁵ is, at each occurrence, independently OH, O⁻ or OR_{d}. In still other embodiments of any of the compounds of structure (I), R⁵ is, at each occurrence, independently OH, O⁻ or ORₐ. It is understood that “OR_{d}” and “SR_{d}” are intended to refer to O⁻ and S⁻ associated with a cation. For example, the disodium salt of a phosphate group may be represented as:

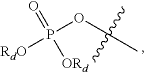

where R_{d} is sodium (Na⁺).

In other embodiments of any of the compounds of structure (I), at least one occurrence of R⁴ is oxo. In other embodiments of any of the compounds of structure (I), R⁴ is, at each occurrence, oxo.

In some embodiments, the compound has the following structure (Ia):

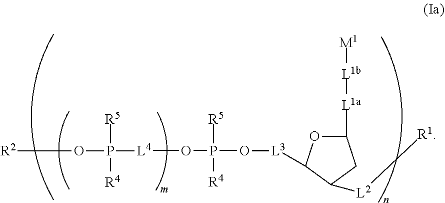

In more specific embodiments, the compound has the following structure (Ib):

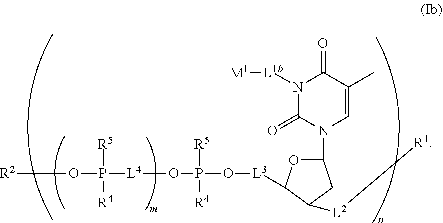

wherein:
L^{1b} is, at each occurrence, independently an optionally alkylene or an optionally heteroalkylene linker.

In some embodiments, the compound has the following structure (Ic):

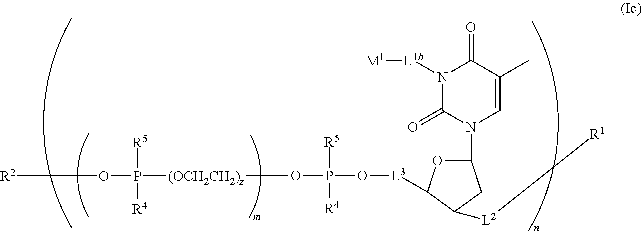

wherein:
   z is an integer from 1 to 100.

In some embodiments, M¹ has one of the following structures:

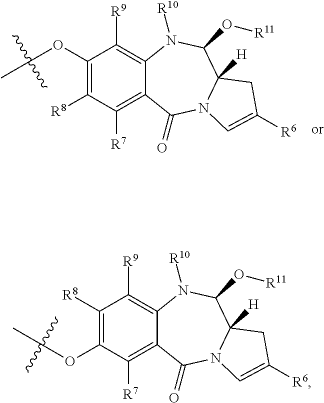

wherein:
R⁶ is, at each occurrence, independently H, CH═CHCONH2, alkyl, alkoxy, heteroalkyl, heteroalkoxy, alkylether, alkoxyalkylether, cycloalkyl, heterocyclyl, aryl, or heteroaryl;
R⁷, R⁸, and R⁹ are, at each occurrence, independently H, OH, OR_{f}, SH, SR_{f}, NH₂, NHR_{f}, NR_{f}R_{g}, alkyl, alkoxy, alkylether, or heteroalkyl;
R¹⁰ is, at each occurrence, independently a nitrogen protecting group or H;
R¹¹ is, at each occurrence, independently an oxygen protecting group, alkyl, or H; and
R_{f} and R_{g} are, at each occurrence, independently alkyl, heterocyclyl, or aryl.

In some embodiments, M², R¹ or R² comprises a minor groove binding agent. In some more specific embodiments, the minor groove binding agent has one of the following structures:

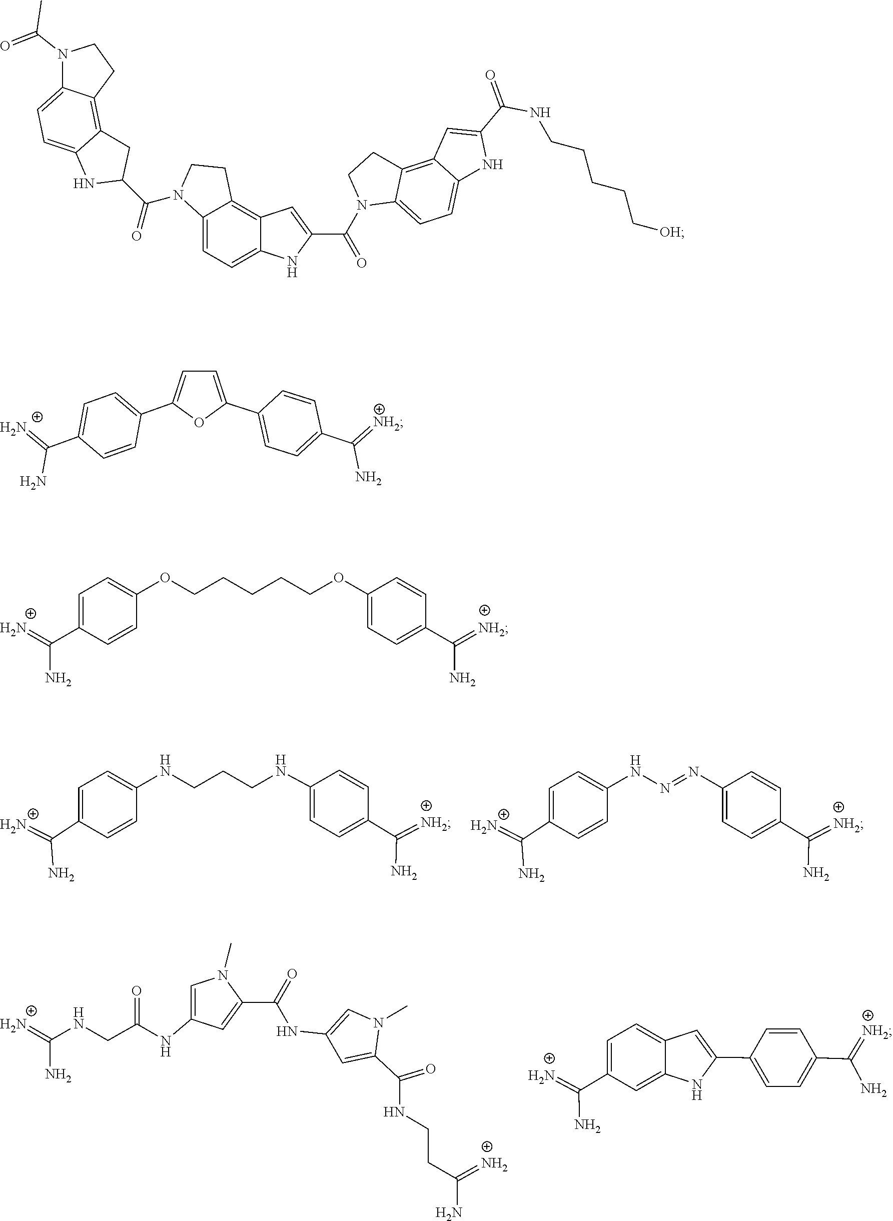

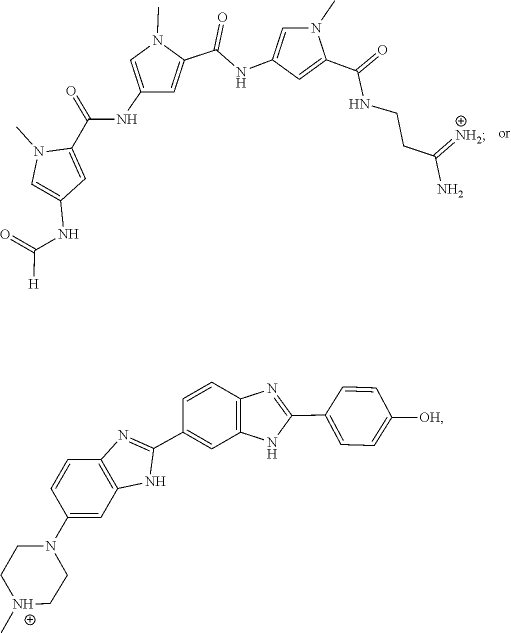

wherein one substitutable position of the minor groove binding agent is covalently bound to the remainder of the compound via an optional linker.

In certain embodiments, the compound has one of the following structures (Id) or (Ie):

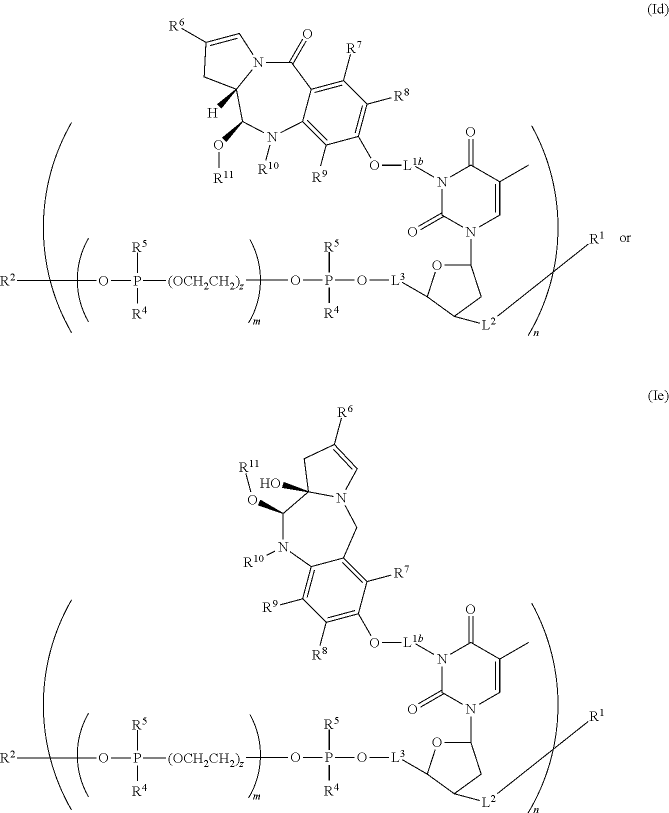

wherein:
   R⁶ is, at each occurrence, independently an alkyl;
   R⁷ and R⁹ are, at each occurrence, H;
   R⁸ is, at each occurrence, independently OR_{f};
R¹⁰ is, at each occurrence, independently a nitrogen protecting group;
R¹¹ is, at each occurrence, independently an oxygen protecting group; and
R_{f} is an alkyl.

In some embodiments, at least one occurrence of L³ is an alkylene linker. In more specific embodiments, L³ or is an alkylene linker at each occurrence. In certain embodiments, the alkylene linker is a methylene linker.

In some embodiments, at least one occurrence of L² is absent. In more specific embodiments, L² is absent at each occurrence.

In still other embodiments, Q is, at each occurrence, independently a moiety comprising a reactive group capable of forming a covalent bond with an analyte molecule or a solid support. In other embodiments, Q is, at each occurrence, independently a moiety comprising a reactive group capable of forming a covalent bond with a complementary reactive group Q′. For example, in some embodiments, Q′ is present on a further compound of structure (I) (e.g., in the R¹ or R² position), and Q and Q′ comprise complementary reactive groups such that reaction of the compound of structure (I) and the further compound of structure (I) results in covalently bound dimer of the compound of structure (I). Multimer compounds of structure (I) can also be prepared in an analogous manner and are included within the scope of embodiments of the disclosure.

The type of Q group and connectivity of the Q group to the remainder of the compound of structure (I) is not limited, provided that Q comprises a moiety having appropriate reactivity for forming the desired bond.

In certain embodiments, Q is a moiety which is not susceptible to hydrolysis under aqueous conditions, but is sufficiently reactive to form a bond with a corresponding group on an analyte molecule or solid support (e.g., an amine, azide or alkyne).

Certain embodiments of compounds of structure (I) comprise Q groups commonly employed in the field of bioconjugation. For example in some embodiments, Q comprises a nucleophilic reactive group, an electrophilic reactive group or a cycloaddition reactive group. In some more specific embodiments, Q comprises a sulfhydryl, disulfide, activated ester, isothiocyanate, azide, alkyne, alkene, diene, dienophile, acid halide, sulfonyl halide, phosphine, α-haloamide, biotin, amino or maleimide functional group. In some embodiments, the activated ester is an N-succinimide ester, imidoester or polyflourophenyl ester. In other embodiments, the alkyne is an alkyl azide or acyl azide.

The Q groups can be conveniently provided in protected form to increase storage stability or other desired properties, and then the protecting group removed at the appropriate time for conjugation with, for example, a targeting moiety or analyte. Accordingly, Q groups include “protected forms” of a reactive group, including any of the reactive groups described above and in the Table 1 below. A “protected form” of Q refers to a moiety having lower reactivity under predetermined reaction conditions relative to Q, but which can be converted to Q under conditions, which preferably do not degrade or react with other portions of the compound of structure (I). One of skill in the art can derive appropriate protected forms of Q based on the particular Q and desired end use and storage conditions. For example, when Q is SH, a protected form of Q includes a disulfide, which can be reduce to reveal the SH moiety using commonly known techniques and reagents.

Exemplary Q moieties are provided in Table I below.

TABLE 1
Exemplary Q Moieties

| Structure | Class |
|---|---|
| 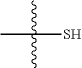 [SMILES: *S] | Sulfhydryl |
| 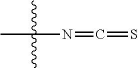 [SMILES: *N=C=S] | Isothiocyanate |
| 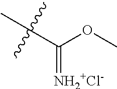 | Imidoester |
| 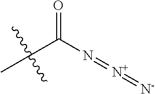 [SMILES: *C(=O)N=[N+]=[N-]] | Acyl Azide |
| 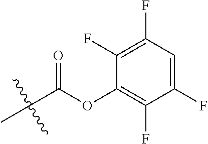 [SMILES: *C(=O)Oc1c(F)c(F)cc(F)c1F] | Activated Ester |
| 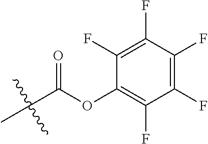 [SMILES: *C(=O)Oc1c(F)c(F)c(F)c(F)c1F] | Activated Ester |
| 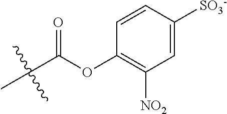 | Activated Ester |
| 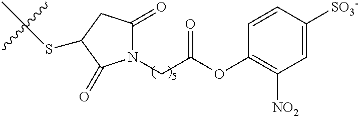 | Activated Ester |
| 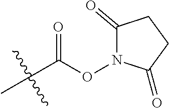 [SMILES: *C(=O)ON1C(=O)CCC1=O] | Activated Ester |
| 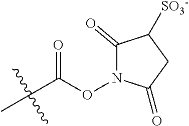 | Activated Ester |
| 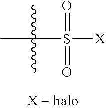 | Sulfonyl halide |
| 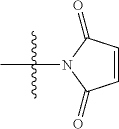 [SMILES: *N1C(=O)C=CC1=O] | Maleimide |
| 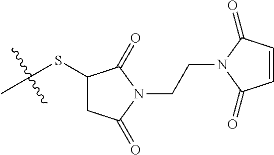 [SMILES: *SC1CC(=O)N(CCN2C(=O)C=CC2=O)C1=O] | Maleimide |
| 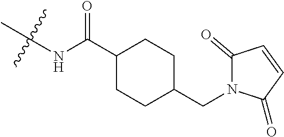 [SMILES: *NC(=O)C1CCC(CN2C(=O)C=CC2=O)CC1] | Maleimide |
| 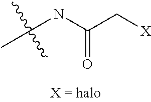 | α-haloimide |
| 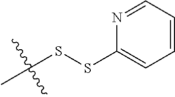 [SMILES: *SSc1ccccn1] | Disulfide |
| 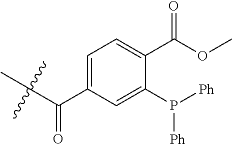 [SMILES: *C(=O)c1ccc(C(=O)OC)c(P(c2ccccc2)c2ccccc2)c1] | Phosphine |
| 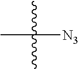 [SMILES: *N=[N+]=[N-]] | Azide |
| 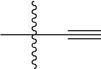 [SMILES: *C#C] | Alkyne |
| 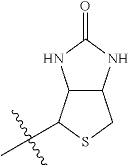 [SMILES: *C1SCC2NC(=O)NC21] | Biotin |
| 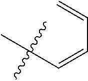 [SMILES: */C=C\C=C] | Diene |
| 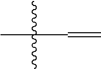 [SMILES: *C=C] | Alkene/dienophile |
| 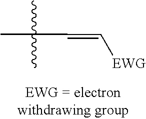 | Alkene/dienophile |
| 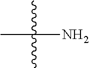 [SMILES: *N] | Amino |

It should be noted that in some embodiments, wherein Q is SH, the SH moiety will tend to form disulfide bonds with another sulfhydryl group, for example on another compound of structure (I). Accordingly, some embodiments include compounds of structure (I), which are in the form of disulfide dimers, the disulfide bond being derived from SH Q groups.

Also included within the scope of certain embodiments are compounds of structure (I), wherein one, or both, of R¹ and R² comprises a linkage to a further compound of structure (I). For example, wherein one or both of R¹ and R² are —OP(═Rₐ)(R_{b})R_{c}, and R_{c} is OL′, and L′ is a linker comprising a covalent bond to a further compound of structure (I). Such compounds can be prepared by preparing a first compound of structure (I) having for example about 10 “M¹” and/or “M²” moieties (i.e., n=10) and having an appropriate “Q” for reaction with a complementary Q′ group on a second compound of structure (I). In this manner, compounds of structure (I), having any number of “M¹” and/or “M²” moieties, for example 100 or more, can be prepared without the need for sequentially coupling each monomer. Exemplary embodiments of such compounds of structure (I) have the following structure (I′):

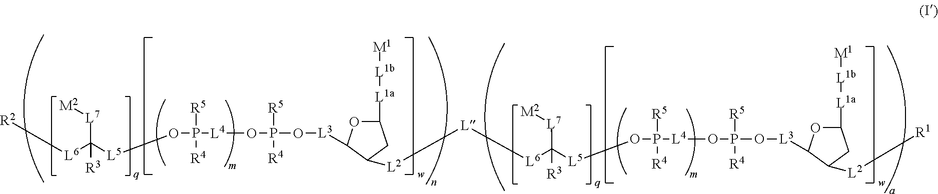

wherein:
each occurrence of R¹, R², R³, R⁴, R⁵, L^{1a}, L^{1b}, L², L³, L⁴, L⁵, L⁶, L⁷, M¹, M¹, q, m, w and n are independently as defined for a compound of structure (I);
L″ is a linker comprising a functional group resulting from reaction of a
Q moiety with a corresponding Q′ moiety; and
α is an integer greater than 1, for example from 1 to 100, or 1 to 10.

Compounds of structure (I′) are derivable by those of ordinary skill in the art, for example by dimerizing or polymerizing compounds of structure (I) provided herein.

In other embodiments, the Q moiety is conveniently masked (e.g., protected) as a disulfide moiety, which can later be reduced to provide an activated Q moiety for binding to a desired analyte molecule or targeting moiety. For example, the Q moiety may be masked as a disulfide having the following structure:

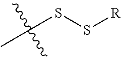

wherein R is an optionally substituted alkyl group. For example, in some embodiments, Q is provided as a disulfide moiety having the following structure:

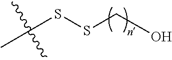

where n is an integer from 1 to 10.

In some other embodiments, one of R¹ or R² is OH or —OP(═Rₐ)(R_{b})R_{c}, and the other of R¹ or R² is a linker comprising a covalent bond to an analyte molecule or a linker comprising a covalent bond to a solid support. For example, in some embodiments the analyte molecule is a nucleic acid, amino acid or a polymer thereof. In other embodiments, the analyte molecule is an enzyme, receptor, receptor ligand, antibody, glycoprotein, aptamer or prion. In some embodiments, the targeting moiety is an antibody or cell surface receptor antagonist. In still different embodiments, the solid support is a polymeric bead or non-polymeric bead.

The fluorescence intensity or the effectiveness toward alkylation of DNA can also be tuned by selection of different values of n. In certain embodiments, n is an integer from 1 to 100. In other embodiments, n is an integer from 1 to 10. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

The fluorescence or the effectiveness toward alkylation of DNA may also be tuned by selection of values for m. The values for m has the ability to control the spacing between neighboring M¹ or M². In certain embodiments, m is an integer from 1 to 100. In other embodiments, m is an integer from 7 to 12. In some embodiments, m is an integer from 20 to 26. In some embodiments, m is an integer from 3 to 6. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11.

In certain embodiments, at least one occurrence of M¹ or M² is a nitrogen mustard, a nitrosourea, a tetrazine, an aziridine, a cisplatin or cisplatin derivative, or a non-classical alkylating agent. In some embodiments, at least one occurrence of M¹ or M² has the following structure:

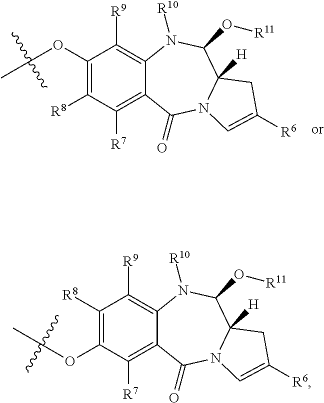

wherein:
R⁴″ is alkoxy, haloalkyl, alkyl, an optionally substituted aryl or an optionally substituted aralkyl;
R⁶ is, at each occurrence, independently H, CH═CHCONH2, alkyl, alkoxy, heteroalkyl, heteroalkoxy, alkylether, alkoxyalkylether, cycloalkyl, heterocyclyl, aryl, or heteroaryl;
R⁷, R⁸, and R⁹ are, at each occurrence, independently H, OH, OR_{f}, SH, SR_{f}, NH₂, NHR_{f}, NR_{f}R_{g}, alkyl, alkoxy, alkylether, or heteroalkyl;
R¹⁰ is, at each occurrence, independently a nitrogen protecting group or H;
R¹¹ is, at each occurrence, independently an oxygen protecting group, alkyl, or H; and
R_{f} and R_{g} are, at each occurrence, independently alkyl, heterocyclyl, or aryl.

In certain embodiments, at least one occurrence of M¹ or M² has one of the following structures:

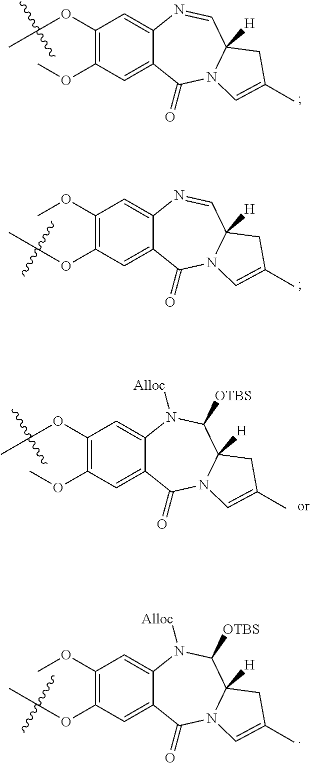

M¹ and M² are selected based on the desired alkylating properties. In some embodiments, M¹ and M² are the same at each occurrence; however, it is important to note that each occurrence of M¹ and M² need not be an identical M¹ and M², and certain embodiments include compounds wherein M¹ and M² are not the same at each occurrence. For example, in some embodiments each M¹ and M² are not the same and the different M¹ and M² moieties are selected to have different alkylating agents. Exemplary M¹ and M² moieties can be appropriately selected by one of ordinary skill in the art based on the desired end use. Exemplary M¹ and M² moieties for alkylation of DNA include pyrrolo benzo diazepine (PBD).

Further, in some embodiments, alkylating agents are protected by protecting groups such as an allyloxycarbonyl group (-Alloc) and a tert-butyl dimethyl silyl ether (-TBS) in order to survive during the DNA synthesis cycle. Alloc protecting group can be easily cleaved by a palladium catalyst, for example, Pd(PPh₃)₄ with PhSiH₃ to afford the corresponding amine. TBS protecting group can be cleaved by a fluoride source such as tetra-n-butylammonium fluoride (TBAF) to afford the corresponding alcohol. Once the deprotections are complete, PBD moiety becomes active with alkylating capability. In this regard, PBD moiety is protected with protecting groups until the DNA synthesis cycle is completed. Then, the deprotections allow for converting the protected PBD moiety which is inactive in alkylating into the deprotected PBD moiety which is active in alkylating.

In some embodiments, at least one occurrence of M¹ and M² moieties are an alkylating agent, an antimetabolite, a microtubule inhibitor, a topoisomerase inhibitor, or a cytotoxic antibiotic. In more specific embodiments, each occurrence of M is an alkylating agent, an antimetabolite, a microtubule inhibitor, a topoisomerase inhibitor, or a cytotoxic antibiotic. In some embodiments, at least one occurrence of M is an alkylating agent, an antimetabolite, a microtubule inhibitor, or a topoisomerase inhibitor. In more specific embodiments, each occurrence of M is an alkylating agent, an antimetabolite, a microtubule inhibitor, or a topoisomerase inhibitor. In certain embodiments, at least one occurrence of M is a nitrogen mustard, a nitrosourea, a tetrazine, an aziridine, a cisplatin or cisplatin derivative, or a non-classical alkylating agent. In more specific embodiments, at least one occurrence of M is mechlorethamine, cyclophosphamide, melphalan, chlorambucil, ifosfamide, busulfan, N-nitroso-N-methylurea (MNU), carmustine (BCNU), lomustine (CCNU), semustine (MeCCNU), fotemustine, streptozotocin, dacarbazine, mitozolomide, temozolomide, thiotepa, mytomycin, diaziquone (AZQ), cisplatin, carboplatin, oxaliplatin, procarbazine, or hexamethylmelamine. In some embodiments, at least one occurrence of M is an anti-folate, a fluoropyrimidines, a deoxynucleoside analogue, or a thiopurine. In certain embodiments, at least one occurrence of M is methotrexate, pemetrexed, fluorouracil, capecitabine, cytarabine, gemcitabine, decitabine, azacitidine, fludarabine, nelarabine, cladribine, clofarabine, pentostatin, thioguanine, and mercaptopurine. In some specific embodiments, at least one occurrence of M is an auristatin, a Vinca alkaloid, or a taxane. In certain specific embodiments, at least one occurrence of M is auristatin F, auristatin E, vincristine, vinblastine, vinorelbine, vindesine, vinflunine, paclitaxel, docetaxel, etoposide, or teniposide. In some more specific embodiments, at least one occurrence of M is irinotecan, SN 38, topotecan, camptothecin, doxorubicin, mitoxantrone, teniposide. novobiocin, merbarone, or aclarubicin. In certain more specific embodiments, at least one occurrence of M is an anthracycline or a bleomycin. In some embodiments, at least one occurrence of M is doxorubicin, daunorubicin, epirubicin, idarubicin, pirarubicin, aclarubicin, or mitoxantrone. In some embodiments, at least one occurrence of M is auristatin F, monomethyl auristatin F, monomethyl auristatin E, paciltaxol, SN-38, calicheamicin, anthramycin, abbeymycin, chicamycin, DC-81, mazethramycin, neothramycin A, neothramycin B, porothramycin prothracarcin, sibanomicin, sibiromycin, tomamycin, mertansine, emtansine, irinotecan, camptothecin, topotecan, silatecan, cositecan, Exatecan, Lurtotecan, gimatecan, Belotecan, and Rubitecan. In some embodiments, each occurrence of M is auristatin F, monomethyl auristatin F, monomethyl auristatin E, paciltaxol, SN-38, calicheamicin, anthramycin, abbeymycin, chicamycin, DC-81, mazethramycin, neothramycin A, neothramycin B, porothramycin prothracarcin, sibanomicin, sibiromycin, tomamycin, mertansine, emtansine, irinotecan, camptothecin, topotecan, silatecan, cositecan, Exatecan, Lurtotecan, gimatecan, Belotecan, and Rubitecan.

M¹ or M² may be attached to the remainder of the molecule from any position (i.e., atom) on M¹ or M², respectively. One of skill in the art will recognize means for attaching M¹ or M² to the remainder of molecule. For example, M¹ or M² may be attached to the remainder of the molecule through nitrogen of diazepine, oxygen of diazepine or phenyl ring, or carbon of pyrrolidine ring.

In some specific embodiments, the compound is a compound selected from Table 2A and 2B. The compounds in Table 2A and 2B are prepared according to the procedures set forth in the Examples.

TABLE 2A
Exemplary Compounds of Structure (Ia)-(Ie)

| No. | Structure |
|---|---|
| I-1 | 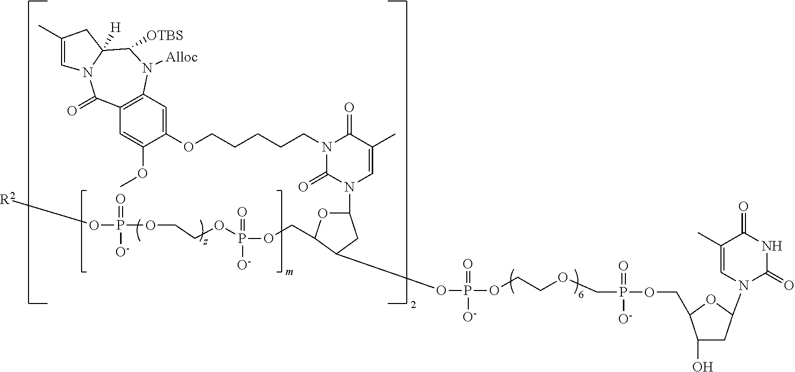 |
| | 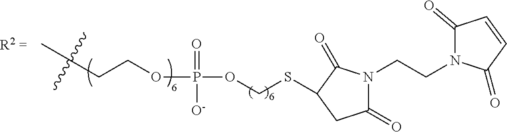 |
| I-2 | 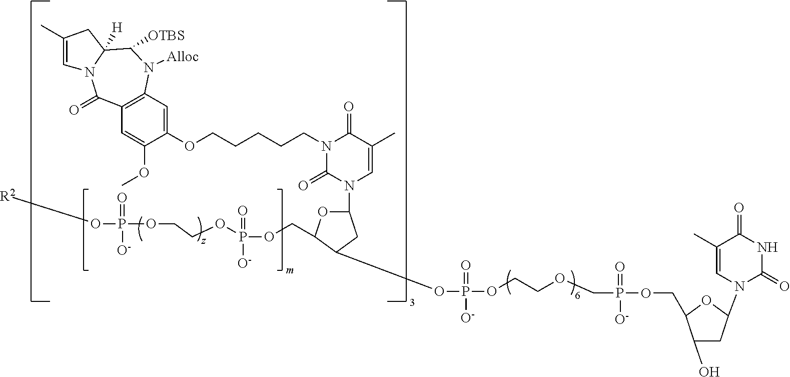 |
| | 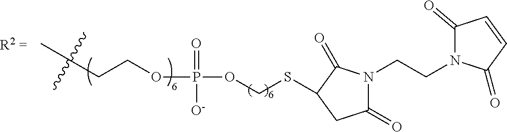 |
| I-3 | 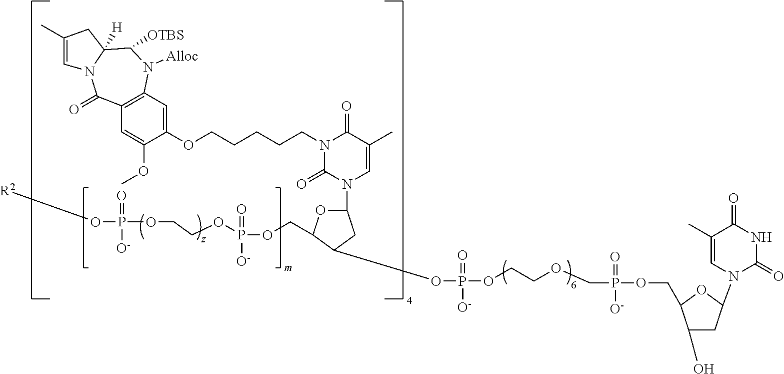 |
| | 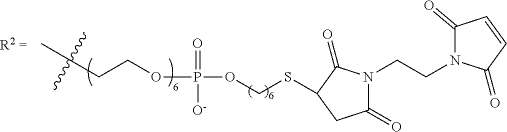 |
| I-4 | 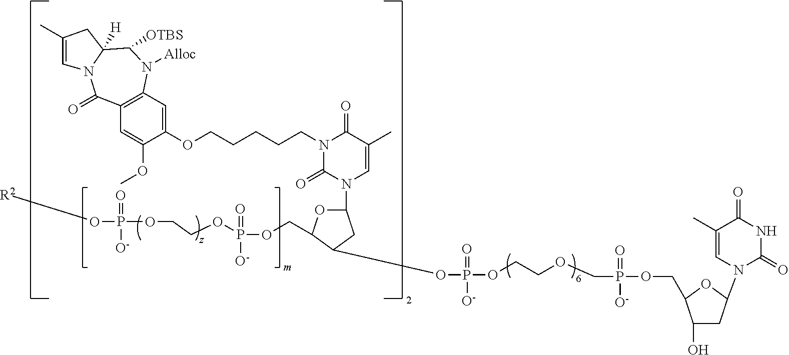 |
| | 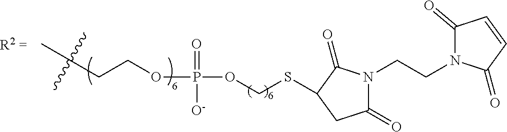 |
| I-5 | 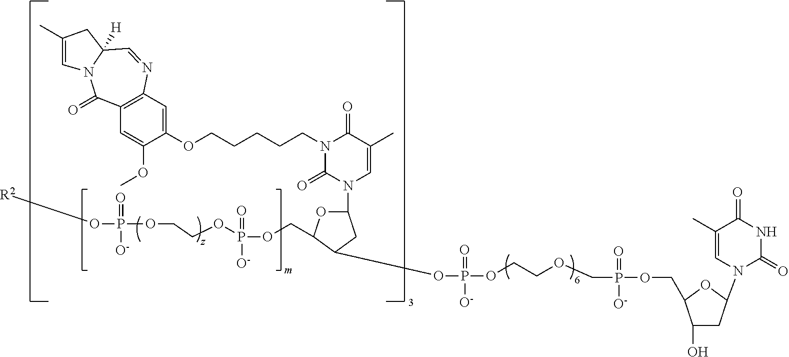 |
| | 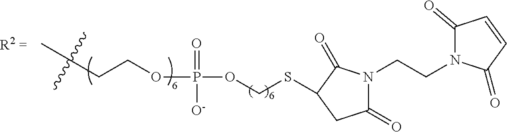 |
| I-6 | 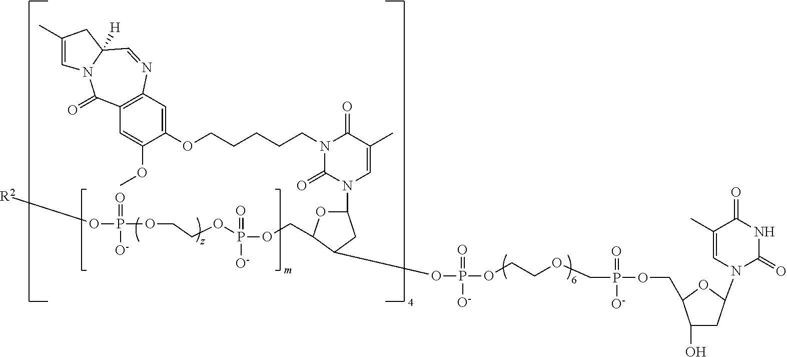 |
| | 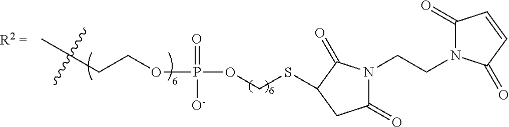 |
| I-7 | 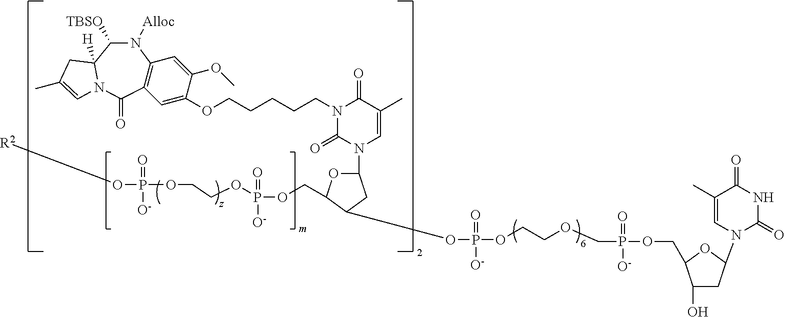 |
| | 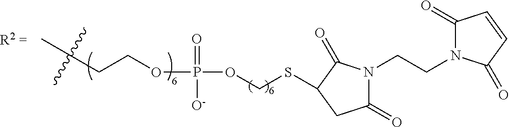 |
| I-8 | 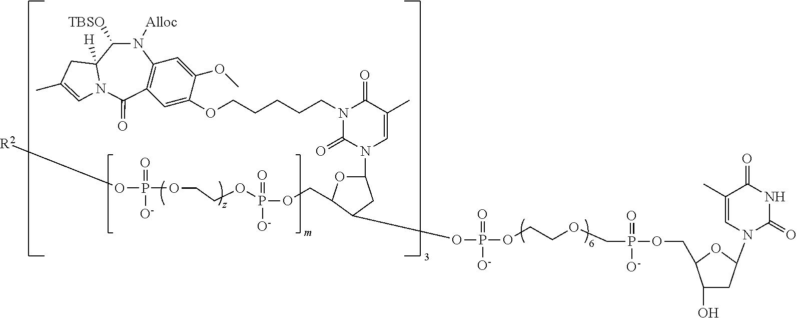 |
| | 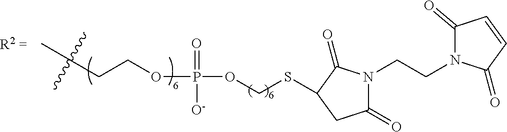 |
| I-9 | 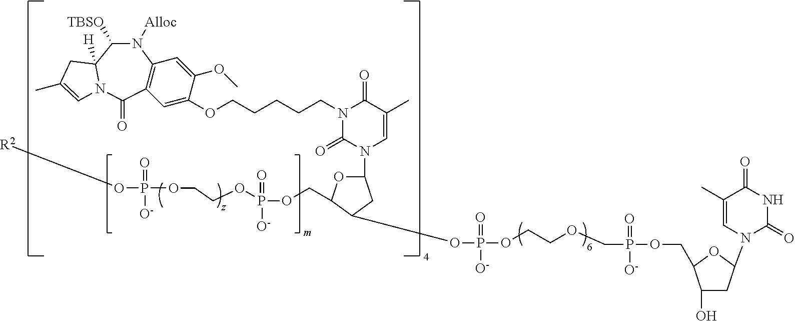 |
| | 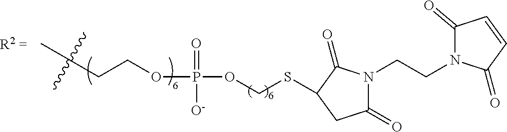 |
| I-10 | 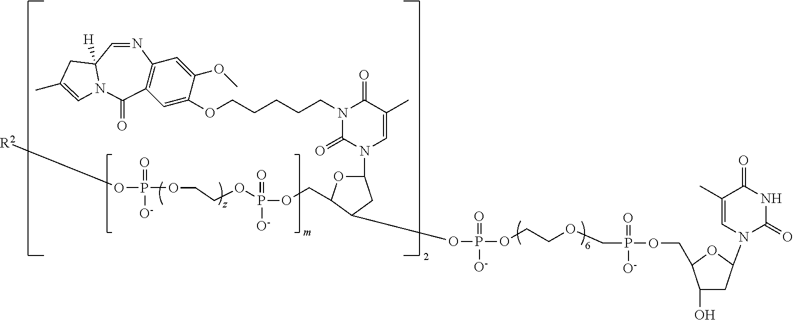 |
| | 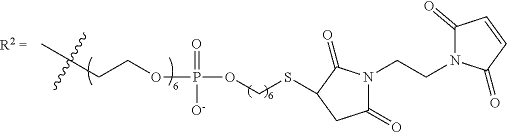 |
| I-11 | 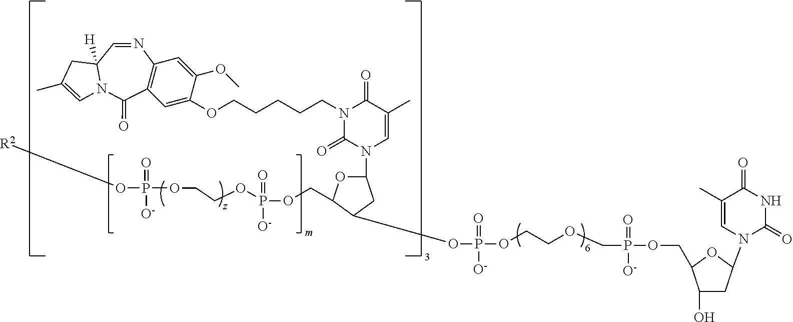 |
| | 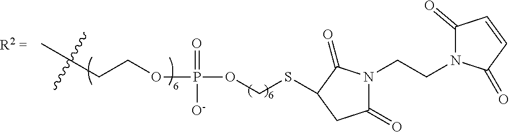 |
| I-12 | 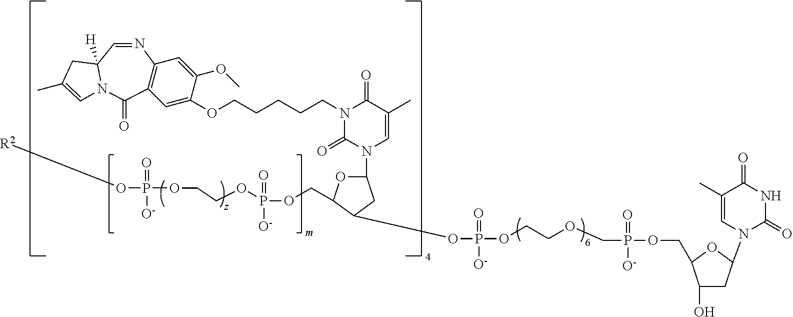 |
| | 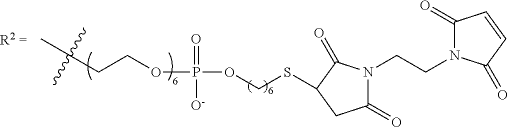 |

TABLE 2B
Exemplary Compounds of Structure (I)

| No. | Structure |
|---|---|
| I-13 | 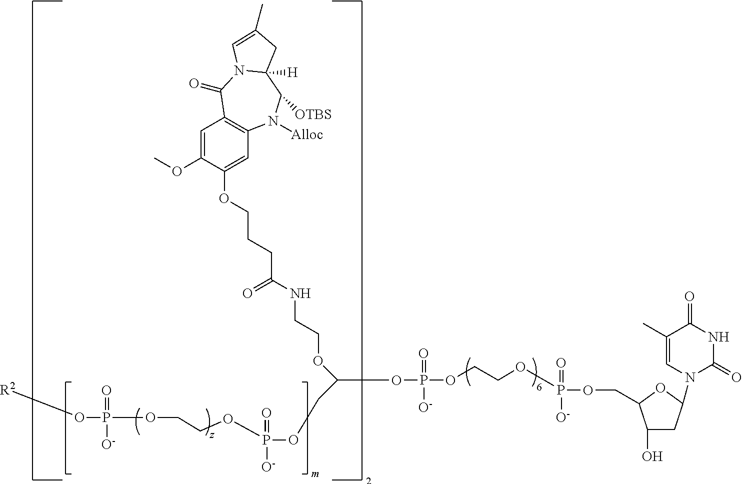 |
| | 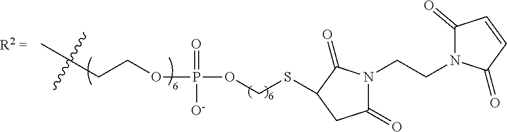 |
| I-14 | 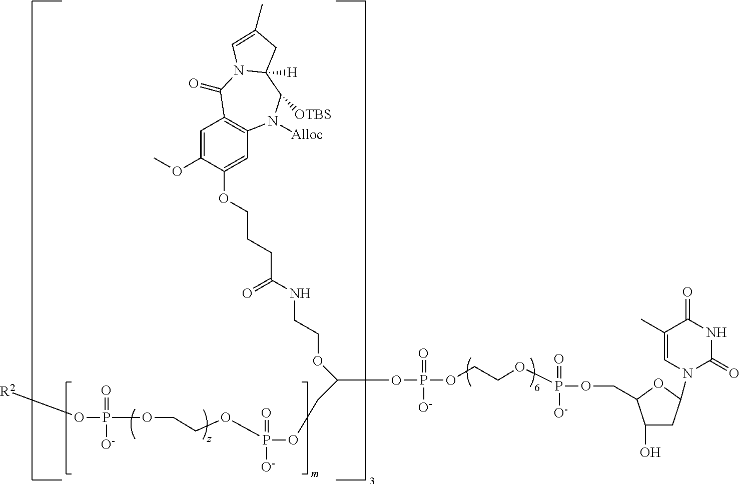 |
| | 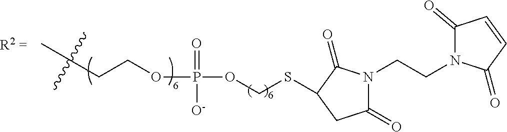 |
| I-15 | 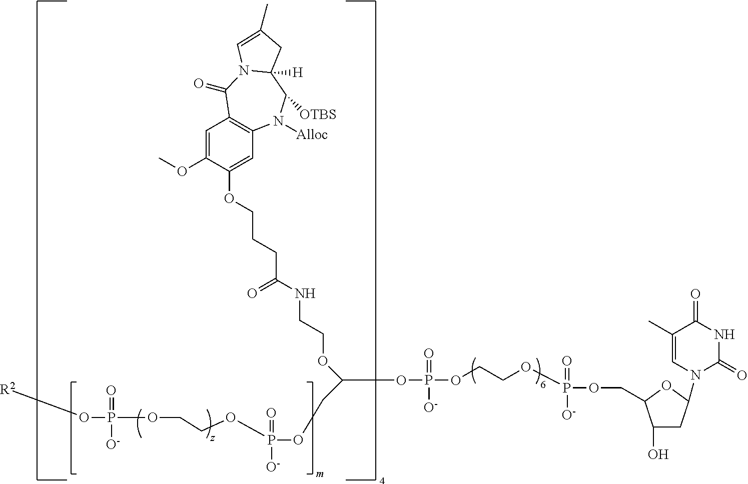 |
| | 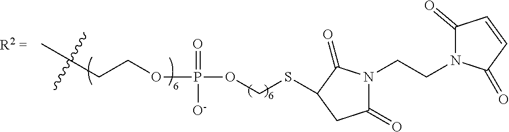 |
| I-16 | 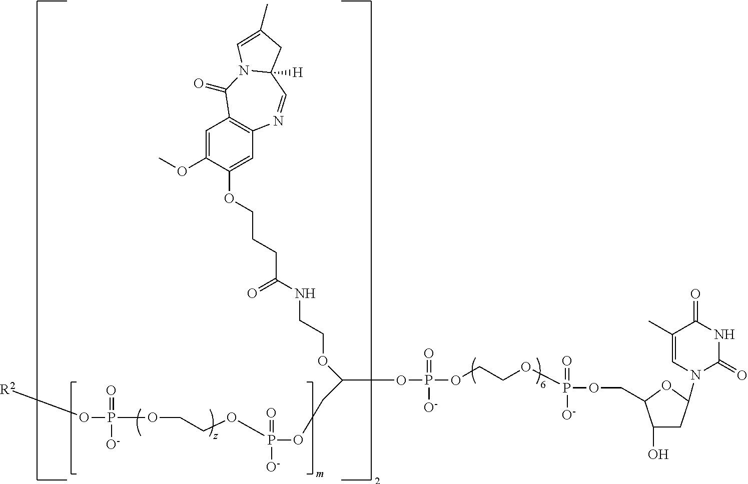 |
| | 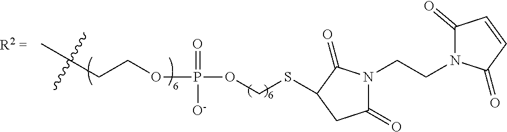 |
| I-17 | 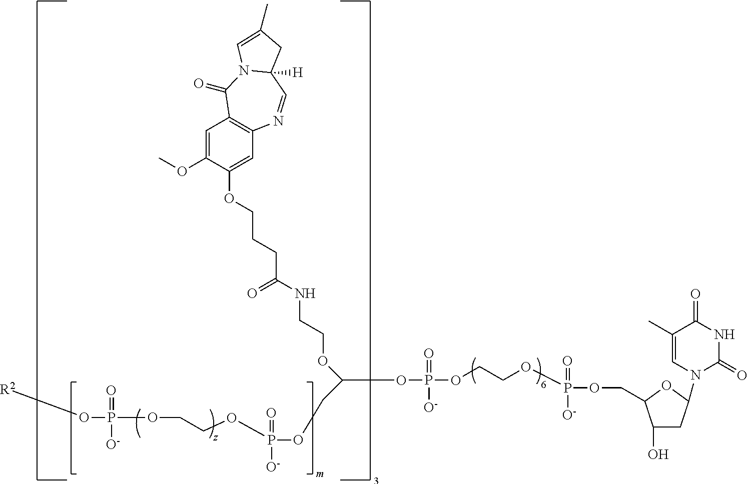 |
| | 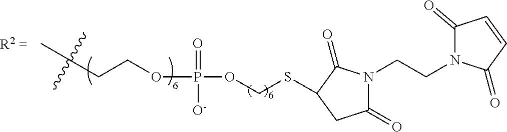 |
| I-18 | 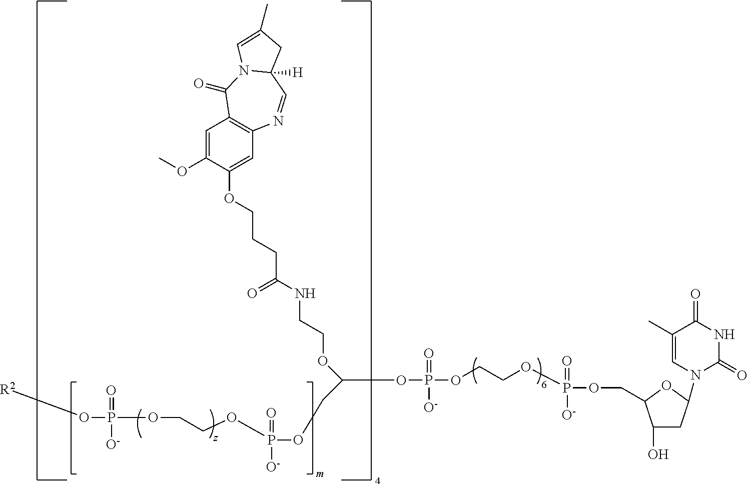 |
| | 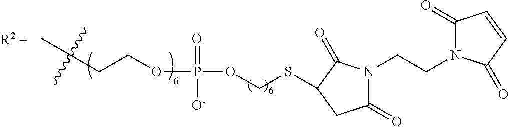 |
| I-19 | 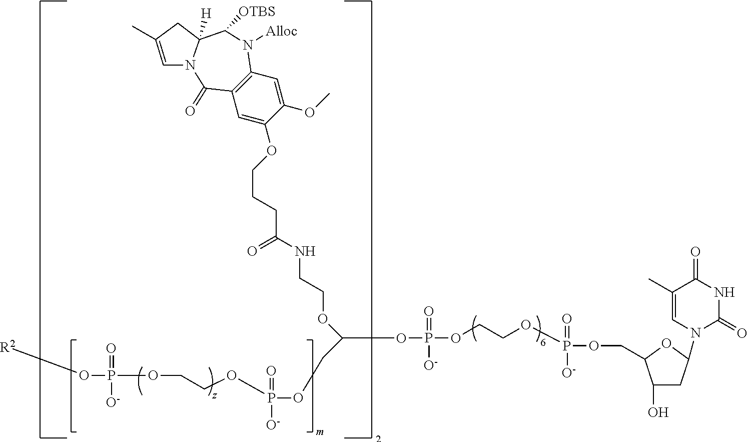 |
| | 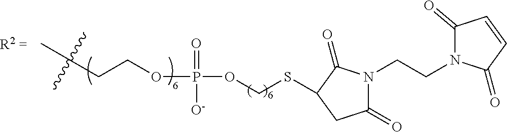 |
| I-20 | 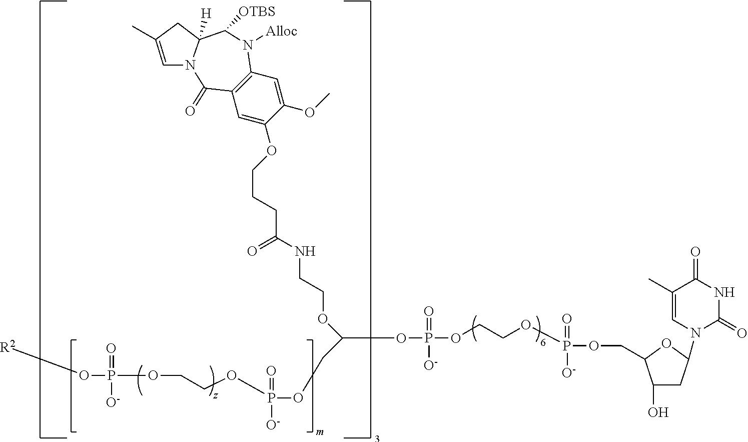 |
| | 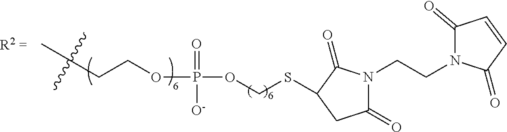 |
| I-21 | 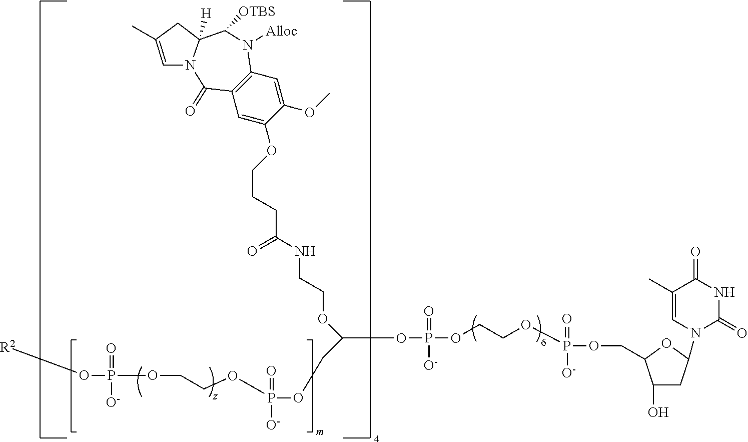 |
| | 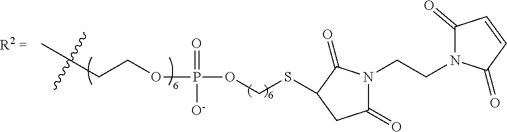 |
| I-22 | 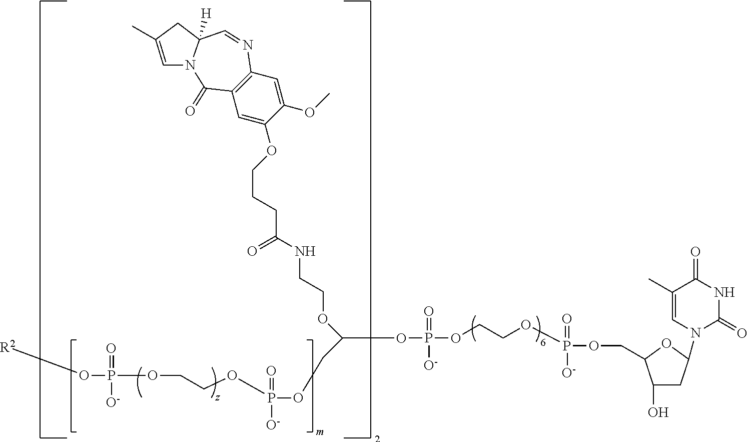 |
| | 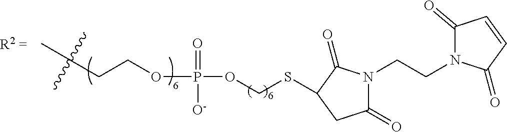 |
| I-23 | 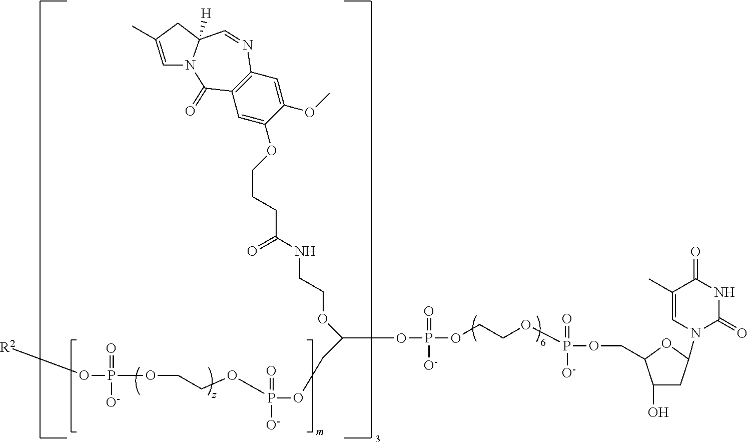 |
| | 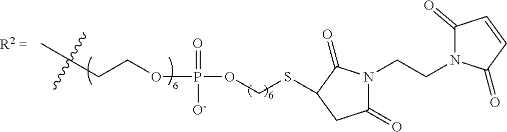 |
| I-24 | 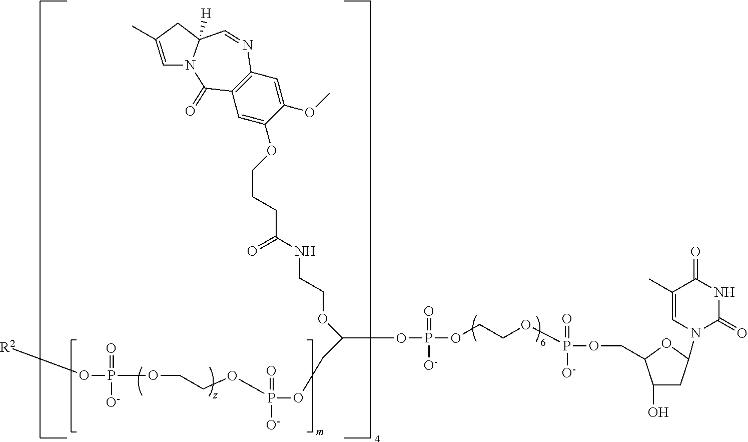 |
| | 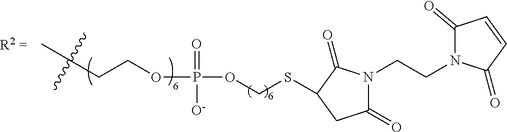 |

In some embodiments, M¹ and M² are, at each occurrence, independently a fluorescent or colored moiety. Any fluorescent and/or colored moiety may be used, for examples those known in the art and typically employed in colorimetric, UV, and/or fluorescent assays may be used. Examples of M¹ and M² moieties which are useful in various embodiments of the disclosure include, but are not limited to: Xanthene derivatives (e.g., fluorescein, rhodamine, Oregon green, eosin or Texas red); Cyanine derivatives (e.g., cyanine, indocarbocyanine, oxacarbocyanine, thiacarbocyanine or merocyanine); Squaraine derivatives and ring-substituted squaraines, including Seta, SeTau, and Square dyes; Naphthalene derivatives (e.g., dansyl and prodan derivatives); Coumarin derivatives; oxadiazole derivatives (e.g., pyridyloxazole, nitrobenzoxadiazole or benzoxadiazole); Anthracene derivatives (e.g., anthraquinones, including DRAQ5, DRAQ7 and CyTRAK Orange); Pyrene derivatives such as cascade blue; Oxazine derivatives (e.g., Nile red, Nile blue, cresyl violet, oxazine 170); Acridine derivatives (e.g., proflavin, acridine orange, acridine yellow); Arylmethine derivatives: auramine, crystal violet, malachite green; and Tetrapyrrole derivatives (e.g., porphin, phthalocyanine or bilirubin). Other exemplary M¹ and M² moieties include: Cyanine dyes, xanthate dyes (e.g., Hex, Vic, Nedd, Joe or Tet); Yakima yellow; Redmond red; tamra; Texas Red and Alexa Fluor® dyes.

In still other embodiments of any of the foregoing, M¹ and M² each occurrence independently comprises three or more aryl or heteroaryl rings, or combinations thereof, for example four or more aryl or heteroaryl rings, or combinations thereof, or even five or more aryl or heteroaryl rings, or combinations thereof. In some embodiments, M¹ and M² each occurrence independently comprises six aryl or heteroaryl rings, or combinations thereof. In further embodiments, the rings are fused. For example in some embodiments, M¹ and M² each occurrence independently comprises three or more fused rings, four or more fused rings, five or more fused rings, or even six or more fused rings. In certain embodiments, the fluorescent dye of M¹ and M² are, at each occurrence, independently a dimethylaminostilbene, quinacridone, fluorophenyl-dimethyl-BODIPY, his-fluorophenyl-BODIPY, acridine, terrylene, sexiphenyl, porphyrin, benzopyrene, (fluorophenyl-dimethyl-difluorobora-diaza-indacene)phenyl, (bis-fluorophenyl-difluorobora-diaza-indacene)phenyl, quaterphenyl, bi-benzothiazole, ter-benzothiazole, bi-naphthyl, bi-anthracyl, squaraine, squarylium, 9, 10-ethynylanthracene or ter-naphthyl moiety. In some embodiments, the fluorescent dye of M¹ and M² are, at each occurrence, independently p-terphenyl, perylene, azobenzene, phenazine, phenanthroline, acridine, thioxanthrene, chrysene, rubrene, coronene, cyanine, perylene imide, or perylene amide or derivative thereof. In some embodiments, the fluorescent dye of M¹ and M² are, at each occurrence, independently a coumarin dye, resorufin dye, dipyrrometheneboron difluoride dye, ruthenium bipyridyl dye, energy transfer dye, thiazole orange dye, polymethine or N-aryl-1,8-naphthalimide dye. In some embodiments, the fluorescent dye of M¹ and M² are, at each occurrence, independently pyrene, perylene, perylene monoimide or 6-FAM or derivative thereof. In some specific embodiments, the fluorescent dye of M¹ and M², at each occurrence, independently has one of the following structures:

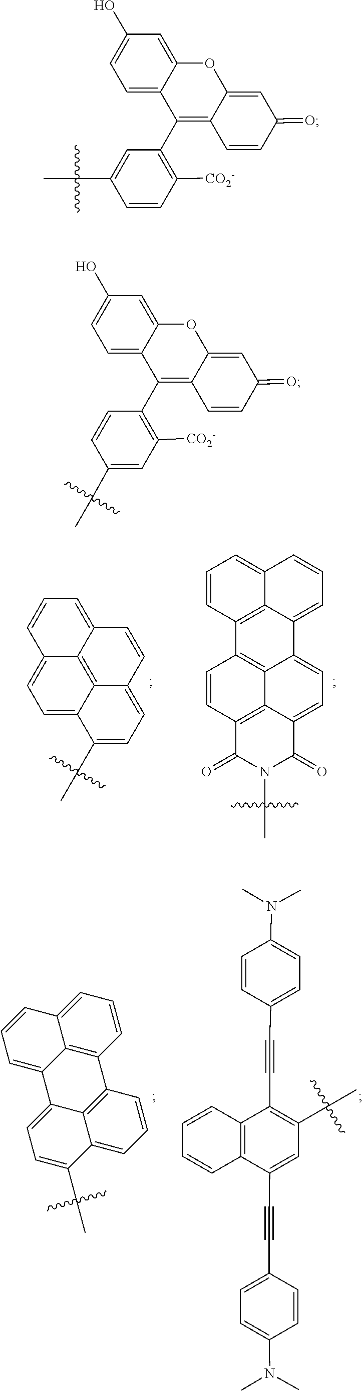

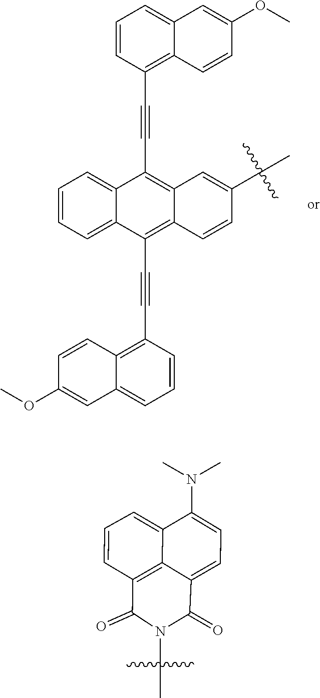

Although M¹ and M² moieties comprising carboxylic acid groups are depicted in the anionic form (CO₂—) above, one of skill in the art will understand that this will vary depending on pH, and the protonated form (i.e., —CO₂H) is included in various embodiments.

In some specific embodiments, the compound is a compound selected from Table 3A and 3B. The compounds in Table 3A and 3B are prepared according to the procedures set forth in the Examples.

TABLE 3A
Exemplary Compounds of Structure (I)

| No. | Structure |
|---|---|
| I-25 | 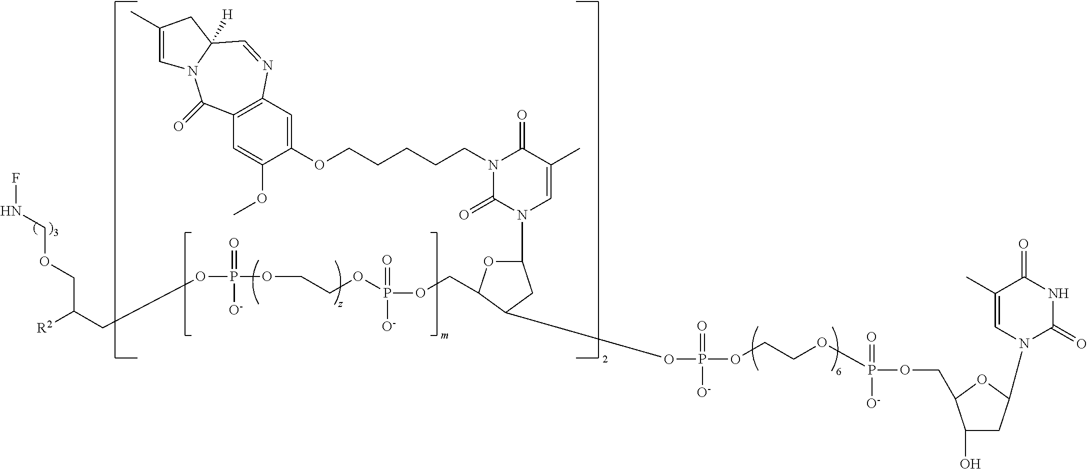 |
| | 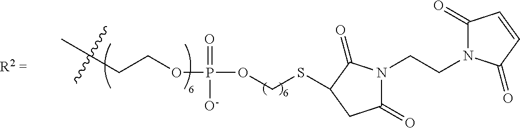 |
| I-26 | 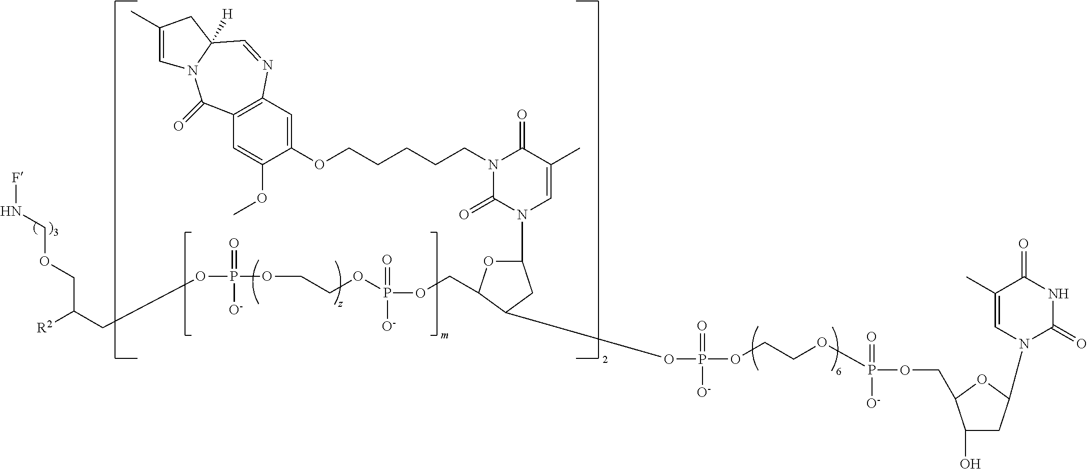 |
| | 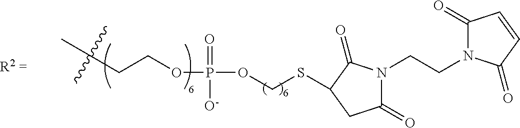 |
| I-27 | 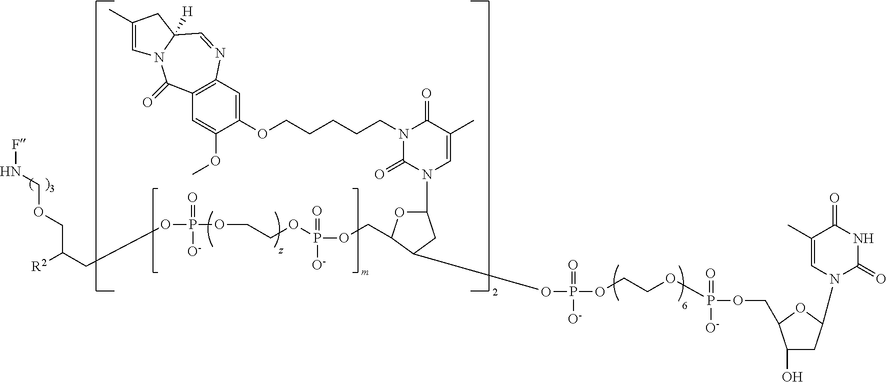 |
| | 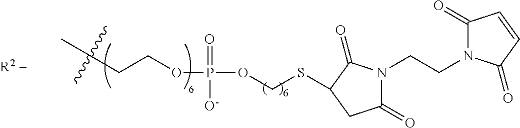 |
| I-28 | 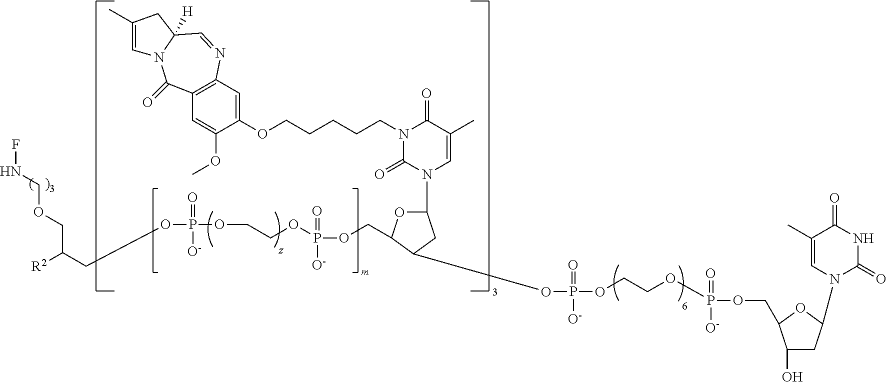 |
| | 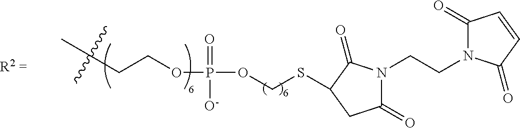 |
| I-29 | 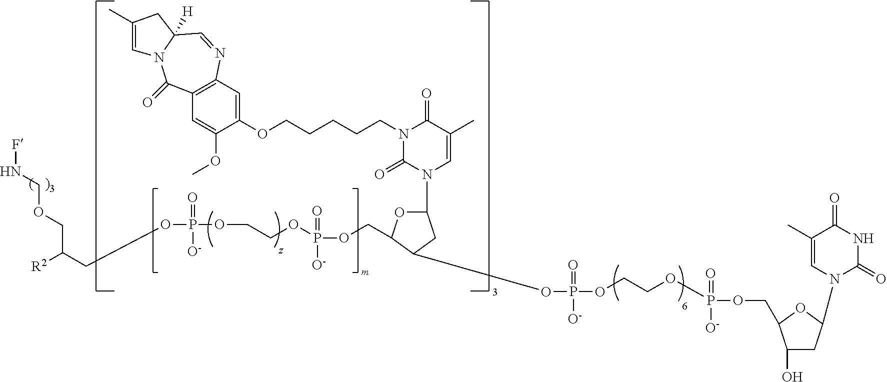 |
| | 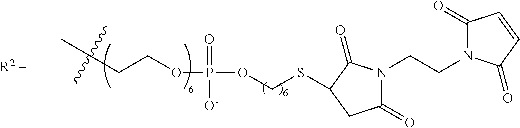 |
| I-30 | 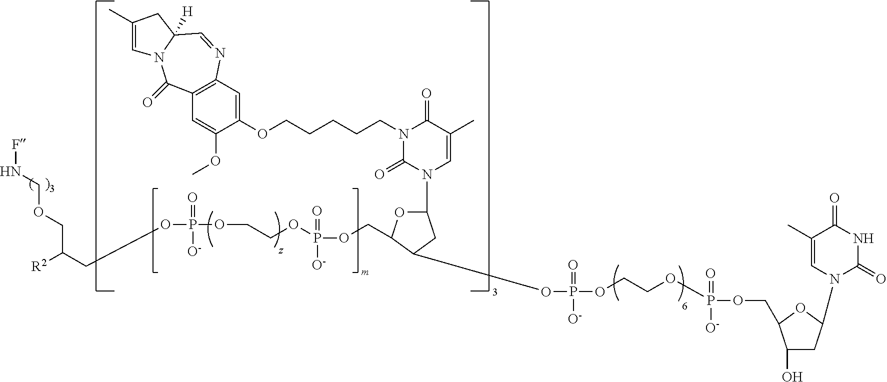 |
| | 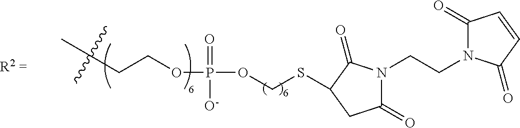 |
| I-31 | 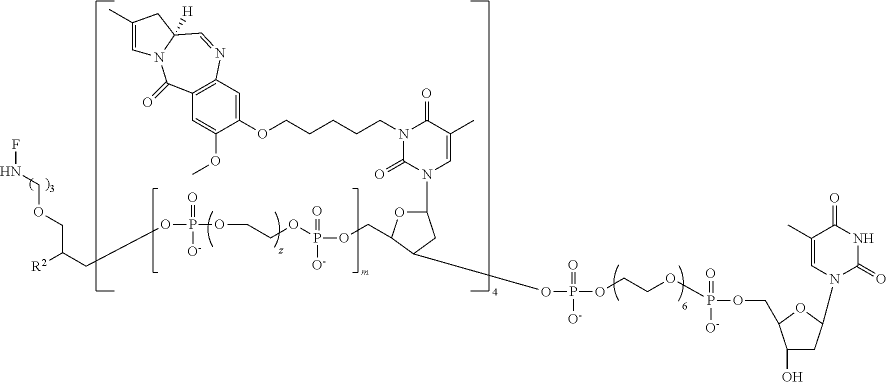 |
| | 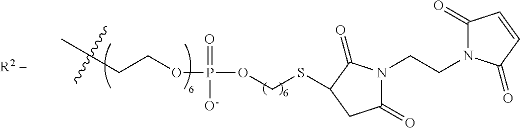 |
| I-32 | 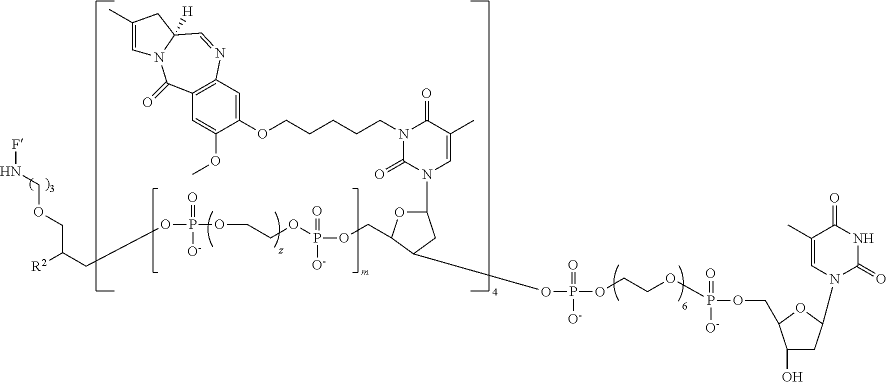 |
| | 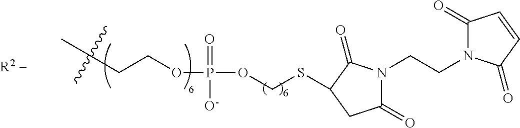 |
| I-33 | 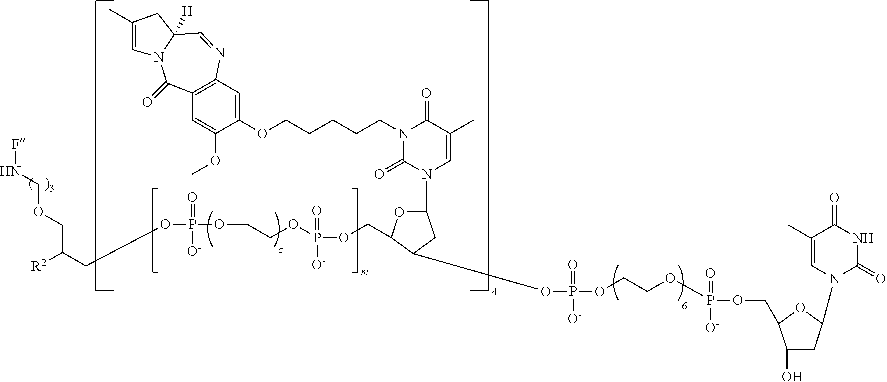 |
| | 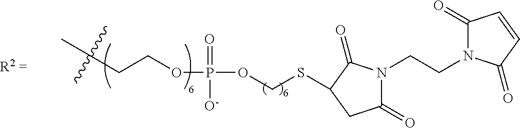 |
| I-34 | 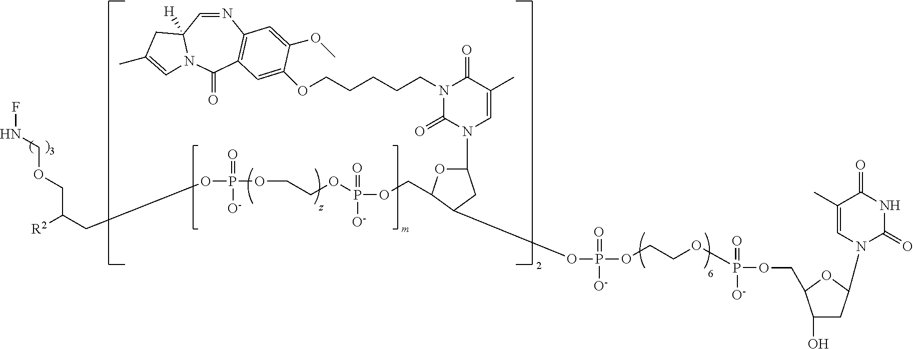 |
| | 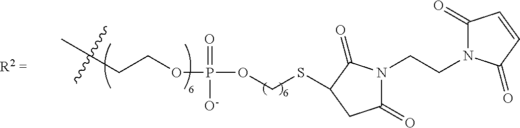 |
| I-35 | 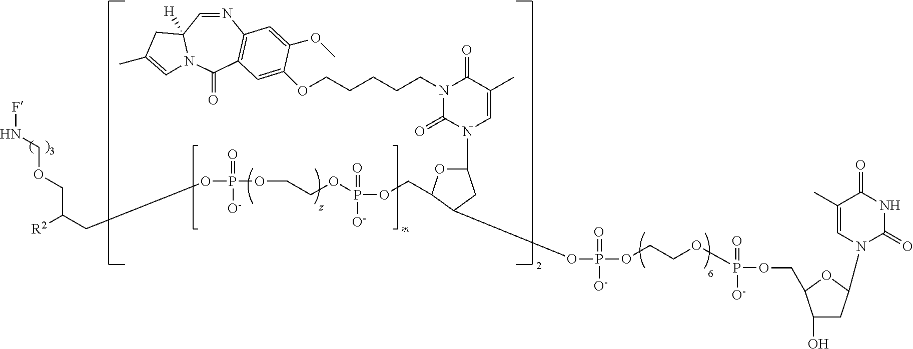 |
| | 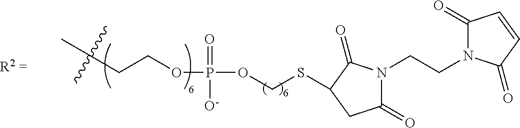 |
| I-36 | 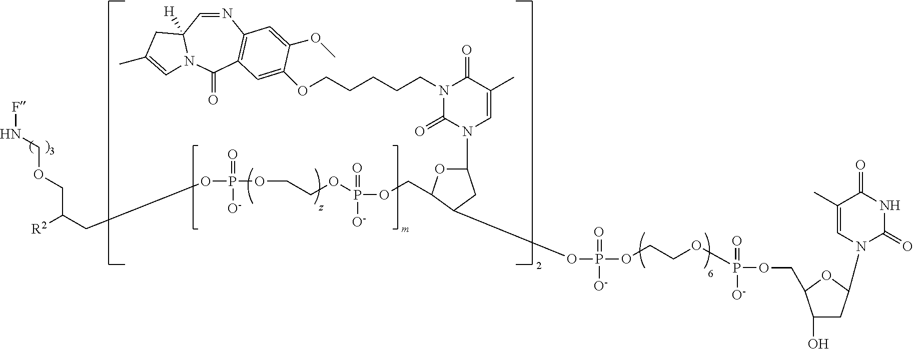 |
| | 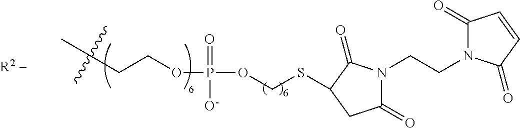 |
| I-37 | 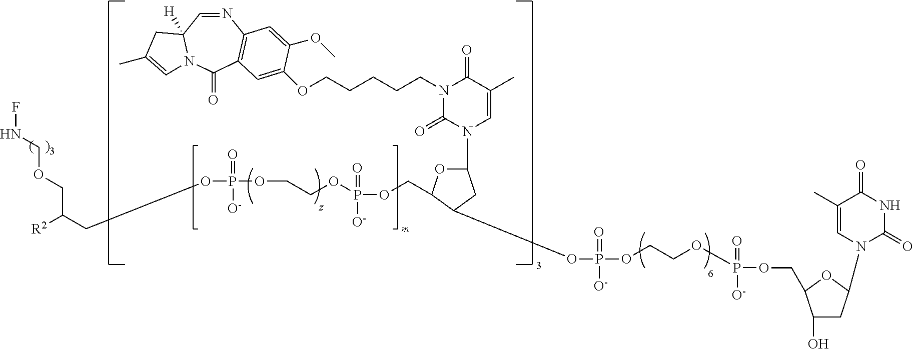 |
| | 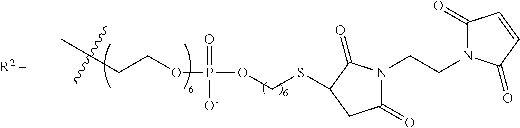 |
| I-38 | 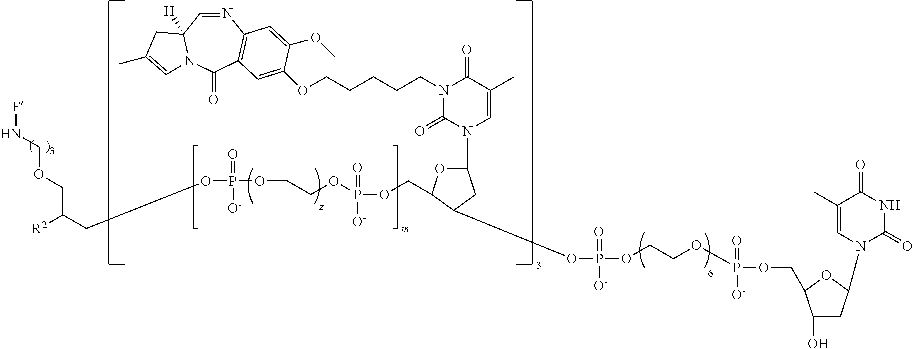 |
| | 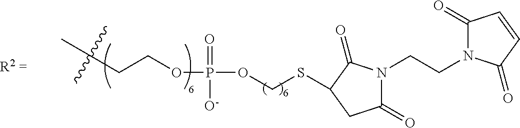 |
| I-39 | 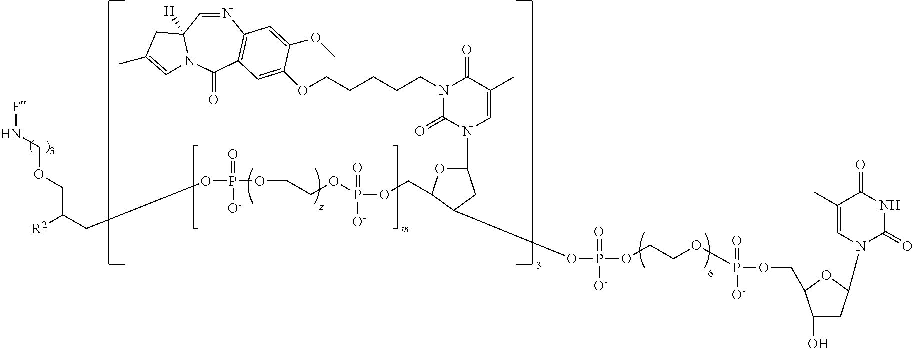 |
| | 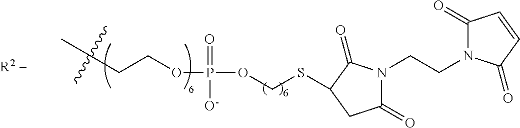 |
| I-40 | 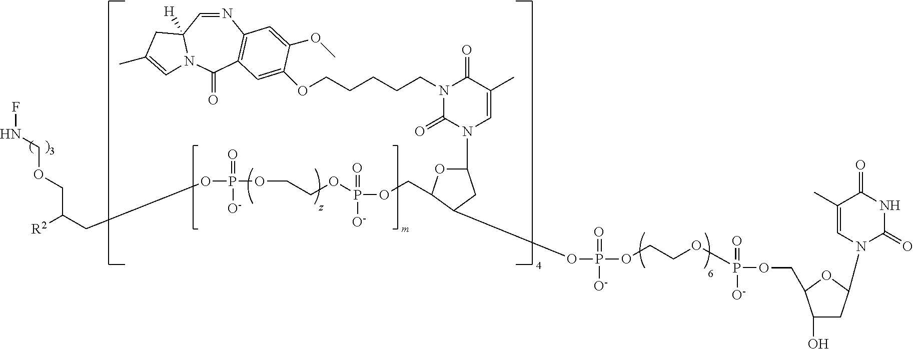 |
| | 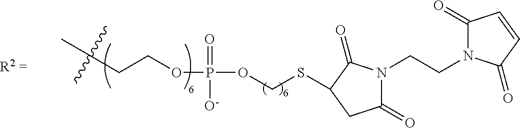 |
| I-41 | 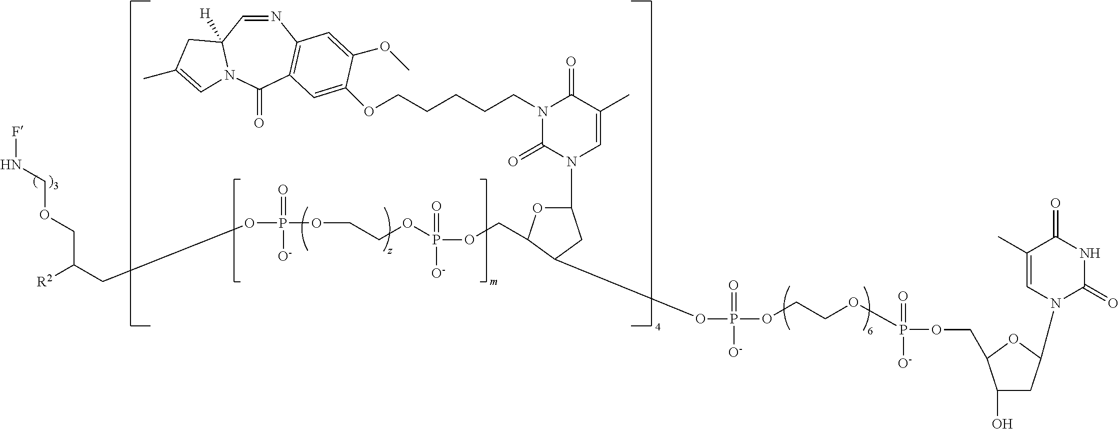 |
| | 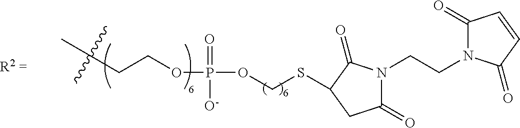 |
| I-42 | 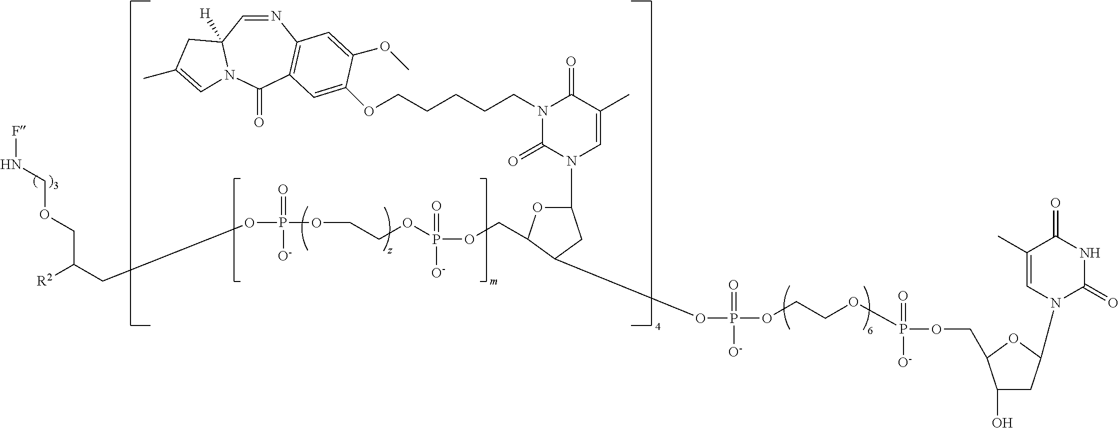 |
| | 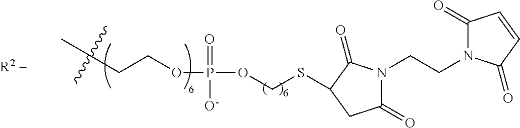 |

TABLE 3B
Exemplary Compounds of Structure (I)

| No. | Structure |
|---|---|
| I-43 | 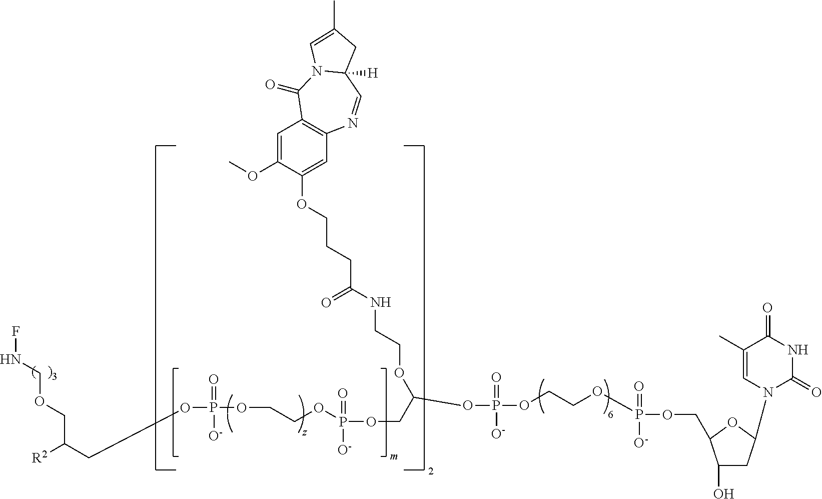 |
| | 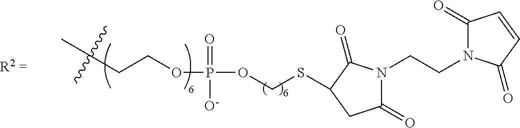 |
| I-44 | 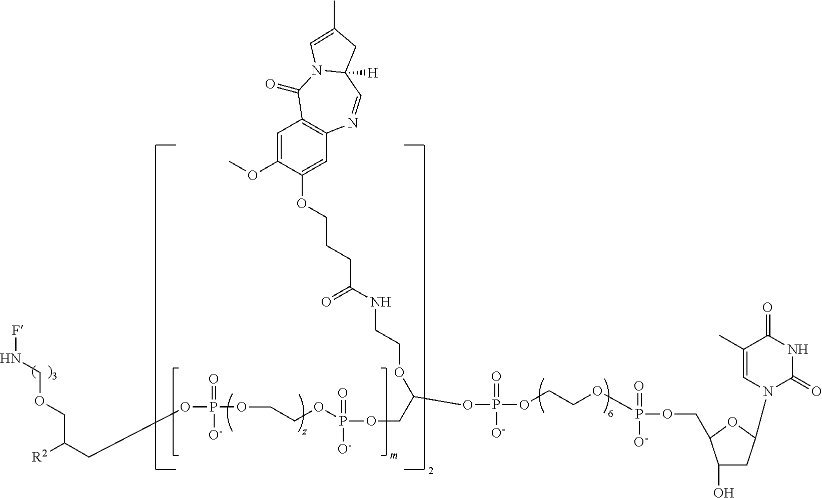 |
| | 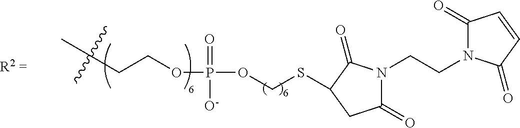 |
| I-45 | 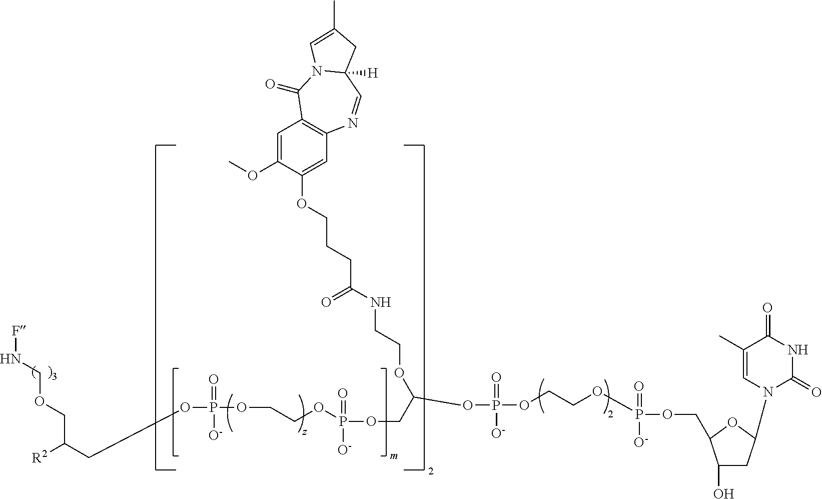 |
| | 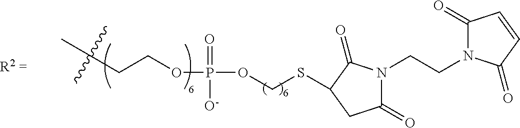 |
| I-46 | 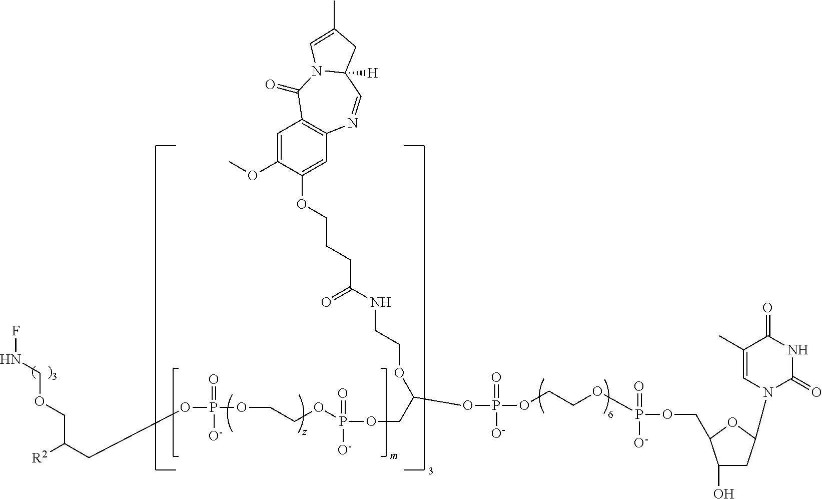 |
| | 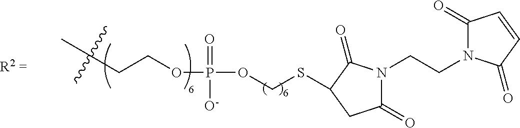 |
| I-47 | 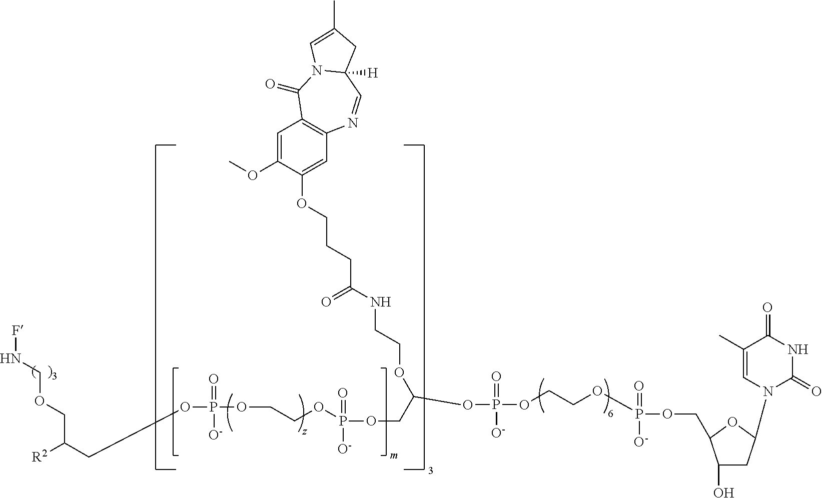 |
| | 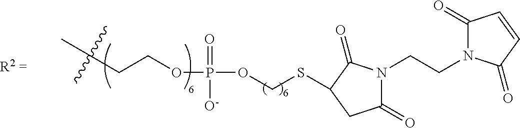 |
| I-48 | 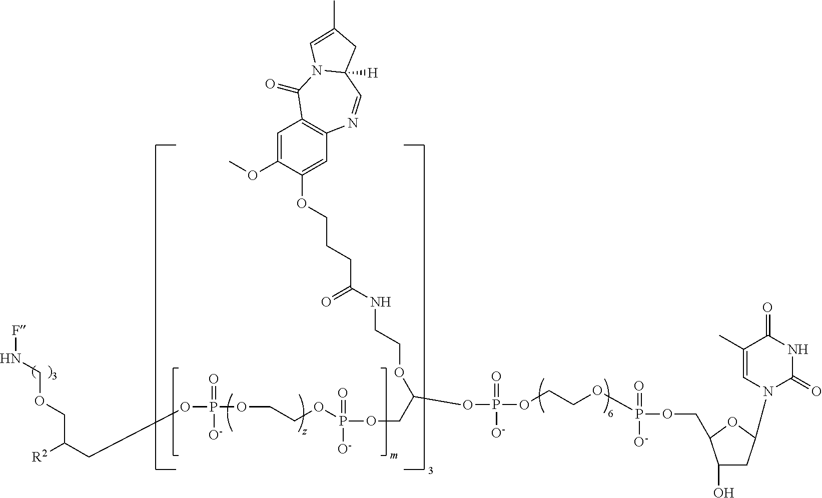 |
| | 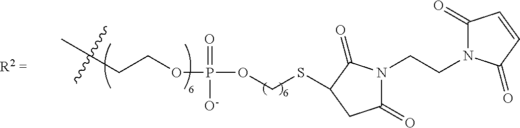 |
| I-49 | 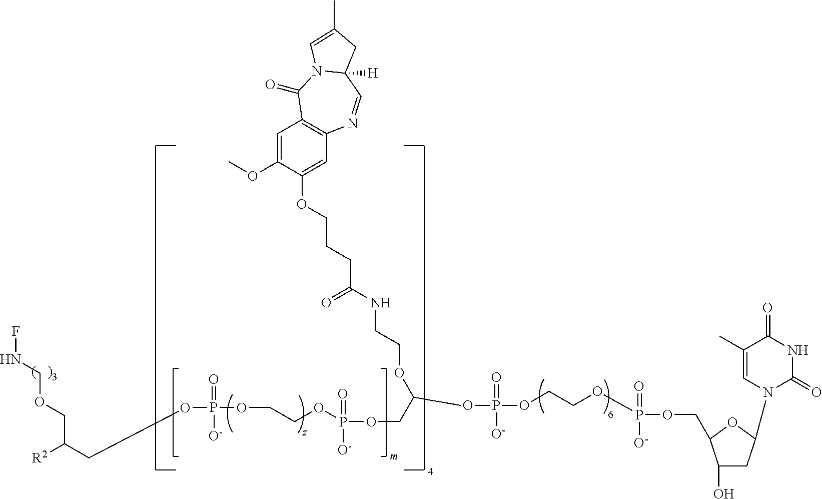 |
| | 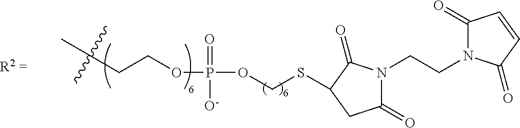 |
| I-50 | 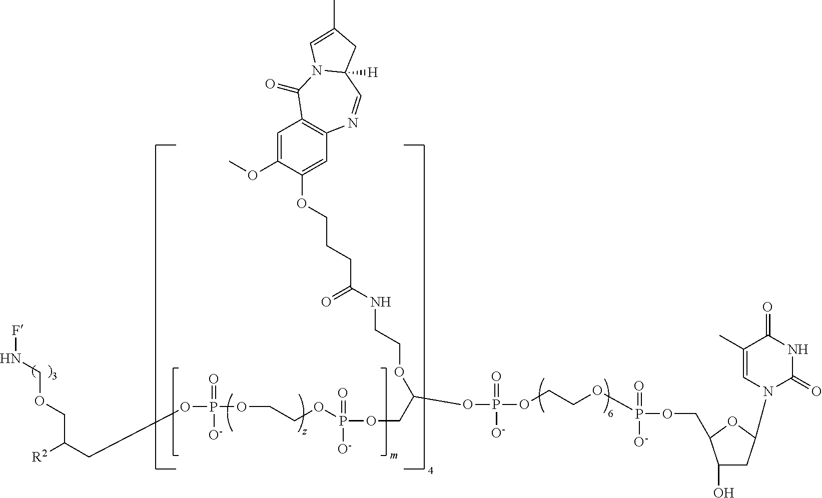 |
| | 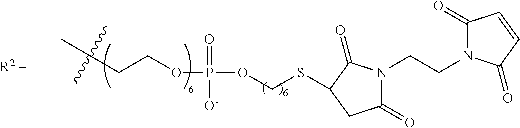 |
| I-51 | 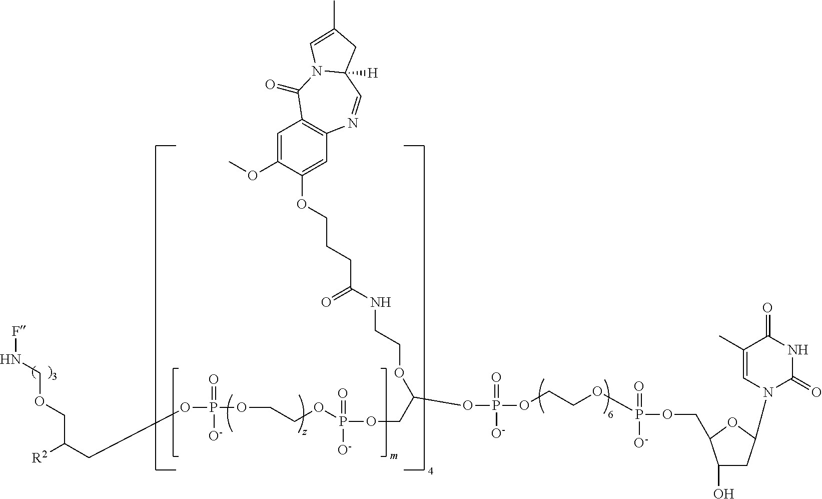 |
| | 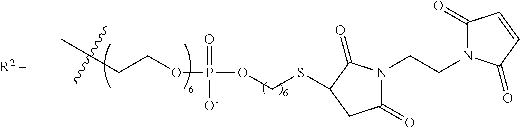 |
| I-52 | 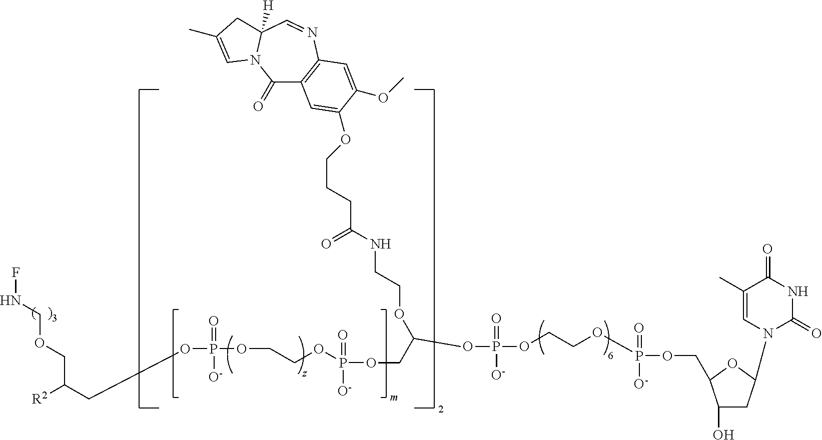 |
| | 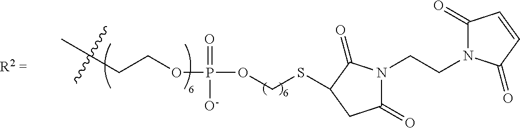 |
| I-53 | 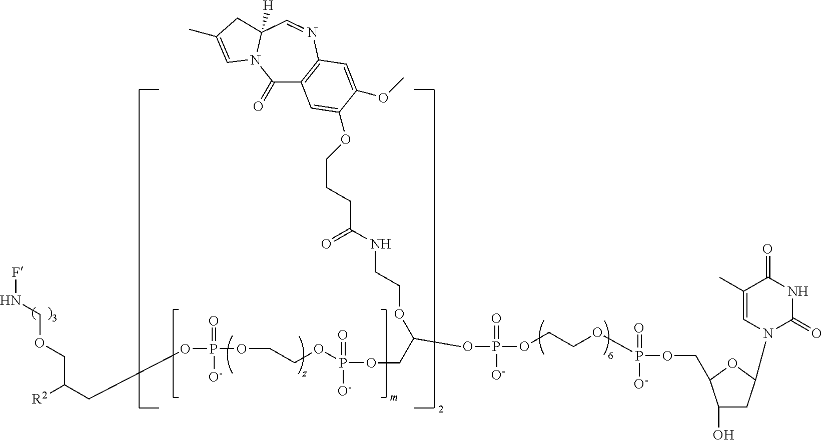 |
| | 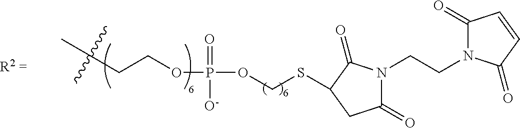 |
| I-54 | 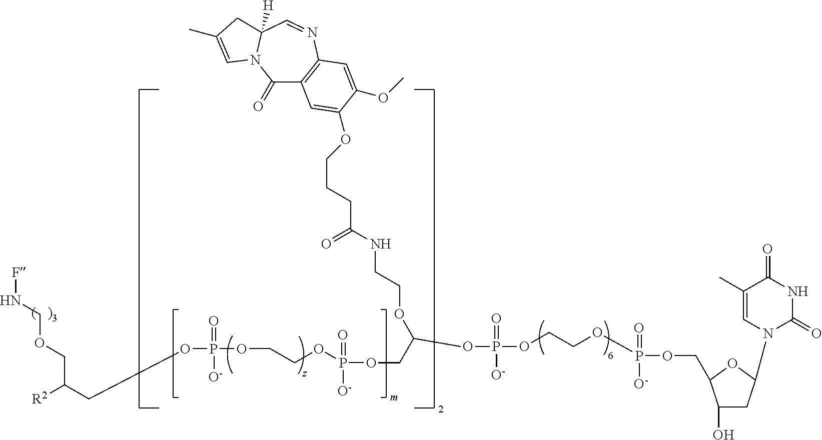 |
| | 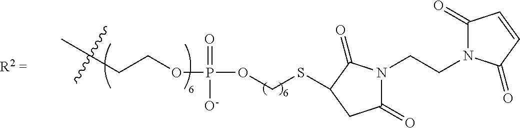 |
| I-55 | 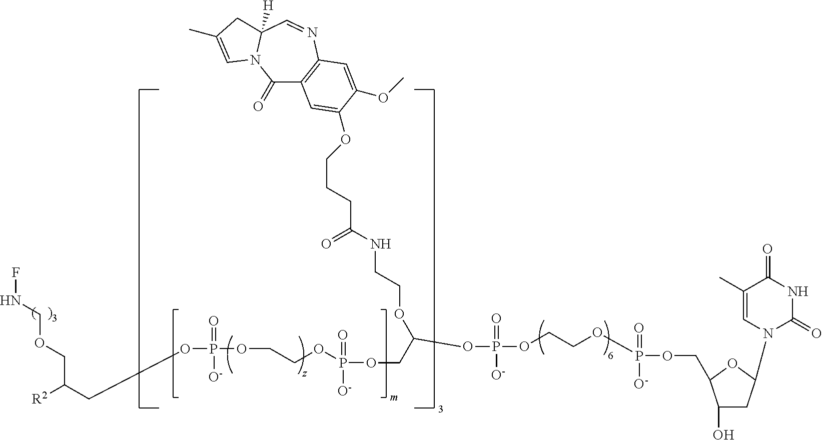 |
| | 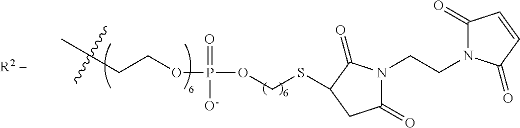 |
| I-56 | 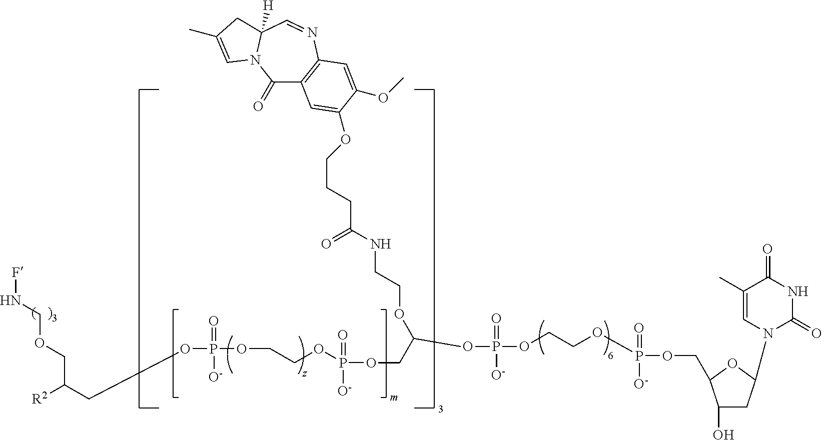 |
| | 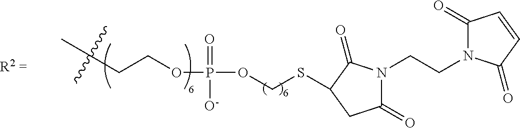 |
| I-57 | 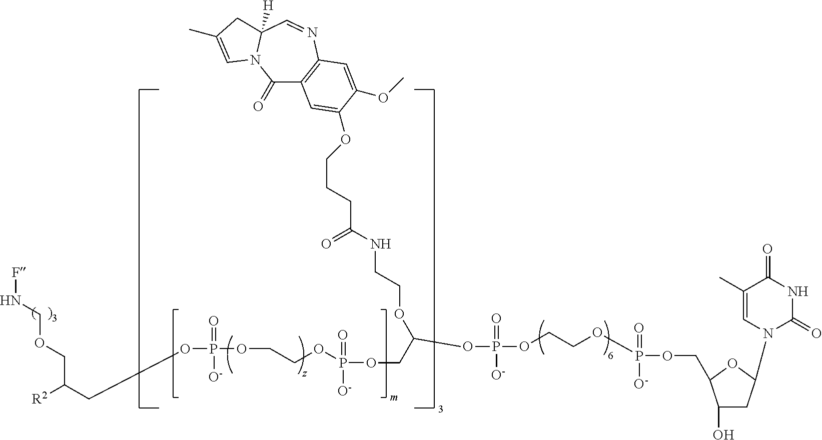 |
| | 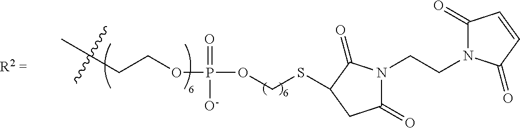 |
| I-58 | 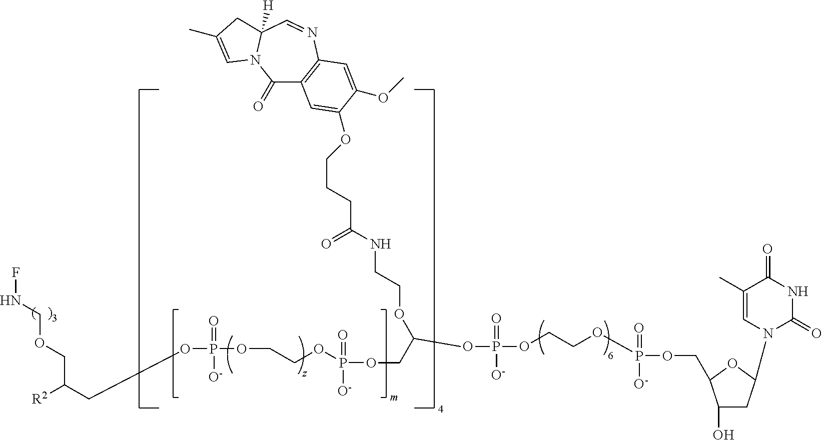 |
| | 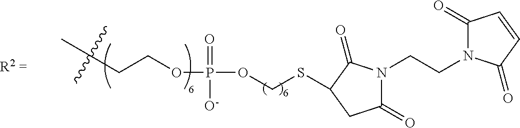 |
| I-59 | 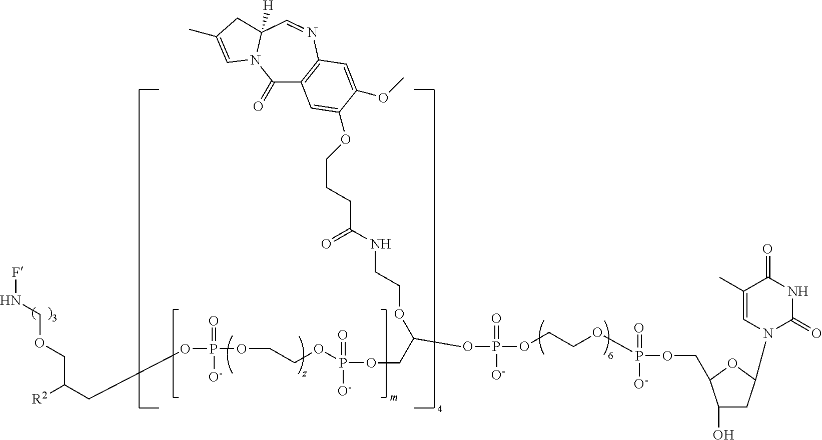 |
| | 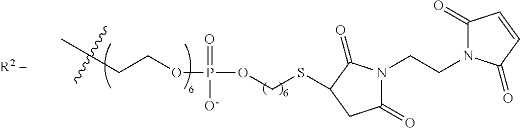 |
| I-60 | 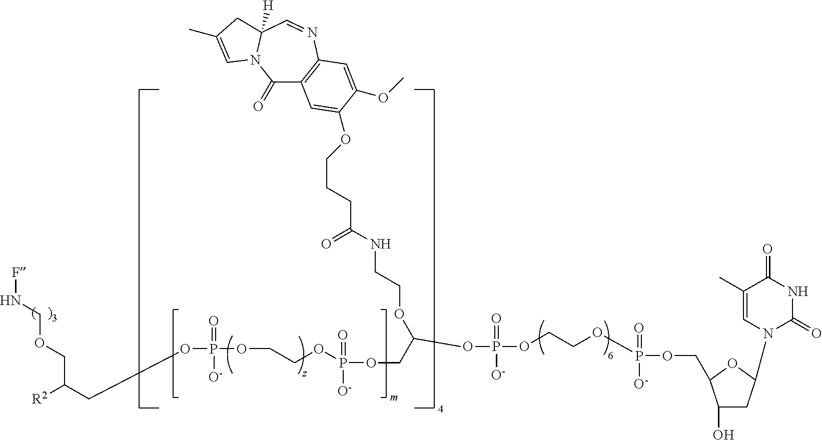 |
| | 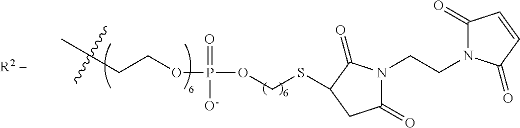 |

As used in Table 3A-3B and throughout the application M has the definitions provided for compounds of structure (I) unless otherwise indicated. In some embodiments, M is F, F′, or F″ refer to a fluorescein moiety having the following structures, respectively:

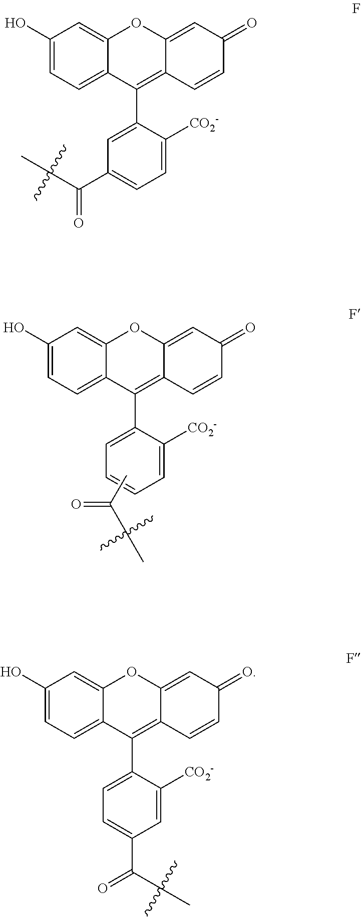

Interaction with DNA

One embodiment provides a compound according any one of the embodiments disclosed herein (e.g., a compound of Structure (I), (Ia), (Ib), (Ic), (Id), or (Ie)) and a pharmaceutically acceptable carrier. In some embodiments, the compounds disclosed in the present disclosure include an electrophilic imine moiety on an alkylating agent such as a PBD moiety (M¹ or M²) at N₁₀—C₁₁ position which can form a covalent bond between C₁₁ carbon and C₂NH₂ group of a guanine base as shown below.

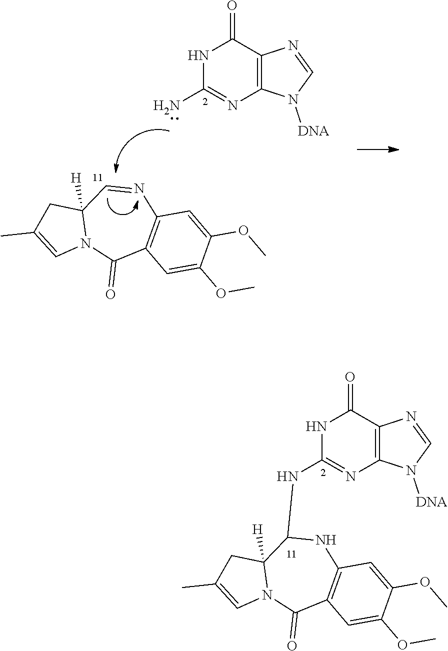

As a result, the PBD moiety of the compounds in the present disclosure can alkylate a guanine base of DNA and form interstrand and intrastrand DNA cross links. The compound having two PBD moieties can interact with DNA to form the following adducts:

### Interstrand Cross-Link

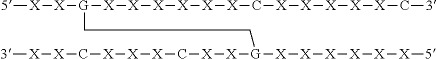

### Intrastrand Cross-Link

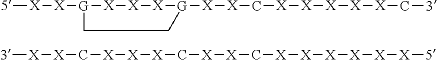

In some embodiments, a compound disclosed herein has two or more PBD moieties as the biological active moieties. For example, the compounds disclosed in the present disclosure may include four PBD moieties. In this regard, the compound can form both interstrand and intrastrand cross link with DNA as shown below:

### Interstrand+Intrastrand Cross-Link

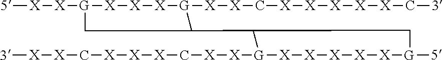

A formation of both interstrand and intrastrand cross links is not possible with a dimer of alkylating agents such as PBD dimers because there are only two alkylation sites in the PBD dimers or compounds having two PBD moieties. A combination of both interstrand and intrastrand cross-links with the compounds disclosed in the present disclosure constitutes an absolute block to DNA strand separation, thus interrupting essential DNA metabolic processes such as replication and transcription. This leads to a stop of dividing cells and eventually cell death. In this regard, compounds disclosed herein which have at least three PBD moieties attached to the polymer backbone are very effective ADC. Further, as the alkylating agents such as the PBD moieties on the polymer backbone of the compounds disclosed herein form interstrand, intrastrand, or both interstrand and intrastrand cross-links with a guanine base (G) of DNA, a triplex formation may be minimized. In some embodiments, the number of PBD moieties attached on the polymer backbone can be controlled in such a way that an efficacy of the compound is maximized depending on how many interstrand and/or intrastrand cross-links are preferred toward treating some solid tumors. As described above, spacing between alkylating agents can be controlled to place an imine moiety of the alkylating agents close to a guanine base of DNA depending on how many other bases are present between two guanines in a DNA strand.

### Pharmaceutical Compositions

One embodiment provides a composition comprising the compound according to any one of the embodiments disclosed herein (e.g., a compound of Structure (I)) and a pharmaceutically acceptable carrier.

Other embodiments are directed to pharmaceutical compositions. The pharmaceutical composition comprises any one (or more) of the compounds of Structure (I) and a pharmaceutically acceptable carrier. In some embodiments, the pharmaceutical composition is formulated for oral administration. In other embodiments, the pharmaceutical composition is formulated for injection. In still more embodiments, the pharmaceutical compositions comprise a compound of Structure (I) and an additional therapeutic agent (e.g., anticancer agent). Non-limiting examples of such therapeutic agents are described herein below.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound of Structure (I) is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound of Structure (I) is provided in the form of a rapid release formulation, in the form of an extended release formulation, or in the form of an intermediate release formulation. In yet other embodiments, the compound of Structure (I) is administered topically.

The compounds of Structure (I) are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that are used in some embodiments. An exemplary dosage is 10 to 30 mg per day. The exact dosage will depend upon the route of administration, the form in which the compound is administered, the subject to be treated, the body weight of the subject to be treated, and the preference and experience of the attending physician.

In some embodiments, a compound of Structure (I) is administered in a single dose. Typically, such administration will be by injection, e.g., intravenous injection, in order to introduce the agent quickly. However, other routes are used as appropriate. A single dose of a compound of Structure (I) may also be used for treatment of an acute condition.

In some embodiments, a compound of Structure (I) is administered in multiple doses. In some embodiments, dosing is about once, twice, three times, four times, five times, six times, or more than six times per day. In other embodiments, dosing is about once a month, once every two weeks, once a week, or once every other day. In another embodiment a compound of Structure (I) and another agent are administered together about once per day to about 6 times per day. In another embodiment the administration of a compound of Structure (I) and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of Structure (I) may continue as long as necessary. In some embodiments, a compound of Structure (I) is administered for more than 1, 2, 3, 4, 5, 6, 7, 14, or 28 days. In some embodiments, a compound of Structure (I) is administered for less than 28, 14, 7, 6, 5, 4, 3, 2, or 1 day. In some embodiments, a compound of Structure (I) is administered chronically on an ongoing basis, e.g., for the treatment of chronic effects.

In some embodiments, the compounds of Structure (I) are administered in dosages. It is known in the art that due to inter-subject variability in compound pharmacokinetics, individualization of dosing regimen is necessary for optimal therapy. Dosing for a compound of the disclosure may be found by routine experimentation in light of the instant disclosure.

In some embodiments, the compounds of Structure (I) are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999).

Provided herein are pharmaceutical compositions comprising a compound of Structure (I) and a pharmaceutically acceptable diluent(s), excipient(s), or carrier(s). In certain embodiments, the compounds described are administered as pharmaceutical compositions in which compounds of Structure (I) are mixed with other active ingredients, as in combination therapy. Encompassed herein are all combinations of actives set forth in the combination therapies section below and throughout this disclosure. In specific embodiments, the pharmaceutical compositions include one or more compounds of Structure (I).

A pharmaceutical composition, as used herein, refers to a mixture of a compound of Structure (I) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, practicing the methods of treatment or use provided herein, therapeutically effective amounts of compounds of Structure (I) provided herein are administered in a pharmaceutical composition to a mammal having a disease, disorder or medical condition to be treated. In specific embodiments, the mammal is a human. In certain embodiments, therapeutically effective amounts vary depending on the severity of the disease, the age and relative health of the subject, the potency of the compound used and other factors. The compounds of Structure (I) are used singly or in combination with one or more therapeutic agents as components of mixtures.

In one embodiment, one or more compounds of Structure (I) is formulated in an aqueous solution. In specific embodiments, the aqueous solution is selected from, by way of example only, a physiologically compatible buffer, such as Hank's solution, Ringer's solution, or physiological saline buffer. In other embodiments, one or more compound of Structure (I) is/are formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the compounds described herein are formulated for buccal or sublingual administration. Formulations suitable for buccal or sublingual administration include, by way of example only, tablets, lozenges, or gels. In still other embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of the active compounds (e.g., compounds of Structure (I)) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

In still other embodiments, the compounds of Structure (I) are administered topically. The compounds described herein are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, the compounds of Structure (I) are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of the compounds of Structure (I) is accomplished by means of iontophoretic patches and the like. In certain embodiments, transdermal patches provide controlled delivery of the compounds of Structure (I). In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In other embodiments, the compounds of Structure (I) are formulated for administration by inhalation. Various forms suitable for administration by inhalation include, but are not limited to, aerosols, mists or powders. Pharmaceutical compositions of any of compound of Structure (I) are conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator is formulated containing a powder mix of the compound and a suitable powder base such as lactose or starch.

In still other embodiments, the compounds of Structure (I) are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with melted cocoa butter.

In certain embodiments, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are optionally used as suitable. Pharmaceutical compositions comprising a compound of Structure (I) are manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and at least one compound of Structure (I), described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical composition comprising at least one compound of Structure (I) illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, useful aqueous suspensions contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful pharmaceutical compositions also, optionally, include solubilizing agents to aid in the solubility of a compound of Structure (I). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, useful pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other useful pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In some embodiments, the concentration of one or more compounds provided in the pharmaceutical compositions is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of one or more compounds is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more compounds is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of one or more compounds is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of one or more compounds ranges from 0.0001 to 10 g, 0.0005 to 9 g, 0.001 to 8 g, 0.005 to 7 g, 0.01 to 6 g, 0.05 to 5 g, 0.1 to 4 g, 0.5 to 4 g, or 1 to 3 g.

### Method of Treatment

Certain compounds of the present disclosure are useful for treating disease (i.e., compounds of Structure (I)). Those compounds disclosed herein offer a targeted approach to drug delivery strategies. Accordingly, one embodiment provides a method of treating a disease (or the symptoms thereof) comprising administering to a mammal (e.g., a human) in need thereof a therapeutically effective amount of a compound of Structure (I).

For example, in certain embodiments the disclosure provides a method of treating solid tumors, multiple myeloma, gliomas, clear cell renal cell carcinoma, prostate cancer, ovarian cancer, non-small cell lung cancer, GI malignancies, acute lymphoblastic leukemia, acute myelogenous leukemia, renal cell carcinoma, colorectal carcinoma, epithelial cancers, pancreatic and gastric cancers, renal cell carcinoma, non-Hodgkin's lymphoma, metastatic renal cell carcinoma, malignant mesothelioma, pancreatic, ovarian, and/or lung adenocarcinoma, B-cell malignancies, breast cancer, melanoma, recurrent multiple myeloma, small cell lung cancer, CD22-positive B cell malignancies, Hodgkin's lymphoma/anaplastic large cell lymphoma, or HER2-positive breast cancer.

In some of the foregoing embodiments, the disease is cancer. For example, in certain embodiments, the cancer is breast cancer, non-Hodgkin's lymphoma, acute myeloid leukemia, multiple myeloma, gastric cancer, renal cell carcinoma, solid tumors, ovarian cancer, prostate cancer, colorectal cancer, pancreatic cancer, small cell lung cancer, diffuse large B-cell lymphoma, a neoplasm, urothelial cancer, ALL, CLL, glioblastoma, Hodgkin's lymphoma, lymphoma, mesothelioma, non-small cell lung cancer, recurrent head and neck cancer, or a combination thereof.

Certain embodiments also relate to a method of treating a hyperproliferative disorder in a mammal (e.g., a human) that comprises administering to said mammal a therapeutically effective amount of a compound of Structure (I), or a pharmaceutically acceptable salt, ester, prodrug, solvate, hydrate or derivative thereof. In some embodiments, said method relates to the treatment of cancer such as acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, Burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin's lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin's lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some embodiments, said method relates to the treatment of a non-cancerous hyperproliferative disorder such as benign hyperplasia of the skin (e.g., psoriasis), restenosis, or prostate (e.g., benign prostatic hypertrophy (BPH)).

Certain particular embodiments provide methods for treatment of lung cancers, the methods comprise administering an effective amount of any of the above described compounds of Structure (I) (or a pharmaceutical composition comprising the same) to a subject in need thereof. In certain embodiments the lung cancer is a non-small cell lung carcinoma (NSCLC), for example adenocarcinoma, squamous-cell lung carcinoma or large-cell lung carcinoma. In other embodiments, the lung cancer is a small cell lung carcinoma. Other lung cancers treatable with the disclosed compounds include, but are not limited to, glandular tumors, carcinoid tumors and undifferentiated carcinomas.

Accordingly, in some embodiments of Structure (I) A is an antibody or a cell surface receptor antagonist. For example, epidermal growth factor receptor (EGFR) inhibitor, a hepatocyte growth factor receptor (HGFR) inhibitor, an insulin-like growth factor receptor (IGFR) inhibitor, a folate, or a MET inhibitor. What about the specific antibodies trastuzumab, etc.

In even more embodiments, the method further comprises inducing apoptosis.

In some embodiments, the method of treatment comprises treating a tumor having tumor cells with tumor cell receptors. In some embodiments, the tumor cells have receptors ranging from 1,000 to 100,000, from 1,000 to 50,000, from 1,000 to 25,000 receptors, 1,000 to 10,000 receptors per cell. For example, in some embodiments the tumor cells have about 1,000, about 10,000, or less than 100,000 receptors per cell.

Further therapeutic agents that can be combined with a compound of the disclosure are found in Goodman and Gilman's “The Pharmacological Basis of Therapeutics” Tenth Edition edited by Hardman, Limbird and Gilman or the Physician's Desk Reference, both of which are incorporated herein by reference in their entirety.

The compounds of Structure (I) described herein can be used in combination with the agents disclosed herein or other suitable agents, depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the disclosure and any of the agents described above can be simultaneously administered, wherein both the agents are present in separate formulations. In another alternative, a compound of the present disclosure can be administered just followed by and any of the agents described above, or vice versa. In some embodiments of the separate administration protocol, a compound of the disclosure and any of the agents described above are administered a few minutes apart, or a few hours apart, or a few days apart.

In some embodiments, the method further comprises administering an additional therapeutic agent selected from the group consisting of an antineoplastic agent, an enediyne antitumor antibiotic, a maytansinoid, a topoisomerase inhibitor, a kinase inhibitor, an anthracycline, and EGFR inhibitor, an alkylating agent and combinations thereof.

In some more specific embodiments, the method further comprises administering an additional therapeutic agent selected from the group consisting of an antineoplastic agent, an enediyne antitumor antibiotic, a maytansinoid, a topoisomerase inhibitor, a kinase inhibitor, an anthracycline, and EGFR inhibitor, an alkylating agent and combinations thereof.

In certain embodiments, the additional therapeutic agent comprises auristatin F, monomethyl auristatin F, monomethyl auristatin E, paciltaxol, SN-38, calicheamicin, anthramycin, abbeymycin, chicamycin, DC-81, mazethramycin, neothramycin A, neothramycin B, porothramycin prothracarcin, sibanomicin, sibiromycin, tomamycin, mertansine, emtansine, irinotecan, camptothecin, topotecan, silatecan, cositecan, Exatecan, Lurtotecan, gimatecan, Belotecan, and Rubitecan.

In some embodiments, a pharmaceutical composition comprising the compound of any of the forementioned structures, and a pharmaceutically acceptable carrier, diluent, or excipient. In some embodiments, a method of treating a disease or disorder, comprising administering a therapeutically effective amount of a compound of any of the forementioned structures, or the pharmaceutical composition of the same, to a subject in need thereof. In certain embodiments, the disease or disorder is cancer. In some more specific embodiments, the cancer is breast cancer, stomach cancer, lung cancer, ovarian cancer, lymphoma, and bladder cancer.

The examples and preparations provided below further illustrate and exemplify the compounds of the present disclosure and methods of preparing such compounds. It is to be understood that the scope of the present disclosure is not limited in any way by the scope of the following examples and preparations. In the following examples, and throughout the specification and claims, molecules and moieties with a single stereocenter, unless otherwise noted, exist as a racemic mixture. Those molecules and moieties with two or more stereocenters, unless otherwise noted, exist as a racemic mixture of diastereomers. Single enantiomers/diastereomers may be obtained by methods known to those skilled in the art.

As described in detail above, compounds of structures (I), (Ia). (Ib), (Ic), (Id), and (Ie) can be prepared by oligomerization using well known phosphoramidite chemistry. Applicants have discovered intermediate compounds useful for synthesis of compounds of structures (II) and compounds of structure (III). Accordingly, embodiments of the present disclosure provide a compound having one of the following structures (II) or (III):

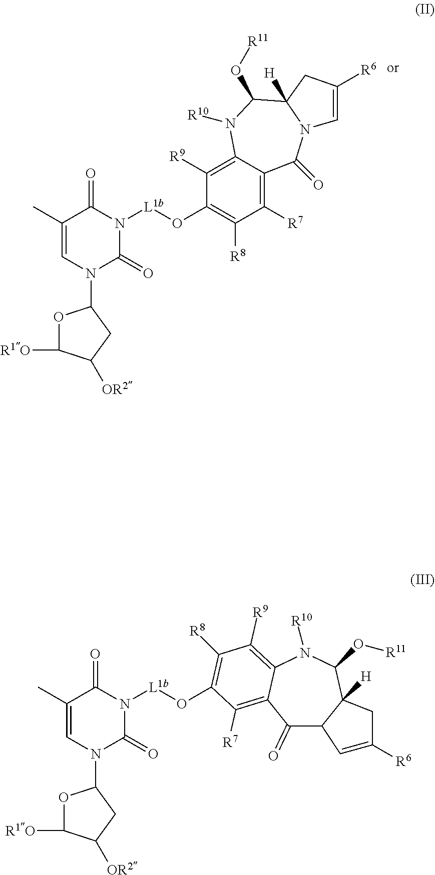

In some embodiments, R¹″ is H, a protecting group, or an activated phosphorus moiety. For example, in some specific embodiments, R¹″ is a dimethoxytrityl group (TMD). The TMD protecting group can be cleaved to afford a hydroxyl group with a fluoride source such as tetra-n-butylammonium fluoride (TBAF).

In some embodiments, R²″ is H or has the following phosphoramidite structure.

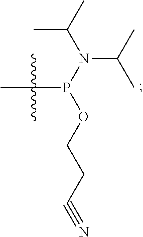 [SMILES: *P(OCCC#N)N(C(C)C)C(C)C]

The above phosphoramidite moiety can be installed by reacting a free hydroxyl group (unprotected) of the structure (II) or (III) with 3-((chloro(diisopropylamino)phosphaneyl)oxy)propanenitrile under a basic condition as described in the following section.

In some embodiments, R⁶ is H, CH═CHCONH2, alkyl, alkoxy, heteroalkyl, heteroalkoxy, alkylether, alkoxyalkylether, cycloalkyl, heterocyclyl, aryl, or heteroaryl. For example, in some specific embodiments, R⁶ is an alkyl group such as a methyl group (—CH₃).

In some embodiments, R⁷, R⁸, and R⁹ are independently H, OH, OR_{f}, SH, SR_{f}, NH₂, NHR_{f}, NR_{f}R_{g}, alkyl, alkoxy, alkylether, or heteroalkyl. For example, in some specific embodiments, R⁷ and R⁹ are independently H. R⁸ is an alkoxy group.

For instance, R⁸ is a methoxy group (—OCH₃).

In some embodiments, R¹⁰ is a nitrogen protecting group or H. For example, in some specific embodiments, R¹⁰ is an allylocycarbonyl (Alloc). The Alloc protecting group can be easily cleaved to afford the corresponding amine with a Pd metal based condition. In some embodiments, other nitrogen protecting groups can be used instead. For instance, the nitrogen protecting groups of R¹⁰ include benzylocycarbonyl (Cbz), 9-fluorenylmethoxycarbonyl (Fmoc), or tert-butyloxycarbonyl (Boc).

In some embodiments, R¹¹ is an oxygen protecting group, alkyl, or H. For example, in some specific embodiments, R¹¹ is tert-butyldimethylsilyl ether (TBS) group. In some embodiments, other silyl ether containing oxygen protecting groups can be used instead. For instance, the oxygen protecting groups of R¹¹ include trimethylsilyl ether (TMS), triethylsilyl ether (TES), tert-butyldiphenylsilyl ether (TBDPS), or triisopropylsilyl ether (TIPS). Silyl ether protecting groups can be cleaved to afford a hydroxyl group with a fluoride source such as tetra-n-butylammonium fluoride (TBAF).

In some embodiments, L^{1b} is an optionally alkylene or an optionally heteroalkylene linker. For example, in some specific embodiments, L^{1b} comprises an alkyl chain with an odd numbered carbon atoms such as C₃, C₅, or C₇. In other embodiments, L^{1b} comprises an alkyl chain with an even numbered carbon atoms such as C₂, C₄, or C₆.

In some embodiments, a compound has one of the following structures (IIa) or (IIIa):

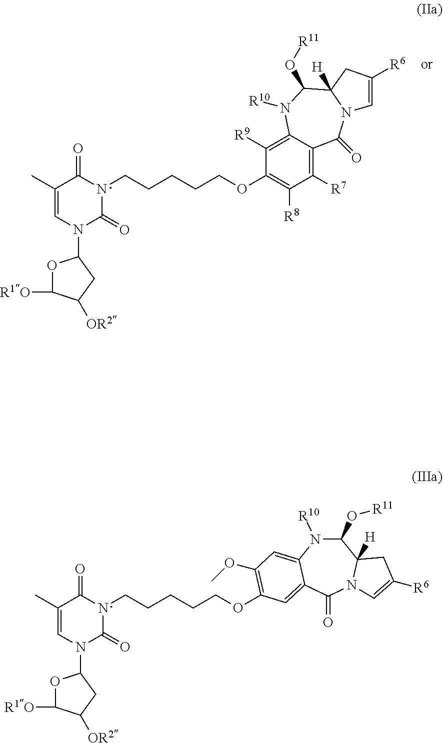

or a stereoisomer,
wherein:
R¹″ is H, a protecting group, or an activated phosphorus moiety;
R²″ is H or has the following structure:

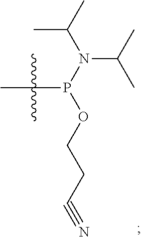 [SMILES: *P(OCCC#N)N(C(C)C)C(C)C]

R⁶ is a methyl;
R¹⁰ is a nitrogen protecting group or H; and
R¹¹ is an oxygen protecting group or H.

In some specific embodiments, a compound (II), (IIa), (III), or (IIIa) is selected from Table 4 or Table 5, respectively.

TABLE 4
Exemplary Compounds of Structure II

| No. | Structure |
|---|---|
| II-1 | 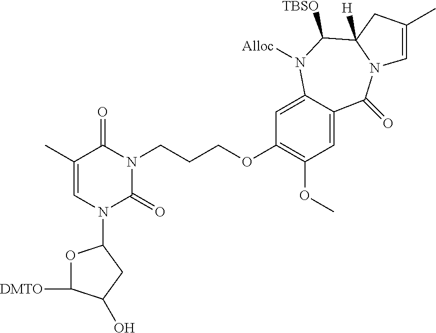 |
| II-2 | 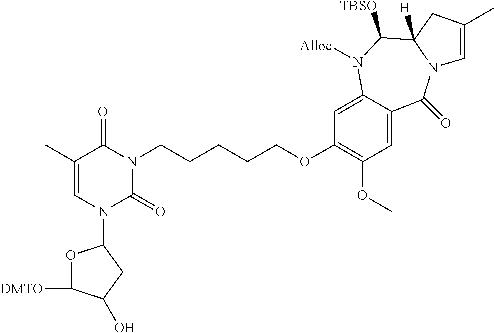 |
| II-3 | 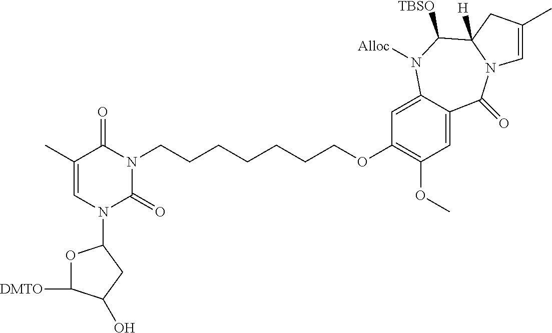 |
| II-4 | 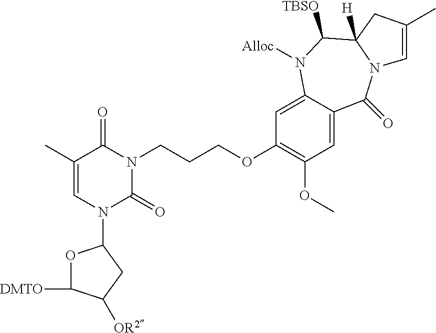 |
| | wherein R^{2″} is |
| | 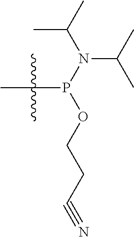 [SMILES: *P(OCCC#N)N(C(C)C)C(C)C] |
| II-5 | 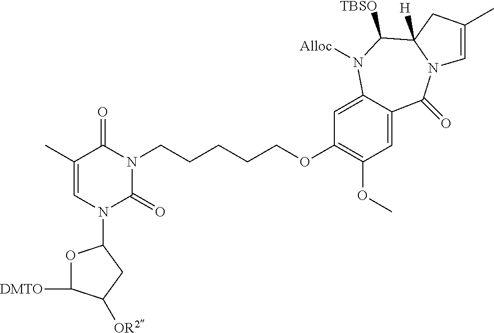 |
| | wherein R^{2″} is |
| | 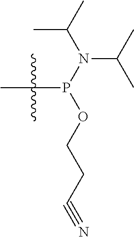 [SMILES: *P(OCCC#N)N(C(C)C)C(C)C] |
| II-6 | 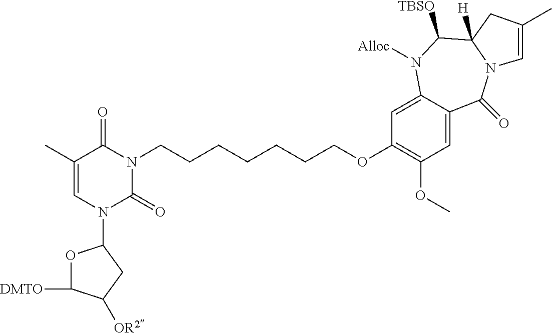 |
| | wherein R^{2″} is |
| | 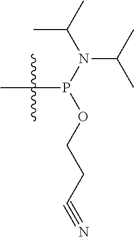 [SMILES: *P(OCCC#N)N(C(C)C)C(C)C] |

TABLE 5
Exemplary Compounds of Structure III

| No. | Structure |
|---|---|
| III-1 | 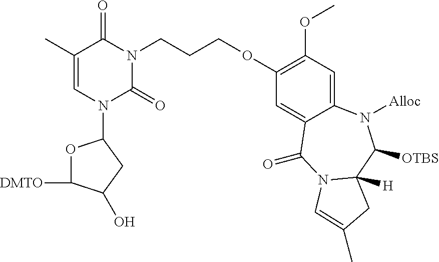 |
| III-2 | 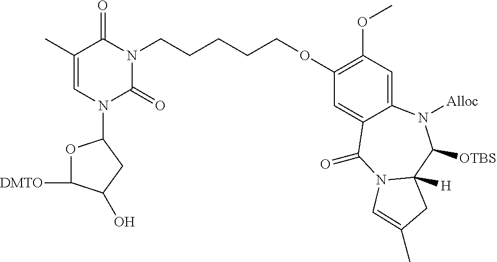 |
| III-3 | 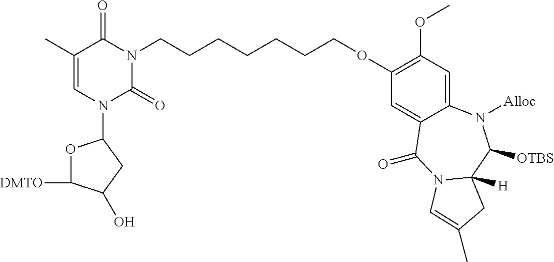 |
| III-4 | 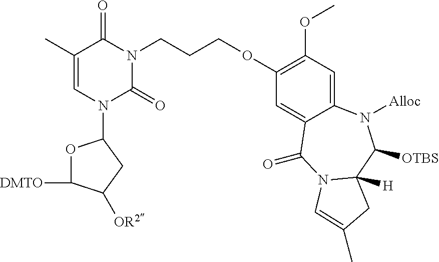 |
| | wherein R^{2″} is |
| | 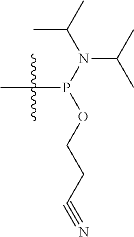 [SMILES: *P(OCCC#N)N(C(C)C)C(C)C] |
| III-5 | 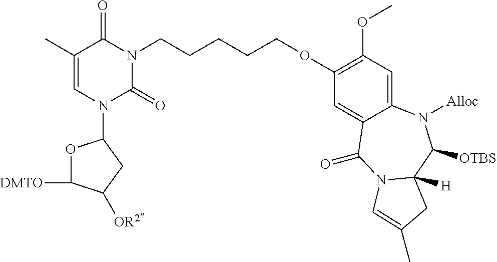 |
| | wherein R^{2″} is |
| | 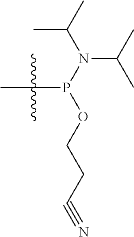 [SMILES: *P(OCCC#N)N(C(C)C)C(C)C] |
| III-6 | 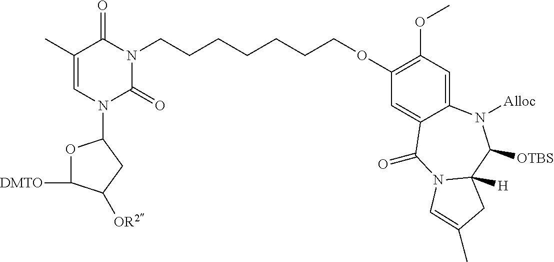 |
| | wherein R^{2″} is |
| | 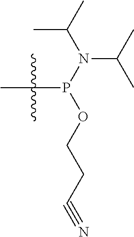 [SMILES: *P(OCCC#N)N(C(C)C)C(C)C] |

TABLE 6
Exemplary Compounds of solketal based Structure

| | |
|---|---|
| IV-1 | 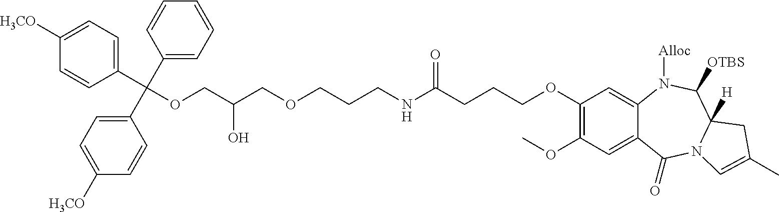 |
| IV-2 | 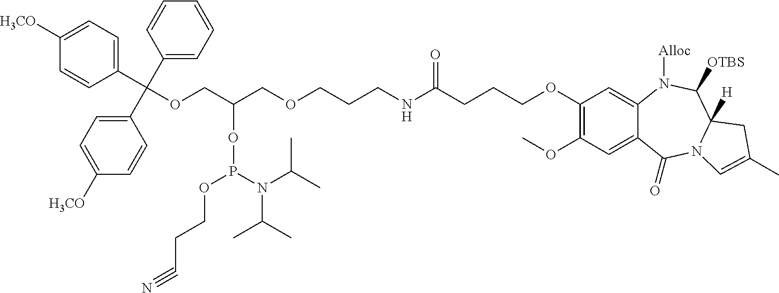 |
| IV-3 | 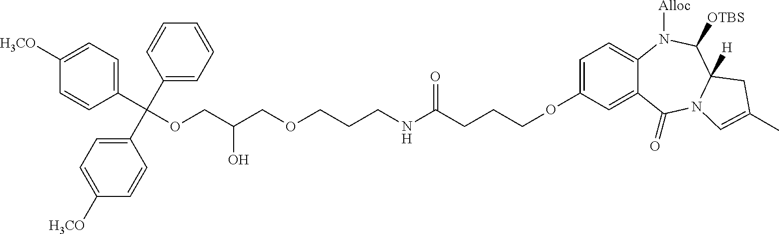 |
| IV-4 | 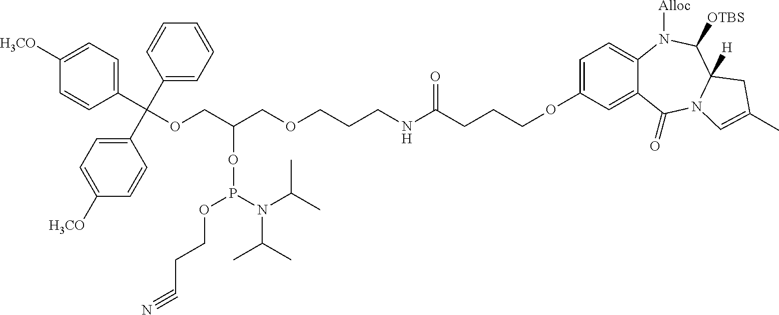 |

The following examples are provided for purposes of illustration, not limitation.

### A Representative DNA Synthesis Cycle

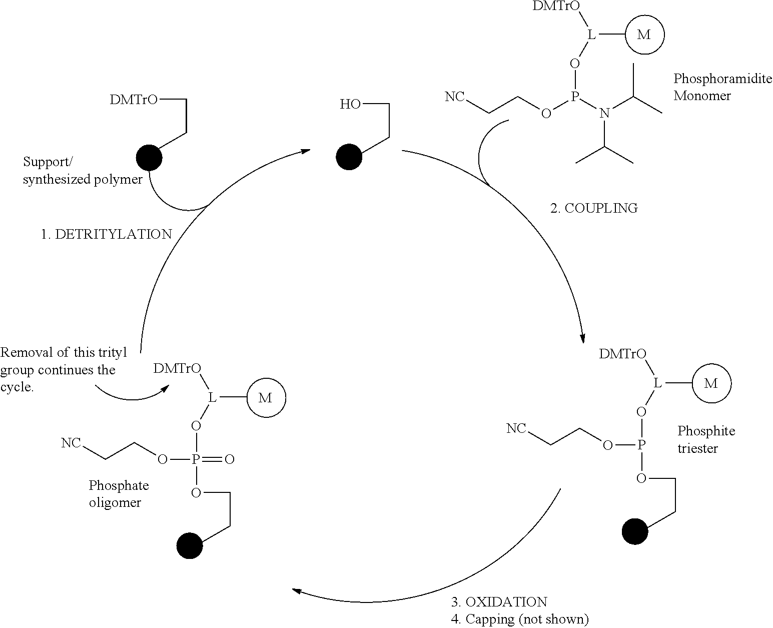

Oligomerization is initiated, typically, through the removal of a protecting group (e.g. a dimethoxytrityl group, DMTr) to reveal a free —OH (hydroxyl) group (Step 1, DETRITYLATION). In a subsequent coupling step, a phosphoramidite monomer is introduced that reacts with the free OH group making a new covalent bond to phosphorus, with concomitant loss of the diisopropyl amine group (Step 2, COUPLING). The resultant, phosphite triester is oxidized (e.g. with 12 and pyridine) to the more stable phosphate ester (Step 3, OXIDATION) and a capping step renders unreactive any remaining free OH groups (Step 4, CAPPING). The new product, phosphate oligomer, contains a DMTr protected OH group that can be deprotected to reinitiate the synthetic cycle so another phosphoramidite monomer can be appended to the oligomer.

Customization occurs at step 2 through the choice of phosphoramidite monomer. The nature of L (i.e., a linker group) and M (i.e., a chemotherapeutic agent) in the scheme above are selected such that a desired compound of Structure (I), (Ia), (Ib), (Ic), (Id), and (Ie) is synthesized. M can be optionally absent to incorporate desired spacing between M moieties. A person of ordinary skill in the art can select multiple monomer types to arrive at compounds of the disclosure containing multiple therapeutic agents and/or other moieties (e.g., fluorophores or chromophores) with concurrent variability in linker groups.

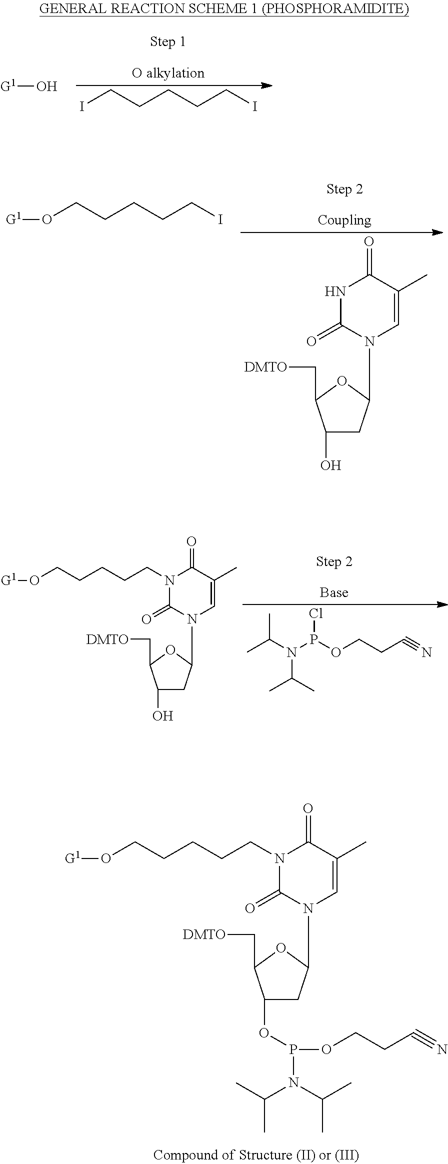

Reaction Scheme I illustrates a method for preparation of phosphoramidite intermediates useful for preparation of compounds of Structure (I), (Ia), (Ib), (Ic), (Id), and (Ie). Referring to Reaction Scheme I, G¹ represents a desired alkylating agent moiety containing a hydroxyl functional group (e.g., an alkylating agent moiety such as pyrrolo benzo diazepine). Step 1 of Reaction Scheme I starts with alkylation on the oxygen of the hydroxyl group with alkyl halide such as 1,5-diiodopentane shown using known reagents under basic conditions (e.g., K₂CO₃ in acetone). The resulting ether is then coupled with TMD protected thymidine to provide the reaction product of Step 2. The resulting adduct is then reacted with 3-((chloro(diisopropylamino)phosphaneyl)oxy)propanenitrile (or other appropriate reagent) to yield a desired compound of Structure (II) or (III) as shown above.

The resultant compound of Structure (II) or (III) can then be used to synthesize a desired compound of Structure (I), (Ia), (Ib), (Ic), (Id), and (Ie) by reaction under well-known (automated) DNA synthesis conditions. In some specific embodiments, the following reaction scheme may be used for the synthesis of a compound III-5 shown in Table 5.

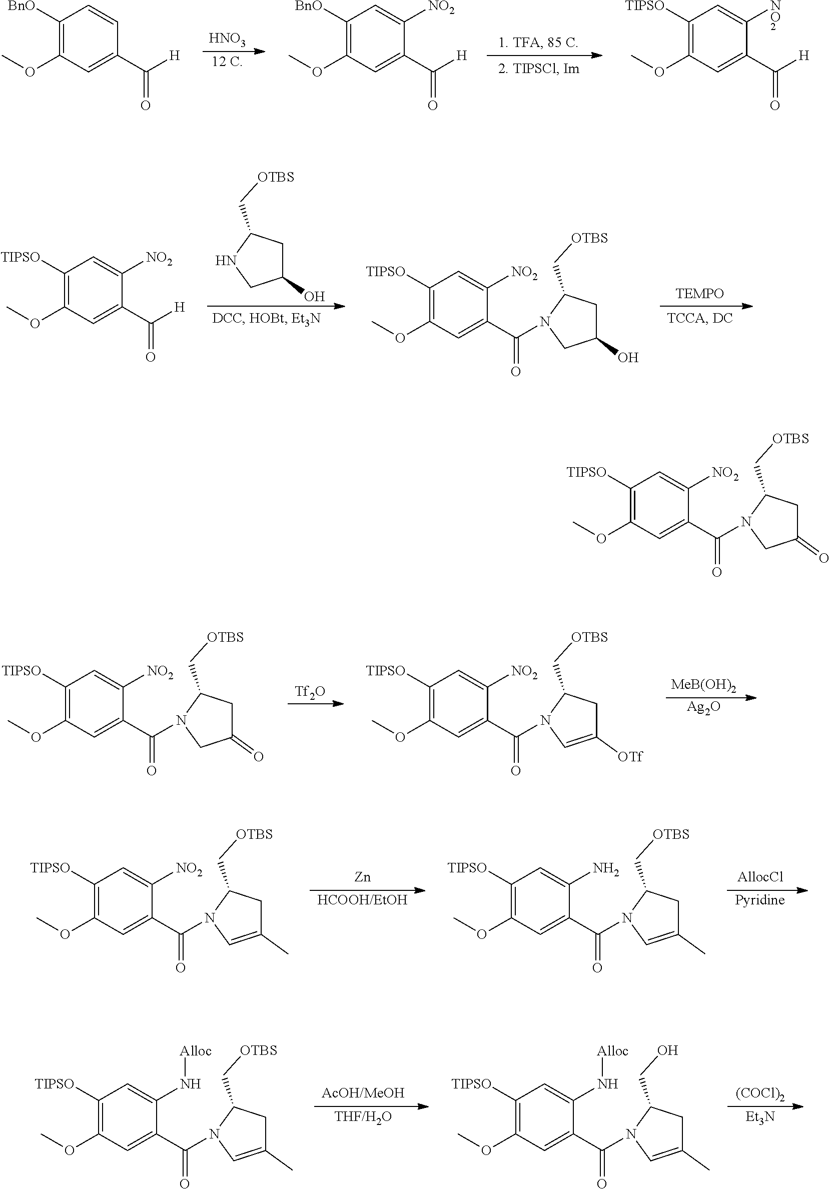

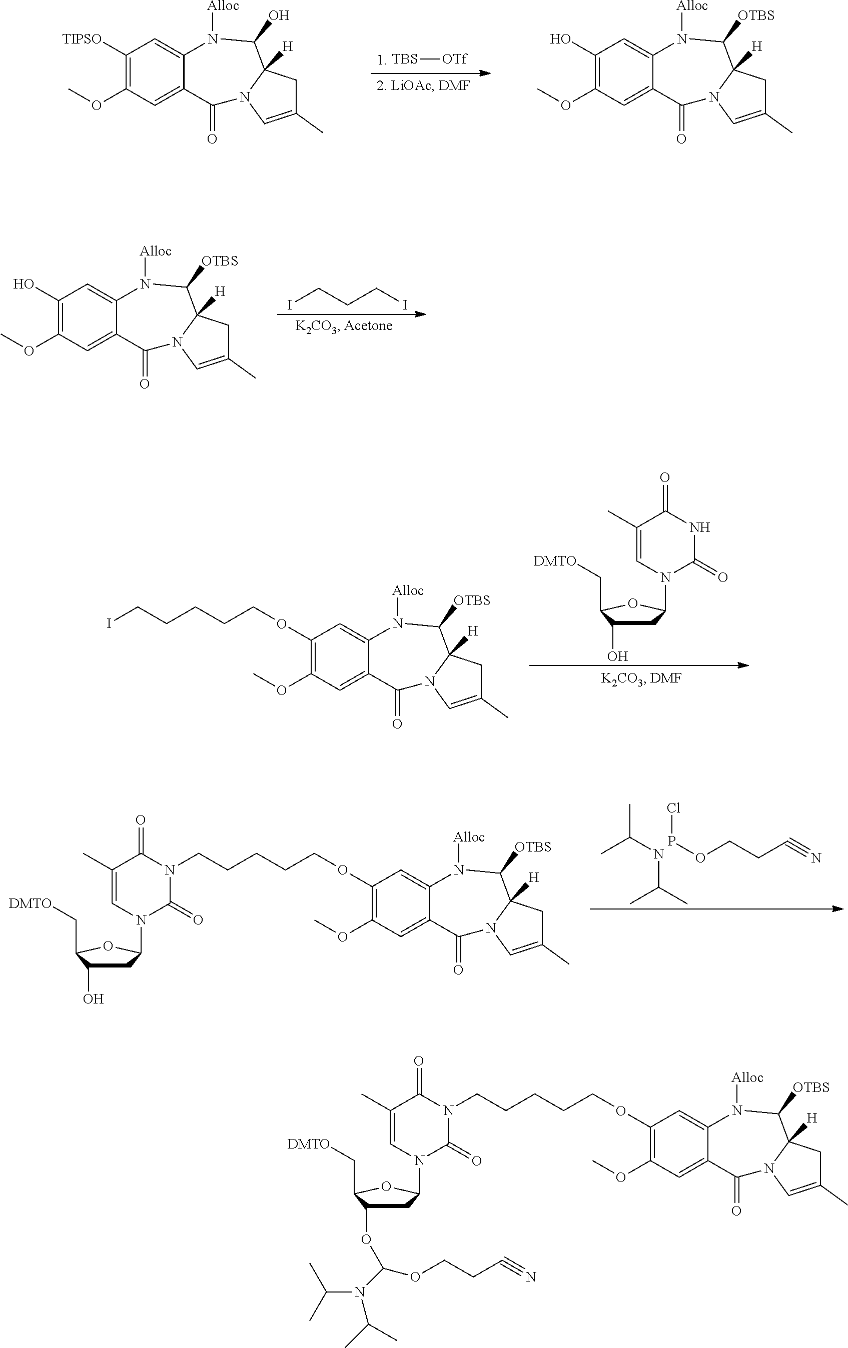

Compounds III-1-III-4 and III-6 shown in Table 5 can be synthesized similarly according to the above reaction scheme. Enantiomers or diastereomers can be synthesized in the same way except for starting with the other enantiomer or diastereomers of the starting material.

The above reaction scheme describing synthesases for a protected PBD thymidine based phospharamidite is based on a use of thymidine as a diol scaffold. Other diols such as solketal can be used to generate other protected PBD phospharamidites and the following reaction scheme may be used for the synthesis of compounds IV-1-IV-4 shown in Table 6.

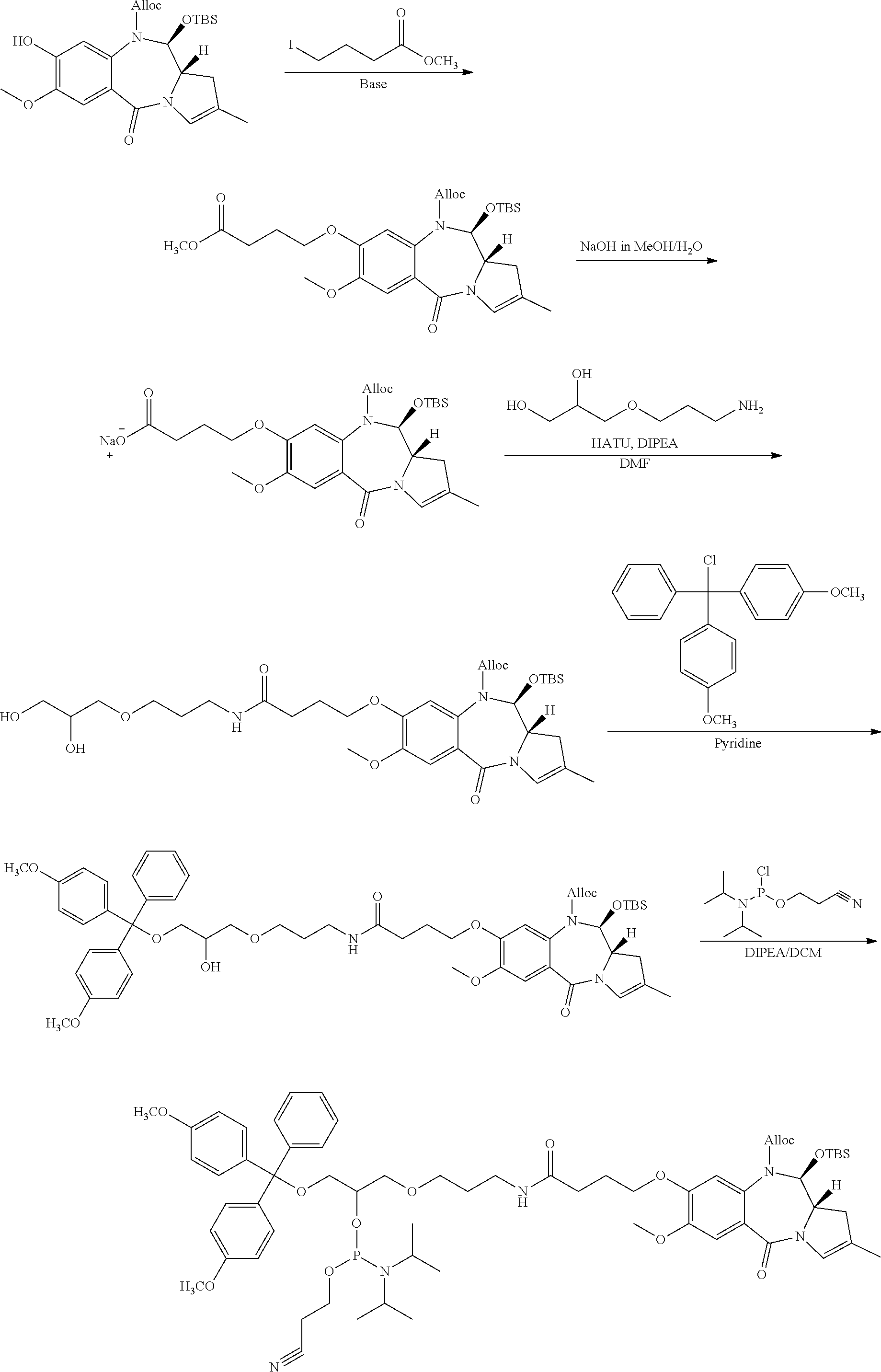

A protected PBD is added to a dried round bottom flask, under inert gas blanket, with magnetic stir bar, followed by addition of a solvent and a base. The resulting solution is stirred and then Methyl 4-iodobutyrate is added to the reaction flask. The workup affords the resulting ether product crude which is purified by a column chromatography. The ether product is treated with 0.4 M NaOH in methanol and water to afford the sodium salt.

The sodium salt (1.5 equivalent) is added to a dried round bottom flask, under inert gas blanket, with magnetic stir bar, followed by addition of DMF. The sodium salt was allowed to dissolve completely under inert gas at room temperature. Then, to the mixture is added DIPEA (3.3 equivalent), followed by addition of HATU (1.2 equivalent). 6,7-Dihydroxy-4-oxaheptylamine (1.0 equivalent) is added to a dried round bottom flask, under inert gas blanket, with magnetic stir bar, followed by addition of DMF, and dissolve completely at room temperature. Then the sodium salt reaction mixture is added to the solution containing 6,7-dihydroxy-4-oxaheptylamine; the resultant mixture is allowed to mix at under inert gas, at room temperature. At reaction completion the solvents are stripped off by rotary evaporation, under vacuum (10 mbar), with heating (55° C.). The concentrated residue is placed under full vacuum, at room temperature, for several hours resulting in crude diol.

The crude diol (1.0 equivalent) is added to a dried round bottom flask, under inert gas blanket, with magnetic stir bar, followed by addition of pyridine, anhydrous. The reaction flask is then transferred to an ice water bath (0° C.) and allowed to cool with mixing until thermally equalized (approximately 10 minutes). Then, 4,4′-Dimethoxytrityl chloride (1.5 equivalent) is added to the cooled mixture with continuous mixing under inert gas. The reaction mixture is allowed to warm to room temperature then sampled for TLC analysis. When reaction completion verified, the remaining unreacted 4,4′-Dimethoxytrityl chloride is quenched by addition of methanol to the reaction mixture (1.0 equivalent). The solvent is stripped off by rotary evaporation, under vacuum (10 mbar), with heating (55° C.). The concentrated residue is then suspended in toluene and toluene stripped off by rotary evaporation, under vacuum (10 mbar), with heating (55° C.); repeated two time. The crude produce is dissolved in dichloromethane and washed with sodium bicarbonate (saturate aq.) and separated, This process is repeated one time. The separated organic phase is washed with sodium chloride (saturated aq.) and separated. The separated organic phase is dried over sodium sulfate, anhydrous and the sodium sulfate filtered off. Solvent is removed by rotary evaporation followed by silica gel flash chromatography, resulting in a DMT protected PBD which is dried under vacuum for at least 24 hours is dissolved in dichloromethane, under inert gas blanket, with magnetic stir bar, followed by addition of DIPEA, and then addition of Cl-Phos. The reaction is allowed to mix for approximately 15 minutes and then sampled for TLC analysis (TLC showed reaction completion). When reaction completion was verified, the reaction mixture is washed by adding directly to sodium bicarbonate (saturated aq.) and organic phase separated, repeated one time. The organic phases are combined and dried over sodium sulfate, anhydrous, and then the sodium sulfate filtered off. The product containing organic phase is sampled for TLC and LC-UV/MS analysis. Then, dichloromethane is stripped off by rotary evaporation and proceeded to purification without crude weight. This crude material is then combined with crude material from a small-scale test reaction. The combined crude material is purified by silica gel solid phase extraction, dichloromethane/methanol/triethylamine mobile phase, product containing fractions were pooled (determined by TLC) sampled for TLC and LC-UV/MS analysis. The, mobile phase striped off by rotary evaporation, and then placed on vacuum line for at least 24 hours to yield DMT protected PBD phospharamidite as shown above.

The carbon chain length between the PBD moiety and phospharamidite/DMT group of the DMT protected PBD phospharamidite can be adjusted by uses of different methyl ester halides and/or diols. Enantiomers or diastereomers can be synthesized in the same way except for starting with the other enantiomer or diastereomers of the starting material.

### Examples

### General Methods

Mass spectral analysis is performed on a Waters/Micromass Quattro micro MS/MS system (in MS only mode) using MassLynx 4.1 acquisition software. Mobile phase used for LC/MS on dyes is 100 mM 1,1,1,3,3,3-hexafluoro-2-propanol (HFIP), 8.6 mM triethylamine (TEA), pH 8. Phosphoramidites and precursor molecules are also analyzed using a Waters Acquity UHPLC system with a 2.1 mm×50 mm Acquity BEH-Cis column held at 45° C., employing an acetonitrile/water mobile phase gradient. Molecular weights for monomer intermediates are obtained using tropylium cation infusion enhanced ionization on a Waters/Micromass Quattro micro MS/MS system (in MS only mode). Excitation and emission profiles experiments are recorded on a Cary Eclipse spectra photometer.

All reactions are carried out in oven dried glassware under a nitrogen atmosphere unless otherwise stated. Commercially available DNA synthesis reagents are purchased from Glen Research (Sterling, VA). Anhydrous pyridine, toluene, dichloromethane, diisopropylethyl amine, triethylamine, acetic acid, pyridine, and THE are purchased from Aldrich. All other chemicals are purchase from Aldrich or TCI and are used as is with no additional purification.

### Example 1

### Synthesis of Compound I-1

### Stock Solution Preparation

Borate buffer prepared at 250 mM, pH 10
Fluorescein-NHS solution prepared at 350 mM (300 mg in 1.35 mL DMSO:acetonitrile at 25:75)

### Solid Phase Synthesis

Compound I-1 is prepared on the DNA synthesizer via solid support using standard DNA synthesis techniques (i.e., DMT protected 2-cyanoethyl phosphoramidite). The polymer is removed from the solid support with ammonium hydroxide and lyophilized to a paste. 250 mg aliquots are reconstituted in water. A small aliquot is removed and serial dilutions are prepared in 100 mM NaCO₃ at pH 9 to determine concentration (A 263 ε=10,000). Final stock concentration is found to be 14.5 mM.

### Dye Coupling Reaction

In 50 mL centrifuge tube equipped with magnetic stir bar is placed water (1.110 μL), borate buffer (1.800 μL), Compound I-1 polymer solution (466 μL), acetonitrile (137.5 μL), triethylamine (313 μL) and fluorescein-NHS solution (675 μL). The tube is wrapped in aluminum foil and the mixture stirred overnight at room temperature.

### Size Exclusion Filtration

To an Amicon Ultra-15 Centrifugal filter (Millipore UFC900324, MW cutoff=3000) is added 1 mL of water. The crude reaction from the dye coupling reaction (4.5 mL) is added to the filtration setup. The reaction vessel is rinsed 2×with 4 mL of 100 mM NaOH and the rinseates are transferred to the filtration setup. The filtration setup is centrifuged at max speed (3220 g, swing bucket, 30 minutes). The filtrate is removed and the retentate treated with an additional 10 mL of 100 mM NaOH. The filtration setup is centrifuged as before. Again, the filtrate is removed and a third 10 mL 100 mM NaOH aliquot is added to the retentate. The setup is centrifuged as before and the filtrate removed. A fourth 10 mL 100 mM NaOH aliquot is added to the retentate and centrifuged as before. The filtrate is removed and 10 mL of water are added to the filtration setup. The mixture is centrifuged as before. The retentate is removed, the filtration vessel is washed with water and the rinseates are added to the final volume (3.5 mL). The desired product is confirmed by LC-MS and absorbance is used to determine concentration.

### Example 2

### Activation and Antibody Conjugation of Compound I-1

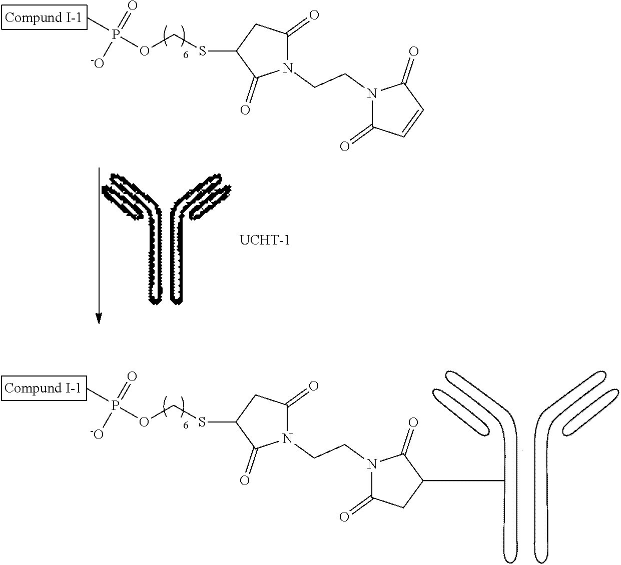

The maleimide functionalized Compound I-1 is prepared according to the method described in Example 1. In parallel, an UCHT-1 antibody is treated with bis-maleimidoethane (“BMOE”) to reduce disulfide bonds. The reduced antibody is reacted with Compound I-1 in a 5:1 molar ratio of polymer to antibody. The reaction results in a final product having a polymer to antibody ratio of 1:1 as detected by size exclusion chromatography. In some embodiments, anti-CD33, anti-CD70, or anti-CD123 may be used with bis-maleimidoethane (“BMOE”) to reduce disulfide bonds.

All of the U.S. patents, U.S. patent application publications, U.S. patent applications, foreign patents, foreign patent applications and non-patent publications referred to in this specification and/or listed in the Application Data Sheet, including U.S. Provisional Application No. 63/250,931, filed Sep. 30, 2021, are incorporated herein by reference, in their entirety to the extent not inconsistent with the present description. Aspects of the embodiments can be modified, if necessary to employ concepts of the various patents, applications and publications to provide yet further embodiments.

From the foregoing it will be appreciated that, although specific embodiments of the disclosure have been described herein for purposes of illustration, various modifications may be made without deviating from the spirit and scope of the disclosure. Accordingly, the disclosure is not limited except as by the appended claims.

What is claimed is:

1. A compound having the following structure (I): 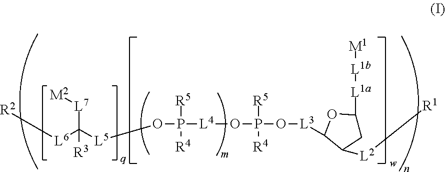
or a stereoisomer, pharmaceutically salt or tautomer thereof, wherein:
M¹ is, at each occurrence, independently absent, a moiety comprising a pyrrolobenzodiazepine, a minor groove binding agent or a fluorescent dye, provided that at least one occurrence of M¹ is a pyrrolobenzodiazepine;
M² is, at each occurrence, independently a moiety comprising a pyrrolobenzodiazepine, a minor groove binding agent or a fluorescent dye;
L^{1a} is, at each occurrence, independently a heteroarylene linker;
L^{1b} is, at each occurrence, independently H when M¹ is absent or an alkylene, alkenylene, alkynylene, heteroalkylene, heteroalkenylene or heteroalkynylene linker when M¹ is a pyrrolobenzodiazepine or a fluorescent dye;
L², L³, L⁵, L⁶ and L⁷ are, at each occurrence, independently optional alkylene, alkenylene, alkynylene, heteroalkylene, heteroalkenylene or heteroalkynylene linkers;
L⁴ is, at each occurrence, independently an alkylene, alkenylene, alkynylene, heteroalkylene, heteroalkenylene or heteroalkynylene linker;
R¹ and R² are each independently H, OH, SH, alkyl, alkoxy, alkylether, heteroalkyl, —OP(═Rₐ)(R_{b})R_{c}, Q, or a protected form thereof, L′ or a minor groove binding agent;
R³ is, at each occurrence, independently H, alkyl or alkoxy;
R⁴ is, at each occurrence, independently O⁻, S⁻, OR_{d} or SR_{d};
R⁵ is, at each occurrence, independently oxo, thioxo or absent;
Rₐ is O or S;
R_{b} is OH, SH, O⁻, S⁻, OR_{d} or SR_{d};
R_{c} is OH, SH, O⁻, S⁻, OR_{d}, OL′, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether or thiophosphoalkylether;
R_{d} is a counter ion;
Q is, at each occurrence, independently a moiety comprising a reactive group, or protected form thereof, capable of forming a covalent bond with an analyte molecule, a targeting moiety, a solid support or a complementary reactive group Q′;
L′ is, at each occurrence, independently a linker comprising a covalent bond to Q, a linker comprising a covalent bond to a targeting moiety, a linker comprising a covalent bond to an analyte molecule, a linker comprising a covalent bond to a solid support, a linker comprising a covalent bond to a solid support residue, a linker comprising a covalent bond to a nucleoside or a linker comprising a covalent bond to a further compound of structure (I);
m is, at each occurrence, an integer of zero or greater;
n is an integer of one or greater; and
q and w are, at each occurrence, independently 0 or 1, provided that at least one of q or w is 1 at one occurrence.

2. The compound of claim 1, wherein at least one occurrence of L^{1a} is an optionally substituted 5-9 membered heteroarylene linker.

3. The compound of any one of claims 1-2, wherein L^{1a} is, at each occurrence independently a pyrimidine.

4. The compound of any one of claims 1-3, wherein L^{1a} is, at each occurrence, independently cytosine or thymine.

5. The compound of any one of claims 1-4, wherein L^{1a} is, at each occurrence, independently selected from cytosine and thymine such that the compound comprises a sequence of cytosine and thymine bases capable of triplex formation with a target DNA sequence.

6. The compound of any one of claims 1-5, wherein L^{1a} has the following structure:

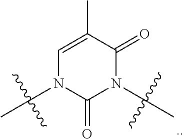 [SMILES: *n1cc(C)c(=O)n(*)c1=O]

7. The compound of any one of claims 1-6, wherein the compound has the following structure (Ia):

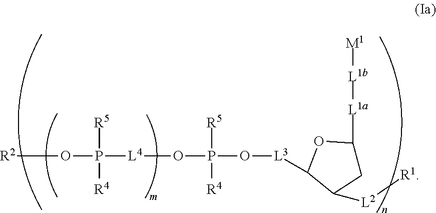

8. The compound of any one of claims 1-7, wherein at least one occurrence of L³ is an alkylene linker.

9. The compound of any one of claims 1-8, wherein L³ is an alkylene linker at each occurrence.

10. The compound of any one of claims 1-9, wherein at least one occurrence of L² is absent.

11. The compound of any one of claims 1-10, wherein L² is absent at each occurrence.

12. The compound of any one of claims 1-11, wherein at least one occurrence of L⁵ or L⁶ is heteroalkylene.

13. The compound of any one of claims 1-12, wherein at least one occurrence of L⁴ comprises alkylene oxide.

14. The compound of any one of claims 1-13, wherein at least one occurrence of L⁵ or L⁶ comprises alkylene oxide.

15. The compound of any one of claims 13-14, wherein the alkylene oxide is ethylene oxide.

16. The compound of claim 15, wherein the ethylene oxide is polyethylene oxide.

17. The compound of any one of claims 1-16, wherein at least one occurrence of R³ is H.

18. The compound of any one of claims 1-17, wherein the compound has the following structure (Ib): 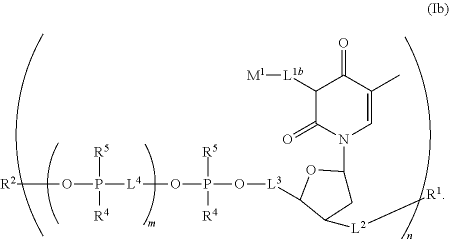
wherein:
L^{1b} is, at each occurrence, independently an optionally alkylene or an optionally heteroalkylene linker.

19. The compound of any one of claims 1-18, wherein the compound has the following structure (Ic): 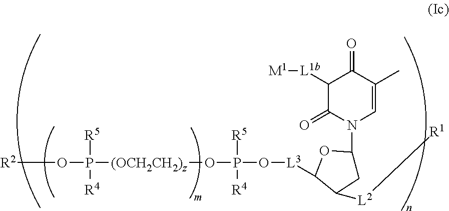
wherein:
z is an integer from 1 to 100.

20. The compound of any one of claims 1-19, wherein M¹ has one of the following structures: 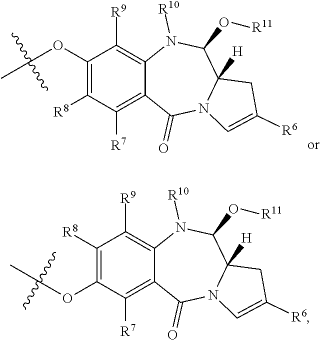
wherein:
R⁶ is, at each occurrence, independently H, CH═CHCONH2, alkyl, alkoxy, heteroalkyl, heteroalkoxy, alkylether, alkoxyalkylether, cycloalkyl, heterocyclyl, aryl, or heteroaryl;
R⁷, R⁸, and R⁹ are, at each occurrence, independently H, OH, OR_{f}, SH, SR_{f}, NH₂, NHR_{f}, NR_{f}R_{g}, alkyl, alkoxy, alkylether, or heteroalkyl;
R¹⁰ is, at each occurrence, independently a nitrogen protecting group or H;
R¹¹ is, at each occurrence, independently an oxygen protecting group, alkyl, or H; and
R_{f} and R_{g} are, at each occurrence, independently alkyl, heterocyclyl, or aryl.

21. The compound of any one of claims 1-20, wherein the compound has one of the following structures (Id) or (Ie): 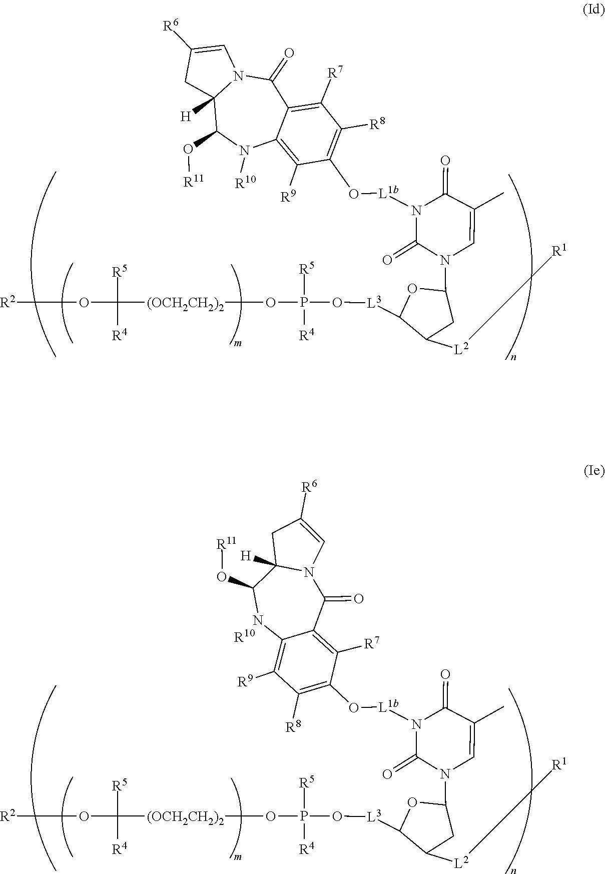
wherein:
R⁶ is, at each occurrence, independently an alkyl;
R⁷ and R⁹ are, at each occurrence, H;
R⁸ is, at each occurrence, independently OR_{f};
R¹⁰ is, at each occurrence, independently a nitrogen protecting group;
R¹¹ is, at each occurrence, independently an oxygen protecting group; and
R_{f} is an alkyl.

22. The compound of any one of claims 1-21, wherein R⁵ is, at each occurrence, independently OH, O⁻ or OR_{d}.

23. The compound of any one of claims 1-22, wherein R⁴ is, at each occurrence, oxo.

24. The compound of any one of claims 1-23, wherein R¹ and R² are each independently OH or —OP(═Rₐ)(R_{b})R_{c}.

25. The compound of any one of claims 1-24, wherein one of R¹ or R² is OH or —OP(═Rₐ)(R_{b})R_{c}, and the other of R¹ or R² is Q or a linker comprising a covalent bond to Q.

26. The compound of any one of claims 1-25, wherein R¹ and R² are each independently —OP(═Rₐ)(R_{b})R_{c}.

27. The compound of any one of claims 24-26, wherein R_{c} is OL′.

28. The compound of claim 27, wherein L′ is a heteroalkylene linker to: Q, a targeting moiety, an analyte molecule, a solid support, a solid support residue, a nucleoside or a further compound of structure (I).

29. The compound of claim 28, wherein L′ comprises an alkylene oxide or phosphodiester moiety, or combinations thereof.

30. The compound of claim 29, wherein L′ has the following structure: 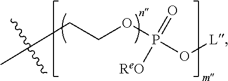
wherein:
m″ and n″ are independently an integer from 1 to 10;
R^{e} is H, an electron pair or a counter ion; and
L″ is R^{e} or a direct bond or linkage to: Q, a targeting moiety, an analyte molecule, a solid support, a solid support residue, a nucleoside or a further compound of structure (I).

31. The compound of claim 28-30, wherein the targeting moiety is an antibody, cell surface receptor antagonist, or cell surface receptor antagonist.

32. The compound of claim 31, wherein the targeting moiety is a monoclonal antibody.

33. The compound of claim 32, wherein the monoclonal antibody is Abciximab, Adalimumab, Alemtuzumab, Alirocumab, Avibactam, Basiliximab, Benralizumab, Bezlotoxumab, Blinatumomab, Brodalumab, Burosumab, Canakinumab, Caplacizumab, Certolizumab pegol, Daclizumab, Denosumab, Dupilumab, Eculizumab, Emicizumab, Erenumab, Evolocumab, Fremanezumab, Galcanezumab, Golimumab, Guselkumab, Ibalizumab, Idarucizumab, Infliximab, Itolizumab, Ixekizumab, Lanadelumab, Lokivetmab, Mepolizumab, Natalizumab, Obiltoxaximab, Ocrelizumab, Omalizumab, Palivizumab, Ranibizumab, Raxibacumab, Reslizumab, Rmab, Rovelizumab, Ruplizumab, Sarilumab, Secukinumab, Tildrakizumab, Thiomab, Tocilizumab, Ustekinumab, Vedolizumab, Abrilumab, Actoxumab, Aducanumab, Afasevikumab, Afelimomab, Anifrolumab, Anrukinzumab (IMA-638), Aselizumab, Atorolimumab, Bapineuzumab, BCD-100, Bertilimumab, Besilesomab, Biciromab, Bimagrumab, Bimekizumab, Birtamimab, Bleselumab, Blosozumab, Bococizumab, Brazikumab, Briakinumab, Brolucizumab, Carlumab, Carotuximab, Cedelizumab, Clazakizumab, Clenoliximab, Concizumab, Cosfroviximab, CR6261, Crenezumab, Crizanlizumab, Crotedumab, Depatuxizumab, mafodotin, Derlotuximab biotin, Dezamizumab, Diridavumab, Domagrozumab, Dusigitumab, Ecromeximab, Edobacomab, Efalizumab, Efungumab, Eldelumab, Elezanumab, Enokizumab, Eptinezumab, Erlizumab, Etrolizumab, Evinacumab, Exbivirumab, Fanolesomab, Faralimomab, Faricimab, Fasinumab, Felvizumab, Fezakinumab, Flanvotumab, Fletikumab, Flotetuzumab, Fontolizumab, Foravirumab, Frovocimab, Fulranumab, Gantenerumab, Gavilimomab, Gevokizumab, Gimsilumab, Gomiliximab, Gosuranemab, Ianalumab, Inclacumab, Inolimomab, Iomab-B, Keliximab, Lampalizumab, Landogrozumab, Larcaviximab, Lebrikizumab, Lenvervimab, Lerdelimumab, Letolizumab, Libivirumab, Ligelizumab, Lodelcizumab, Lulizumab pegol, Marstacimab, Mavrilimumab, Metelimumab, Mirikizumab, Motavizumab, Muromonab CD3, Nebacumab, Nemolizumab, NEOD001, Nirsevimab, Odulimomab, Olendalizumab, Olokizumab, OMS721, Opicinumab, Orticumab, Otelixizumab, Otilimab, Oxelumab, Ozanezumab, Ozoralizumab, Pagibaximab, Panobacumab, Pascolizumab, Pateclizumab, PDR001, Perakizumab, Pexelizumab, Placulumab, Plozalizumab, Ponezumab, Porgaviximab, Prasinezumab, Priliximab, PRO 140, Quilizumab, Rafivirumab, Ralpancizumab, Ranevetmab, Ravagalimab, Ravulizumab, Refanezumab, Regavirumab, Relatlimab, Rinucumab, Risankizumab, Roledumab, Romosozumab, Rontalizumab, SA237, Satralizumab, Sevirumab, SHP647, Sifalimumab, Simtuzumab, Siplizumab, Sirukumab, Solanezumab, Sonepcizumab, Spartalizumab, Stamulumab, Sulesomab, Suptavumab, Sutimlimab, Suvizumab, Suvratoxumab, Tadocizumab, Talizumab, Tamtuvetmab, Tanezumab, Tefibazumab, Telimomab aritox, Teneliximab, Teplizumab, Teprotumumab, Tezepelumab, Tibulizumab, Toralizumab, Tralokinumab, Trevogrumab, Tuvirumab, Ulocuplumab, Urtoxazumab, Varisacumab, Vepalimomab, Vesencumab, Visilizumab, Vobarilizumab, Zolimomab aritox, trastuzumab, gemtuzumab, brentuximab, vorsetuzumab, lorvotuzumab, cantuzumab, bivatuzumabor inotuzumab, or vadastuximab.

34. The compound of any one of claims 1-30, wherein R¹ or R² has one of the following structures:

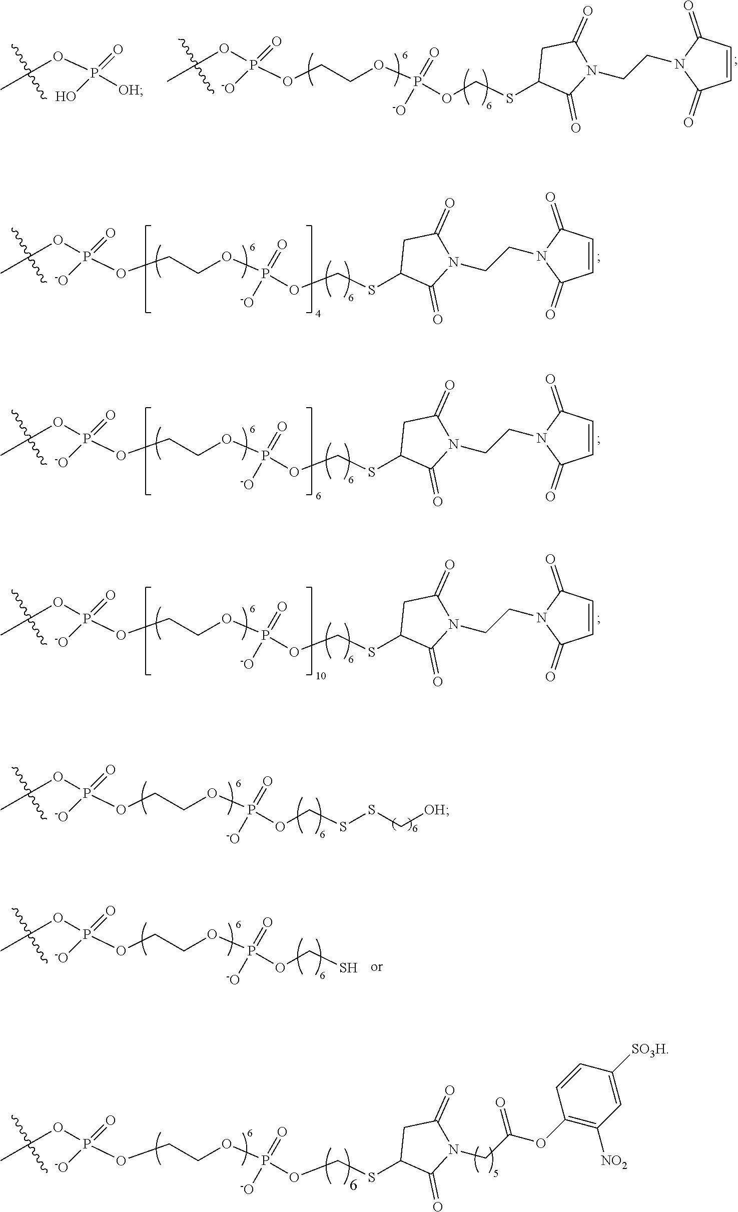

35. The compound of any one of claims 1-30, wherein R¹ or R² has the following structure:

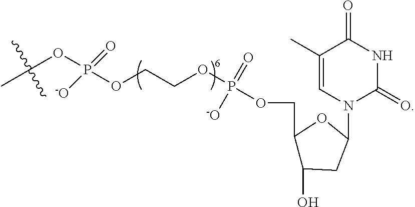

36. The compound of any one of claims 1-30, wherein M², R¹ or R² comprises a minor groove binding agent.

37. The compound of claim 36, wherein the minor groove binding agent has one of the following structures: 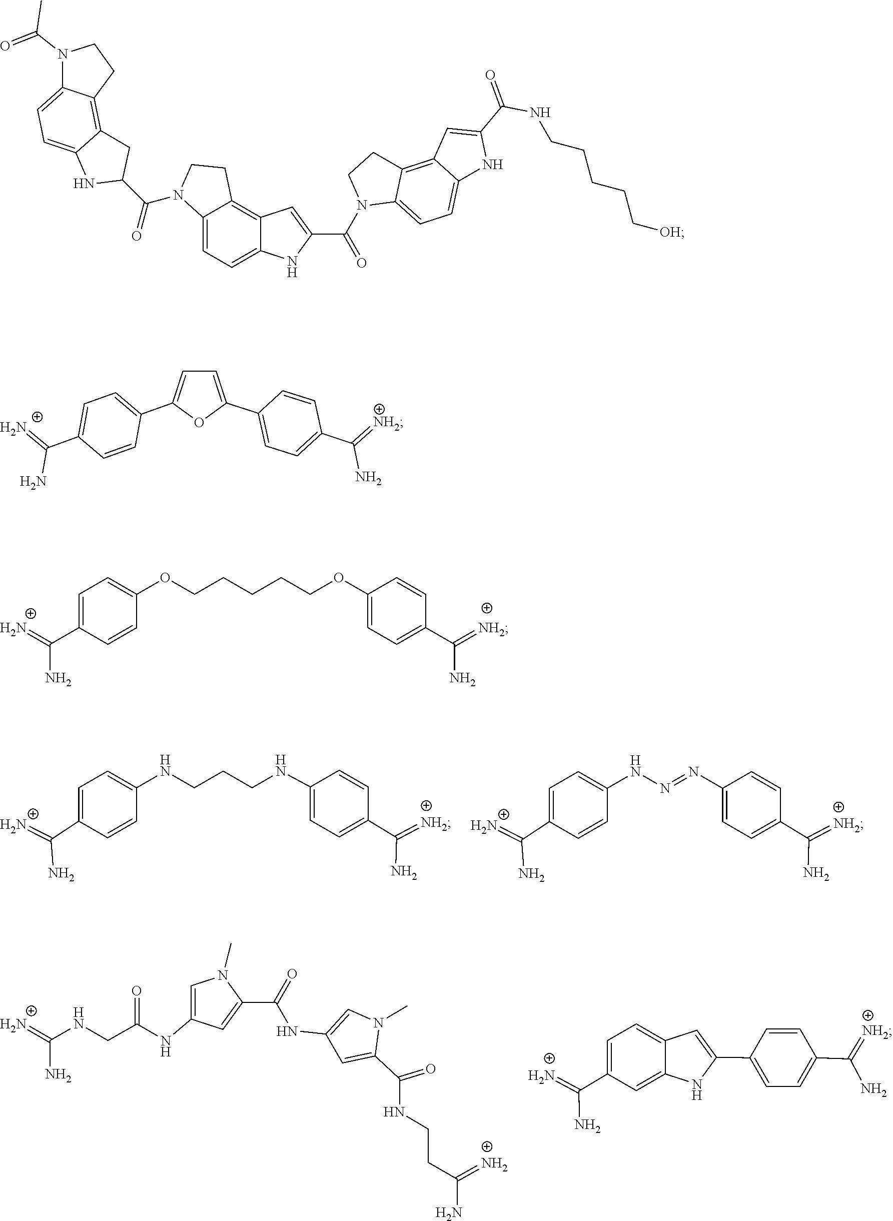 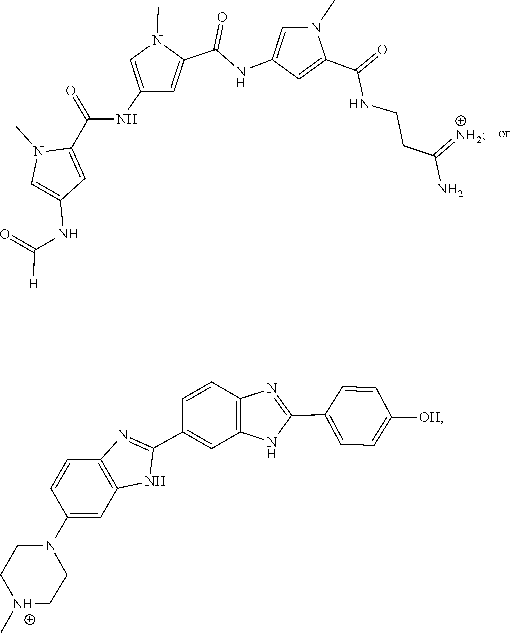
wherein one substitutable position of the minor groove binding agent is covalently bound to the remainder of the compound via an optional linker.

38. The compound of any one of claims 1-30, wherein Q comprises a nucleophilic reactive group, an electrophilic reactive group or a cycloaddition reactive group.

39. The compound of claim 38, wherein Q comprises a sulfhydryl, disulfide, activated ester, isothiocyanate, azide, alkyne, alkene, diene, dienophile, acid halide, sulfonyl halide, phosphine, α-haloamide, biotin, amino or maleimide functional group.

40. The compound of claim 39, wherein the activated ester is an N-succinimide ester, imidoester or polyflourophenyl ester.

41. The compound of claim 39, wherein the azide is an alkyl azide or acyl azide.

42. The compound of any one of claims 1-30, wherein Q has the following structures:

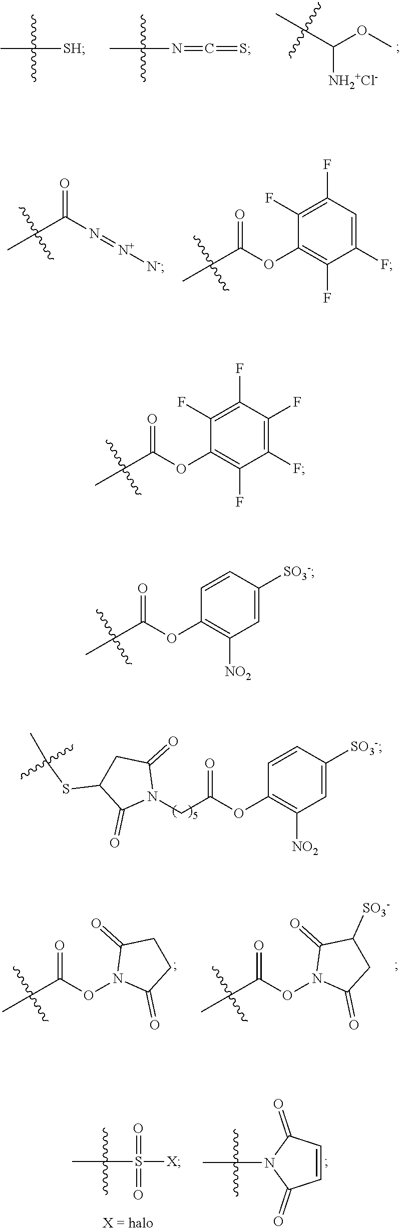

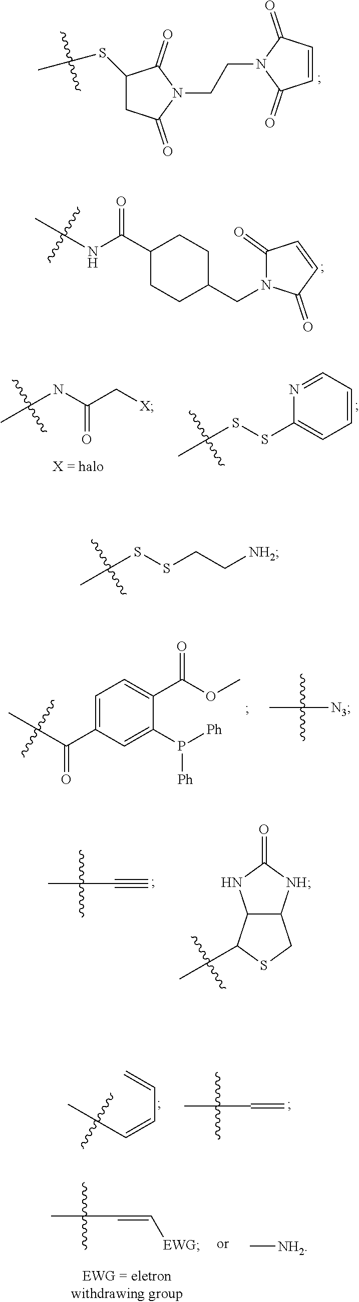

43. The compound of any one of claims 1-24, wherein one of R¹ or R² is OH or —OP(═Rₐ)(R_{b})R_{c}, and the other of R² or R³ is a linker comprising a covalent bond to an analyte molecule, a linker comprising a covalent bond to a targeting moiety or a linker comprising a covalent bond to a solid support.

44. The compound of claim 43, wherein the analyte molecule is a nucleic acid, amino acid or a polymer thereof.

45. The compound of claim 43, wherein the analyte molecule is an enzyme, receptor, receptor ligand, antibody, glycoprotein, aptamer or prion.

46. The compound of claim 43, wherein the targeting moiety is an antibody or cell surface receptor antagonist.

47. The compound of claim 43, wherein the solid support is a polymeric bead or non-polymeric bead.

48. The compound of any one of claims 1-47, wherein n is an integer from 1 to 100.

49. The compound of any one of claims 1-48, wherein n is an integer from 1 to 10.

50. The compound of any one of claims 1-49, wherein m is an integer from 7 to 12.

51. The compound of any one of claims 1-50, wherein m is an integer from 3 to 6.

52. The compound of any one of claims 1-51, wherein at least one occurrence of M¹ or M² is a nitrogen mustard, a nitrosourea, a tetrazine, an aziridine, a cisplatin or cisplatin derivative, or a non-classical alkylating agent.

53. The compound of any one of claims 1-52, wherein at least one occurrence of M¹ or M² is mechlorethamine, cyclophosphamide, melphalan, chlorambucil, ifosfamide, busulfan, N-nitroso-N-methylurea (MNU), carmustine (BCNU), lomustine (CCNU), semustine (MeCCNU), fotemustine, streptozotocin, dacarbazine, mitozolomide, temozolomide, thiotepa, mytomycin, diaziquone (AZQ), cisplatin, carboplatin, oxaliplatin, procarbazine, or hexamethylmelamine.

54. The compound of any one of claims 1-53, wherein at least one occurrence of M¹ or M² has one of the following structures:

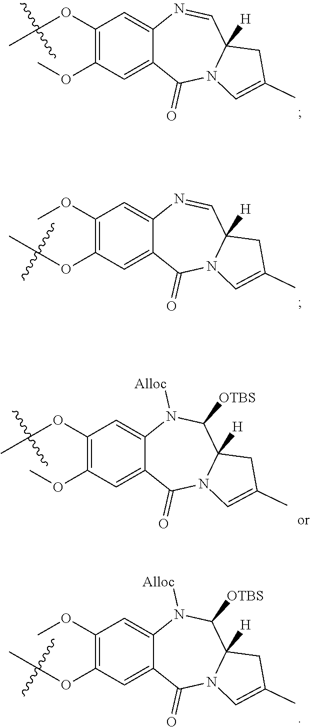

55. The compound of any one of claims 1-54, wherein the fluorescent dye of M¹ and M² are, at each occurrence, independently a moiety comprising four or more aryl or heteroaryl rings, or combinations thereof.

56. The compound of any one of claims 1-55, wherein the fluorescent dye of M¹ and M², at each occurrence, independently comprise a fused-multicyclic aryl moiety comprising at least four fused rings.

57. The compound of any one of claims 1-55, wherein the fluorescent dye of M¹ and M² are, at each occurrence, independently a dimethylaminostilbene, quinacridone, fluorophenyl-dimethyl-BODIPY, his-fluorophenyl-BODIPY, acridine, terrylene, sexiphenyl, porphyrin, benzopyrene, (fluorophenyl-dimethyl-difluorobora-diaza-indacene)phenyl, (bis-fluorophenyl-difluorobora-diaza-indacene)phenyl, quaterphenyl, bi-benzothiazole, ter-benzothiazole, bi-naphthyl, bi-anthracyl, squaraine, squarylium, 9, 10-ethynylanthracene or ter-naphthyl moiety.

58. The compound of any one of claims 1-55, wherein the fluorescent dye of M¹ and M² are, at each occurrence, independently p-terphenyl, perylene, azobenzene, phenazine, phenanthroline, acridine, thioxanthrene, chrysene, rubrene, coronene, cyanine, perylene imide, or perylene amide or derivative thereof.

59. The compound of any one of claims 1-55, wherein the fluorescent dye of M¹ and M² are, at each occurrence, independently a coumarin dye, resorufin dye, dipyrrometheneboron difluoride dye, ruthenium bipyridyl dye, energy transfer dye, thiazole orange dye, polymethine or N-aryl-1,8-naphthalimide dye.

60. The compound of any one of claims 1-55, wherein the fluorescent dye of M¹ and M² are, at each occurrence, independently pyrene, perylene, perylene monoimide or 6-FAM or derivative thereof.

61. The compound of any one of claims 1-55, wherein the fluorescent dye of M¹ and M², at each occurrence, independently has one of the following structures:

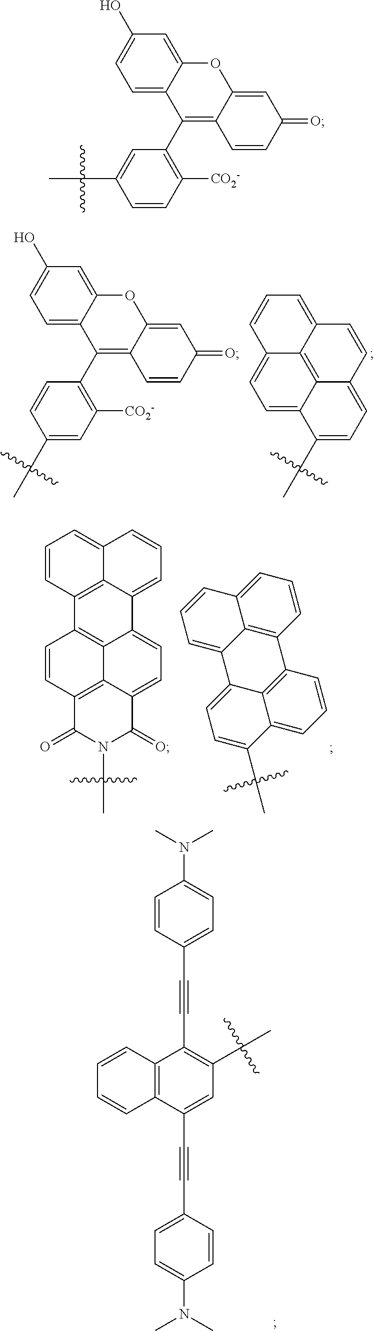

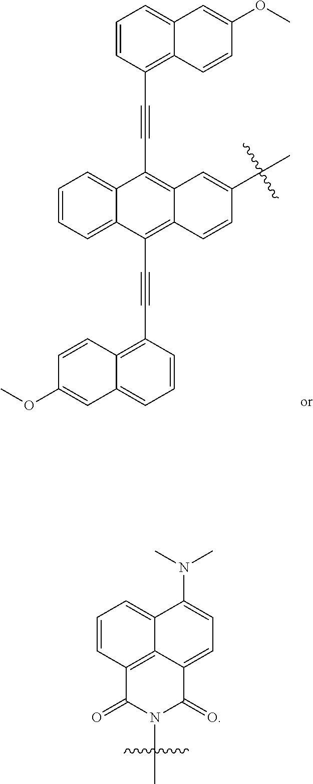

62. The compound of any one of claims 1-61, wherein at least one occurrence of L⁷ comprises one of the following structures:

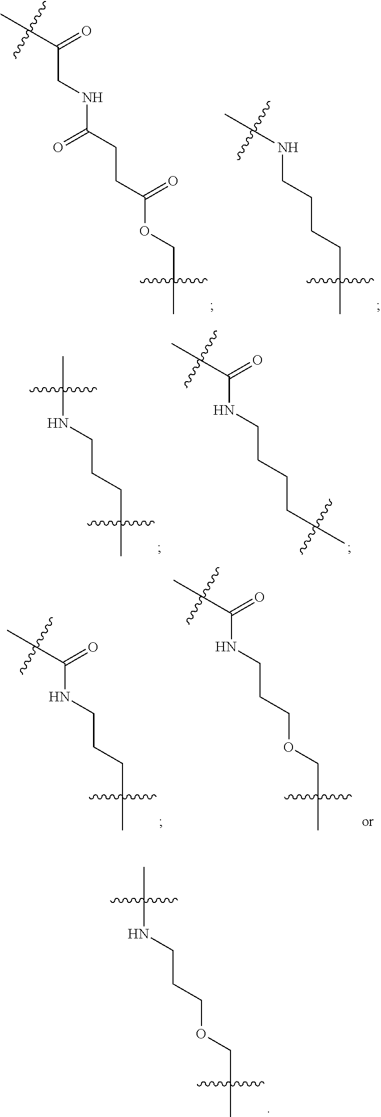

63. A pharmaceutical composition comprising the compound of any one of claims 1-62, and a pharmaceutically acceptable carrier, diluent, or excipient.

64. A method of treating a disease or disorder, comprising administering a therapeutically effective amount of a compound of any one of claims 1-62, or the pharmaceutical composition of claim 63, to a subject in need thereof.

65. The method of claim 64, wherein the disease or disorder is cancer.

66. The method of claim 65 wherein the cancer is breast cancer, stomach cancer, lung cancer, ovarian cancer, lymphoma, and bladder cancer.

67. A compound having one of the following structures (II) or (III): 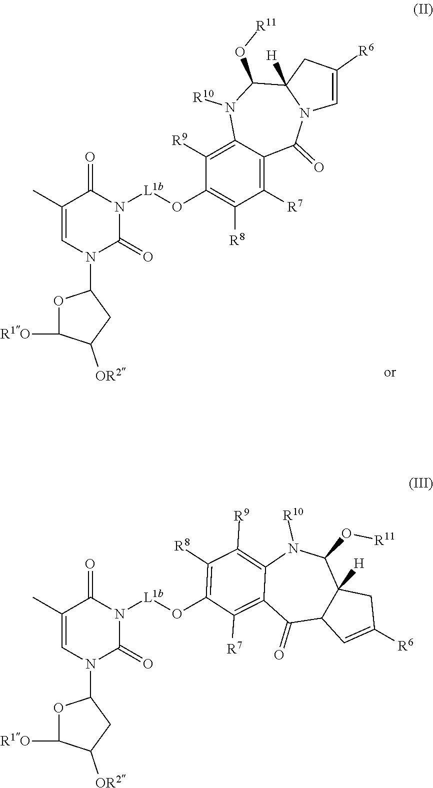
or a stereoisomer,
wherein:
R¹″ is H, a protecting group, or an activated phosphorus moiety;
R²″ is H or has the following structure: 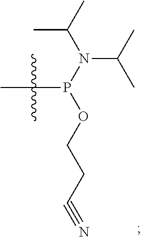 [SMILES: *P(OCCC#N)N(C(C)C)C(C)C]
R⁶ is H, CH═CHCONH2, alkyl, alkoxy, heteroalkyl, heteroalkoxy, alkylether, alkoxyalkylether, cycloalkyl, heterocyclyl, aryl, or heteroaryl;
R⁷, R⁸, and R⁹ are independently H, OH, OR_{f}, SH, SR_{f}, NH₂, NHR_{f}, NR_{f}R_{g}, alkyl, alkoxy, alkylether, or heteroalkyl;
R¹⁰ is a nitrogen protecting group or H;
R¹¹ is an oxygen protecting group, alkyl, or H;
R_{f} and R_{g} are independently alkyl, heterocyclyl, or aryl; and
L^{1b} is an optionally alkylene or an optionally heteroalkylene linker.

68. The compound of claim 67, wherein R¹″ is H or 4,4′-dimethoxytrityl.

69. The compound of any one of claims 67-68, wherein:
R⁶ is an alkyl;
R⁷ and R⁹ are H;
R⁸ is OR_{f};
R¹⁰ is a nitrogen protecting group;
R¹¹ is an oxygen protecting group; and
R_{f} is an alkyl.

70. The compound of any one of claims 67-69, wherein L^{1b} is an alkylene linker.

71. The compound of claim 70, wherein the alkylene linker of L^{1b} has odd number of carbon atoms.

72. The compound of claim 71, wherein the alkylene linker of L^{1b} is C₃ alkyl linker.

73. The compound of claim 71, wherein the alkylene linker of L^{1b} is C₅ alkyl linker.

74. The compound of claim 70, wherein the alkylene linker of L^{1b} has even number of carbon atoms.

75. The compound of claim 74, wherein the alkylene linker of L^{1b} is C₂ alkyl linker.

76. The compound of claim 74, wherein the alkylene linker of L^{1b} is C₄ alkyl linker.

77. The compound of any one of claims 67-70, wherein the compound has one of the following structures (IIa) or (IIIa): 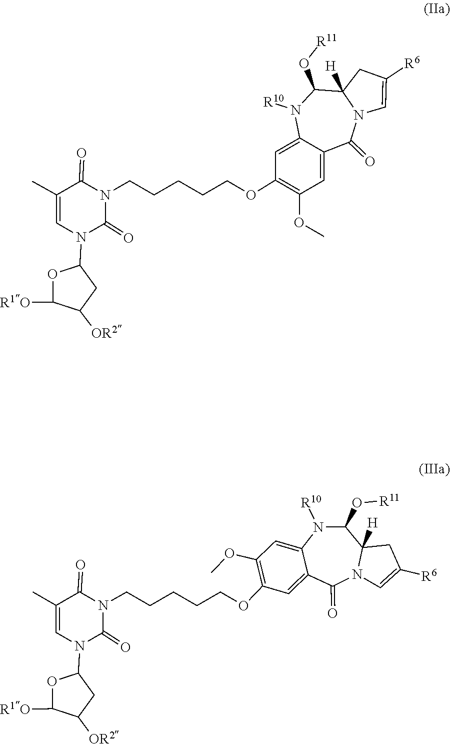
or
or a stereoisomer,
wherein:
R¹″ is H, a protecting group, or an activated phosphorus moiety;
R²″ is H or has the following structure: 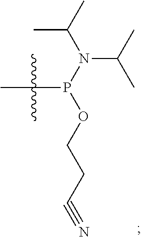 [SMILES: *P(OCCC#N)N(C(C)C)C(C)C]
R⁶ is a methyl;
R¹⁰ is a nitrogen protecting group or H; and
R¹¹ is an oxygen protecting group or H.
